SEQUENCE LISTING (17 sequences)

SEQ ID NO: 1 - DNA, 840 bp, Homo sapiens
cacacctgcaagctgcgtgcaaccgaaaggctggcgacctgtagcttgtcagattcagatatggtgcaggtgagggcctggcaaacatttagtttaatttctgagaccgttagagacaaggttaaattccaggtgaacgcggttccagagtggctgggattcccactctcagatgggaacccttcagacaagagtaaaacaggagggacttcttcgccgcccgccctgcggtgtgtgccacggaggccgctgggtgggggcgtccaaatgtggaagcgttcctccagcatgcgtggatatcctgaggactcacggacaccgattccctcttccgggcccagacgtcctaattcccgtcccccttgaggtgagatcccgttttcccccagccaaagggactccggagccgcattcacctgacttggctgccagcgcccgccttcctcgagttctcagggcaaggggctgtggtgcattttctggggatggtttgtggatgtggaagagggaaatcctttcagggattttagtcatttggcaattggtgattttcatatgacgtgctggttttaggggccaactctgataagctgggacgccctccccttgattctagaacagaaggcattttgggctaaggacgctcacaacccggtttcctgtcccgcgtggttcatggggcttgccctgggcctgggaggccggtggcgggagcctcccctacagtgaggtgggtggggggcgccacacggcgtctcagggagaggggagaggcggcttctgttccgggaagagggaacggtggaggccgccgttctcttgggcgcggcctctgctggg

SEQ ID NO: 2 - DNA, 640 bp, Homo sapiens
ccaaggctcccgggcctgctcccgagccgagccggggacgaggggcgacagcgctgcgcccacttactttttccttctccacgaaccccacaaacgctgtcctctcgatctccacgggctggccctgtctgtcgtagagggccaggacgaagtggaagaagttggatttccgcagattggaaggcggctgcttctcaaagtgagcccgggccagacccaccccgctgcggccaaagacgcagagttagatggctaaaccggacgccgacccgcgccccttgtcaccctgtacacacactcgaaccctcaaacacccacctcctcttcccccaaaacatccagtgggcgctcttcacgccccttcaggcgacatagacccagctgacaacgggacacacatgtcccctcatgtcctcgtcctctccgccaaggacgggtgcgtcctcagaactcgctgaagaggtgtgaaagttttgtttgggttttgcgcgcgagatgaaactctataagcatttactcctacctgacacgcactgcgtggggaggaagaaaagtggcggtggatgaagtgaacccaccttcgcggagtagaacccacagttatattccggcacccctcgaatctcacgtctcagctagc

SEQ ID NO: 3 - DNA, 529 bp, Homo sapiens
cctagggtggcaacagtactactgcagactcagtttcccggagggagccccctttgggcggattttaccaccctcctcctactgtaccagctttgaccgacacgtggacgtggttgcgggactcgtagtagacccagtcgaagccggcttccactgcgaggcgcgccagcaacccatacttgttgcggtcgcggtcagacgtagtgatgtccaaagcacggccttcgtagtggagtgaatcctgagcgtggtggccgtcctcgtcccagccctcagtcactcgtaggcgcactccgggccacatgttcatcacggcaatggccaaagcgttcacccgctccttacaacgctggcggggaataaaggagtcagtctccccaccaccacccttggggcaaaagggacctggataggagggttgattctttgttattccggccctactcttacccctcccagcttttgagtgtcctggagaaatgagaatctgagtcgatggtagtcaccaatctgcactctgttcccagga

SEQ ID NO: 4 - DNA, 457 bp, Homo sapiens
ctcaggtgaccggtctgggtgcggcgtggagatggcgagagctaagtgctatggccgcgaaagggtgaagggcaggggaggaaaaggccgaggggaggcgaacgctcaggttcacacatatgcaagtgggctctacagcggacttcgaagcatacactcaactccccaccatgcgccggcccgctgctctacccctgaaaagctctcccctggcccgcgatccttgctggcctaccccttggtcgttcccatccccctctccgcccccgcccaacctgctggcctcactcacctttgtccctttctcccatccccacccccccaccaccctcgctttctccgggcccctccctgtcgtttgtctcccttttatttccctccttcctttgggcccccagtctgcggccctaggacaacgccttcccaagctcggacccagccaccgtccgccttcatt

SEQ ID NO: 5 - DNA, 1241 bp, Homo sapiens
ccctgatcagtttgtatatgaaacactttttttcctgccttaaggttggaatctggtaagtttcagacaagtggttgcagggggagatgacccaggggaccgtgcgggacagggccggtgaacccgattccgcgaggcgggcgtctcgaagggactggtctgcgtgcctcaccgggttccccagcctggctccgaggaaaggccgcgcggaggcgagaaagggcccacatctcgagtctccagtctctccttgaaaagagcaccccctaatcccggaggaagggagggtgaaggaggaggggagagcccactacaggcaaggcaggaaggttaatcattttgttaaccggaaagggtgctgccccgcctggccgtcgtcaccgagcgtgtcagtcacccgctctcgatttcgccgcagaattacctgctcaaagagccaccccaagaagccctcagctccgaggtcttcccgcccgtcgtctgcgggaagtaaacggcgtcctccctccaaagaagtggatgccccttcgactctcctccgccccccggcggttcctaaatctaggaaagccagccccggcatcgctcactactcctgcacctcagctctgggatacccgctcagtacagacagaccatcaaccaaaaaagaaaagaagaaaagaaaagtggtggtgggggtgggggtgggcagggagcgtaaagtagcgagatcacttatttgactcttctgttttcttaggaacctgatcctggcccccggcagagcataagggcccgaggccagggacctagaggccggaccaagttcgcgcgaggtgttcacagcctaggcgccccgggcttggcttcgtactctgcaacaagccaagctcccttgccacgagtatatcagcaacttcagaaataaacaccagcgaaaccccttcccacattagcttgttgctgtgctactgacactgggcttagtcgatgttcacacctattacatttttccggaaagtgggaggagcagggggtagagaaagctgtgtgtattgagggaaggggagatgtgtgcaacaccttccagaaaaatggggaggtagtttagaaagtttttctctgtaacagcacagcttctgagaaggcagagagaagaaagcagacaggaaacgaatcccttcctcctctcccctttacccccttccttcactccctagaccaagctggtcacccggctgggaatcaagattgtgtgg

SEQ ID NO: 6 - DNA, 729 bp, Homo sapiens
aaaatgccaagagcgtgcgctctaagcctctctcgaacgcacagccacaccaaatccagtatttctgatcggccatggggccctaggtccttctcactcgaggctccaggacttcgaatttcaccagccgcccctcaccccacacctccccggaccagtgcggcggagcggggtcggtttccaggcgcgcgtacctggactcgcgcctctgtgaggtcgatcttcagggccagctcctcccgagtgtagatgtcggggtagtgagtctccgcgaagaccctttccagctctttgagctgggcactggtgaaagtggtgcggatgcgccgctgcttgcgcttctcgttgaggccgccgtggtccgtgaagagtttgtaaggaactagagtatgacagaggagacagaaagtgagcaaatcagccggcagctcgccggccgtggagctagaatgtgaggactccaaggtcaggtcatacggcagccgctacctacacccgcgcgagagagttgccgagagaagctgcgggaagaaaccgaggaaatttgttatctgcggaaacctgggctcaaacttcgggcttggcggagtcctttctggcacaagcgcctttgggtggattgaaacagcgcgatgtgacggacttgagatagtgctttcagcaggaaaaaaccagagaggaaaaaaatccaaaaacatcagtcggaatatcagggagccaggaaagaag

SEQ ID NO: 7 - DNA, 946 bp, Homo sapiens
ataaatttcacccgtgtccaagttcaccgcgcccccaaaaccgagtctggggcggcagggggaactcctggccaacgaatccatgcctcgccctcctgtgatgaacctggtacgcacggttttctggttaattctatcgctgaaaactggtgcggggggcgcacttctgagacggaagagcatctaggagctgaatcctccacgcgggtcgcccaggttgatctgaatttctggggaatggcttggctgcccgcccgggaccaggccgaccctccttgacggtggcgtagagggctggagcctgggtactgcgaggctcctcgcatggctgggcccgccgcgaggggttgcagagcggctcagggatcgattcaagcatcgtctctcctccctcgcccccagacagagctgggcgcggggttccccttccagatggagcgagggtctcggggtggccccggaaaaggggagcccgcggccacggctacgtattgccatctcgcgagcagagatgtcacctcctgcctttggaggaaagggagcccggtggggatgagcgcatttagcccaatgctgggaacaaagcgcactccgcgcttctgcgatttcgctccattttgaaatgtgttggcgctttggtggggccgctgcggtgggcaaggccgggggcgctgttaatggaggaacctcagggggacggtccttcgtaggaaactctatcctggctctgcgcgcgctttaaggaaatggcttccctccaggacctcgagggatgcagcttttgcgcggatgacggtggggtgctgaaccagccggtgcgcctctggaaatgtctgggcacggatcctggggccatcgacgactcctccccattcccagcaggcgggagctcttacattccgagcgagtgacccctctcaccctctggcgctcacacacctgtaac

SEQ ID NO: 8 - DNA, 630 bp, Homo sapiens
gtcaagatgccacctggccgatggaccccgggaagctgcatggatctcaaatgggccaagggccccgggcttggaagggcaggtggcgcctagacccagcgttcccgctccacaggtctccaggagttcgcccggcgtttcctctgcagcgggtgctactctagggtctgcgacctcccgctggactgcccaggtgagggggcggggcctcggggtgcaggaggccaacctgagaaaagggaccagagcaccgaggggcataagctggtggcgaggccagacgtggtcggggcccagcgagtgaaccctgctccgtcttcagttcaggatgtgacagtgactcggggcgaccaggctatgttttcttgcatcgtaaacttccagctgccaaaggaggagatcacctattcctggaagttcgcaggaggaggtgtgagtcggggcggggccggcgcgaagagtttagacgggcgagttagccttcactagaggttggggagtcagcttgcggggacgggactccgggaatctccgtctagacacaagcatcagcaagaggagggtcgcgggatttaggggagagctcgaagatcagaatgcaggaccaaggacctggggcgatggctcacg

SEQ ID NO: 9 - DNA, 930 bp, Homo sapiens
ctccgacggctaccgctggtgcgccaccaccgccaactacgaccgggacaagctcttcggcttctgcccgacccgaggtacctccaccctgtctaccaggttcagccccgccctctcatcatgtattggcccccaaaacgcggctcttccctcccatcagtttgtctttccactctcattggtcctcaggacgaccgtgactccgcccacctacaccacatttccaccactatccctgacttccaatggccccgccccagccactaaggttcggccttttctgcccagctggccgcctcttccttggtctggtgtcccaggcaccgcccacgggtctagcctcttctcaggagtgctctacagcgccccctaggccaccaagattgtttagctccctgtcgggtcggcccctgactccttattggactcatccatctggctcatccaaggccttgggtctctccagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggtaggcgtggtcccgcggctccggggctggggttcccggcagtggtggtggtggggtggccagggctgggggctcggcccggcgctcacgtctcaggctccctctccctccaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtgaggcaggggcaggga

SEQ ID NO: 10 - DNA, 2947 bp, Homo sapiens
cgatgcccagcgttttggccgctttcttctcccgggagaacttgaggagcctcactgagaagtgcgtcttggtcttggcgctggccatcccgctgcctcctgccggggcgtttttccgatggatgcggagcgtcacttgctccgagtccgacttgtcggtcttgaggccagacttgaggccccggctctccctcttggccaccacgtagacgcggcagtacatgaccaggatgatggccagaggcaggtagaaggagcccagcgctgagaagagcacgtagcccggctcctcgttgatctggcagatggtctcgtcctcgggggccggctgcctccagccgaacaggggtccaatggatatgaccagggagagtgcccagacgcagagcagagccatgagacccctcctctgggtgacgatggttgggtagcgcagcgggtagctcacgccgatgtagcggtcgatggagatgatgcagaggcccatgatggacgcggtgcagcacagcacatccactgccgcccagatgttgcagaagaccctgccgaaggcccagtagcctaggacctcgaagatggcggagaagggcagcaccgtggaggtgagcaggaggtcggccaccgccaggttgacgatgtagtagtgcgtgactgagtgcaggtgtcggtgacaggctacggagaggatcactaggatgttacccagcaccccgaaaagaatgaggccccccaagatcaccccgagcagaatggccttggaaatgttcaccggtgccggcggttgggtgcagttggagctgtcggaagcatttcccgagagaaacaccatggtcccagccggggccgggcgaggtccggctgtccagggccacctcccgggctggcgcggaggcgggagcgcgggagccgggaatcaaaaggtctcggctggagggagccctgccaggtgggtttggctgggggtgagagcgcgcgcgcgggtgggaaacaaccctggccagccctgggaaccctcagaaggccacatgaaggggcagggcattaaagacatggccaggggcgcgcggggctgccggggaccctctccacctgccgggctggcctagcccgggacccggactccctccctacccgaagctgggtgcgaagatccaggagactccttgcaacatgcaattccagaattacgagaatctgcttttccgcgctgtcttattcgtcctggctgggggaagattcagcattctgcacatgattcggaattcaaaactccagcgccagtctctccctcaaaccaaaagatcagccgtcgacgctcaaaggcagggaccgatggttgggtagcgcagcgctacaggttgggcgctgctcctggcccgcagttacctacattttgagctgccccaccgaaggctcctgctctctccagcttctaggagcacaggtcaggggacgtaggtgtggaatatgtgctgagacccaggaggctgcggggcatcgtttgaccgcgtccacctgaaagagcgcaaagagaaaggcggctttgagctaggcgccccagggaaagaggctgtgctgagcttgacgggttgggggacaccagttgggagccgggttggttctgcggtccagaagctgcttcgcccggcagcggtggaggcgacttcggagctcatctcgcgcccccaccactgggaacctgcctagcgcactctactgagtcacctctgcccgagttcaggatccgaagcgaaaaacaaacaaaaaaacaaatccccaaaaccccaggctccagcgctcgaagtctggatttcgagcgcaggtaccacgaaattaaaatcctcgaagccccggaggggcgactgcggctggcgagtgtgtcgcacggatcctagccggggaattctggaggatgtactcggtttccgttagaacaggttgcctcgcagtcacctggagggggcggcctgggagagggaacagctgcgacccagggacctcagggccaggaccacgagcacgctcactctcacgccggtggaattcgcactcgggtgtgcagagcgcaccggtctgtccaccagccaagctggcttgagagccaggtcccgagcgaagccgggagggacccgaagacagaaagcgacccaggtctgtccacgacgcctttccaagcctcactgttggccacccgaggggctccagctgccagccgtagcgggactggcgggggacggggtggggaggacgtccctctgcagagggagctttgcaagtgacagtcactgcggattttgcaggcgtaaacattaagccggcatgccagcgggcaggggccgccttcgattttctgggctgggggccaccatctgcgtaagaaacctgggtttccacaactgcgaacctaccgcagggactcggcaggacagcgcggcgtgaggaagtgcccaccttgccttctggggttgggccctgaggtgggacctcgggcaaagactcttgttaaaatcgcaagttggagacctcacaggtggtattaaaaacgtgccactgcagaaacccttttcctcctacctcgagggtgataaacacagggttaaaaagacacttgttcagtagaaggcaacttcgcagaagatgtaagggaatcgggggtggggtagaggggccggtataaaacctggtggaaaaagcgggaggcgctgggaaaagtgggggttccgtctcaccagacggcgggggaggtctgccctcacccactcggccctgcgggacgccggccccggcgcactcacctgaagcgccgctgctgagccaccagctcgcgcgcgggggatgtggacccggcttcggtcccgggagctgcctgcctgctcttctctggaggcggagaggggaccgtgtctccgaggcaccaaatccttgtccttttgcacccgctgactca

SEQ ID NO: 11 - DNA, 660 bp, Homo sapiens
ttgactacgaagccaggaggtaggcactgtttattttctgtttacactgagtctgcagcgcagagaggtgctcacaccacacagctggtgagggtctgacgtgagacggggacgtgtggtggtgcgccccgtcttcttcctgaggagctgagggaggtggggtggtgggaagggaggggtgtgcggagctgggggagggagcctggggcgctcacgtgcggggagccctctggcggggagggggcattggtgagggacggtgtggcctccccccgctccctgcagccctccaccctctggcgagggcagagcagcgcatcgatccgatcagtgctgggagctggagctccagatggtgggtggcctgcacggacgtgtggacaaagggaggctgtagcatgaaggcggccagcgaggcacgcgcgatgccctttgtcaccagggaaggccgccaggagcgcggagtgtgggaaccgccggctctcgtctttaggggtgctctgtgggggtggcgcaccaccatgaggccaagaggaaatcctcggccctttccacgccctggcccctctgctaggccggtggtggcctccctgtggcctcagttccttgcacccctcccctcctaaatgaagaactggaccaaagacctgggtccagaaa

SEQ ID NO: 12 - DNA, 655 bp, Homo sapiens
tatcaaacgctctgacaagtgtttacagctgctttctcgattactgaagtggctctgaaaagagcctttggggttaggtggttgatctattgcgtcccttgcacactcttacttcgagctggtgtacttggtgaccgccttggtgccctcggacacggcgtgcttggccagctcgccgggcagcagcaggcgcacggccgtctggatctcgcgggacgtgatggtggagcgcttgttgtagtgcgccaggcgggacgcctctcccgcgatgcgctcgaagatgtcgttgacgaaggagttcatgatgcccatggtcttggacgagatgccggtgtcggggtggacctgcttcagcacctcctacacgtaaacggagtagctctccttgcggctgcgcttgcgcttctcgccgtccttcttctgcactttcgtaacagcctttttggagcccttcttgggagcaggagcggatttcgctggatccggcatttttgcgcgaaaaaagagaacagagacttaaagaagtaatccgaactaccgcaaaacgggctggttatgcgctacttatagagcctgtatgcaaatgaagactcgcaaagtgctgcgctttaattggttaacttacaacggtgtcgtcagagggggtggagtctat

SEQ ID NO: 13 - DNA, 1669 bp, Homo sapiens
caccaagggagtgaacatgaaggaaggagaggatggagagctgagccctggagccctgcagtgtcagaaggttagggagtcaaaagcaattaggaaacgggcccgagaagagtgggccaagaagcagggaaggcaaaggggcaccagggtgctaggtggcggaatgggctgaaaagtacaagcagaggtgggacatagaggatgaggcagccatgtgggggcaggggagatggatggtgctgggtgggtagaggagagggaggaggagagcgggagagagaacaggctgatggctgggagttgggtgagtccatcgaagcaagcaaggaaggaaaatggaaggacattctgatgatgaattgagggctaggtggccaggcagttcttctatgactgtgaatattgatggatgacagatgggtgaatgtgtgaaggaaaaagagcaggggtgggcagatgtgggtacagagaggaactggctctggctatagagatggatggaacaggtcactggaggaacaggggagaggaaacacaaggaagggatgacatataagtggatgaaagggcaatgttatggtttgactgttcatgtcccccaaattcctgtatggaaatcctaacccctaaagtgatggtaggaggtggggctcttgagaggattaggtcatgcgtgggattggcacccttataaaaggggccccagggaggccctcggccctactggcaaatgaggacacagaagttgacagcctgcaacccagaagagggtcctgatcagaacctgactatgctggcaccctgatctcagacttcgagcctccggaacagtgagaagtaaatacccagtgtttataaaccaactaatctatgataattcattagagcaggctggacaggctcatgccactcaacttggaggcaggtgaatgggatacctgaataaagccaaggaaggaaagatgggatggatgtgcctggaaggtcagatggtcaacgagatgagtggtggggtgggtggacacatgcctgagaggaaacaagagagaatggacattttacagctgaatgattagctatattagtaaagcacgtaggcggatggtagaaagaatgaaggggatagaaggggagaatgcatggccgaaaagtggggcaaaagaaaggaaaagaaaaaaatgagagggaaaagaagaacgtgagaaatttatgcatgagcggttagagacaaggtggatgaggaaggagggaatagagaaagtgacagtggctggattgagctgacagttggggtgagctgaggcacgctgagtatgaaggaggcactcagtgaatgggtgagtgtgactcagctaggggagcagcaggaggaagggcgggtggatgtcttgaagggtgaatagatgtgttgccacatgaggtcacaggatggagggatgattgggtctattgacaggtagggcttggaaggaaagtgggcaattactgagaaagtgagactagatgaatagtttagtgggttgatgcataaggttggtggggacaggatgggagagcagggcagggaggggagcctgaatgatcatgtgtgaacacaggtggatggaaggttgggtggctatttggaggggttgaaggaaatgtgggaggatggattgaatataagttt

SEQ ID NO: 14 - DNA, 655 bp, Homo sapiens
ggaatattcttgtttgccctcctgggaccgggctgctgagcaaggtgggtgggcgagggactctgagctgaagggcagggtgaaagaccagaggggaggggagggaagggattccacctccttccctcctgggtgaggagcgggcagcgtggataattccctcggaggcgccccaacccaaatctacatacataaagcccaggctggcgttttgagtgatcggtttattaatacagcggggccgctgccgaggcggatcgcaataacaaagaaggcctctccgccacccaccagaggcgtcgttatgtaattgctccgaggaggagcagagggctggggcgcgccgcgggtgctgggggctcggccagcagggaagctgcgatccgggtctctggggatccgagccggcggcggatggagggaacacttgctggagagtcatcagcccttcacctggtgctctcctcctcccacgcaccggtcagcggtcaaggggcgggttaacaccggtttggcagggcgcagacctccgcccacccgggagaattgaccccggaggcagatctgcagacccagaggaccccagggctgacccagggcctcccctccgccactcaccaccgcccatccttgccccactctcaccctgggatgg

SEQ ID NO: 15 - DNA, 735 bp, Homo sapiens
caggagagaagcagccaggctggtggggtgggcagtagaggggactgtggggaggagggcagggcccaggggagagaggtgtcaagacgtagggcaggagtgagtctgagggtgtgggggcttcagaagtgccacggcccggggccagggccaagcgcgtgagcctttctcctctggccgctgacctgcccgcctgttccgcagtacgtggtcatccccacgcggcgcgccactgccgtggccttgcagagcttcacctcccacctgctgggggacgccgggagcccctacctcattggctttgtgagtagccccggggtggggctggccagggcaggctgggggactgcaggagggctctgccctgacatccacccccaaatcctcgcagatctcagacctgatccgccagagcactaaggactccccgctctgggagttcctgagcctgggctacgcgctcatgctctgccctttcgtcgtggtcctgggcggcatgttcttcctcgccactgcgctcttcttcgtcagcgaccgcgccagggctgagcagcagtgagtgggggggaggggaggccctgctgcaccgccgggaagcagggaccgtgatagccaccgtgagccctgccctggggtggggcggggagggtacagacctcccatgcactcagtgcacccactggttgctggggtccagccctgaggcccagtgccagggtcagcctggagctgtgg

SEQ ID NO: 16 - DNA, 640 bp, Homo sapiens
cccaaggaagagtgcccggggctggtcccgcaggtgccccggggaggcaccggcctgaggtgtgggcaccctcgcccccaggttctactgggacttcaccatgctgctgttcatggtgggaaacctcatcatcatcccagtgggcatcaccttcttcaaggatgagaccactgccccgtggatcgtgttcaacgtggtctcggacaccttcttcctcatggacctggtgttgaacttccgcaccggcattgtgatcgaggacaacacggagatcatcctggaccccgagaagatcaagaagaagtatctgcgcacgtggttcgtggtggacttcgtgtcctccatccccgtggactacatcttccttatcgtggagaagggcattgactccgaggtctacaagacggcacgcgccctgcgcatcgtgcgcttcaccaagatcctcagcctcctgcggctgctgcgcctctcacgcctgatccgctacatccatcagtgggaggaggtgaggtggggcgggggcggggccaaggcagcaggggcggggctataatggtgcatgggcggggccaaggcagcaggggcggggctataatggtgcatgggtggggccaaggcagcaggggtggggctatgagag

SEQ ID NO: 17 - DNA, 640 bp, Homo sapiens
agagagggtgggggcttgaggaactcagcgagcaggtagagtctggggagcctgggggcgaaattcagagagggagggcggtgctgggggcgggaactcagagggggagggcggggctgggggcaggactgagtgggagggtgggggcctgggaggggcgctctggggcagccggatcgttctccctccgcagccagtggtggtgattactggcgaatcttgaacccgggtgagtaccgcgtgacagcccacgcggagggctacaccccgagcgccaagacctgcaatgttgactatgacatcggggccactcagtgcaacttcatcctggctcgctccaactggaagcgcatccgggagatcatggccatgaacgggaaccggcctatcccacacatagacccatcgcgccctatgaccccccaacagcgacgcctgcagcagcgacgcctacaacaccgcctgcggcttcgggcacagatgcggctgcggcgcctcaacgccaccaccaccctaggcccccacactgtgcctcccacgctgccccctgcccctgccaccaccctgagcactaccatagagccctggggcctcataccgccaaccaccgctggctgggaggagtcggagactgagacct

(19) United States
(12) Patent Application Publication
(10) Pub. No.: US 2011/0301050 A1
(43) Pub. Date: Dec. 8, 2011

(54) DNA HYPERMETHYLATION BRAIN CANCER MARKERS

(76) Inventors: GERD P. PFEIFER, Bradbury, CA (US); XIWEI WU, Diamond Bar, CA (US); TIBOR A. RAUCH, Chicago, IL (US)

(21) Appl. No.: 13/069,128

(22) Filed: Mar. 22, 2011

Related U.S. Application Data
(60) Provisional application No. 61/316,324, filed on Mar. 22, 2010.

(51) Int. Cl. C40B 30/04; C07H 21/04; C12Q 1/68

(52) U.S. Cl. 506/9; 435/6.11; 536/23.5

(57) ABSTRACT

One aspect of the present disclosure relates to a composition comprising a DNA hypermethylation brain cancer marker on acellular DNA of a human subject. DNA hypermethylation brain cancer markers (or DNA hypermethylation markers) are frequently methylated in the DNA of brain cells and acellular DNA of individuals suffering from brain cancer. These DNA hypermethylation markers are not frequently methylated in the DNA of brain cells and acellular DNA of individuals who do not suffer from brain cancer. Another aspect of the present disclosure relates to a method of diagnosing brain cancer in a human subject comprising providing a sample containing acellular DNA from the human subject, determining whether one or more DNA hypermethylation markers on the acellular DNA are hypermethylated, and diagnosing brain cancer when the DNA hypermethylation markers are hypermethylated.

### PRIORITY CLAIM

The present application claims priority to U.S. Provisional Patent Application No. 61/316,324, filed Mar. 22, 2010, which is incorporated herein by reference.

### STATEMENT REGARDING FEDERALLY SPONSORED RESEARCH OR DEVELOPMENT

The invention was made with Government support under Grant No. CA084469 awarded by the National Institutes of Health (NIH). The Government has certain rights in the invention.

### BACKGROUND OF THE INVENTION

Human solid tumors are characterized by a broad spectrum of genetic and epigenetic alterations. Astrocytomas are the most common type of human gliomas, and are primary malignancies in the central nervous system. These tumors are classified by varying degrees of malignancy with grade IV astrocytomas (glioblastomas) being the most aggressive. Animal models and recent progress in stem cell research have now established that dividing cells in the subventricular zone (SVZ) give rise to astrocytomas (1). Human cancer genome sequencing studies have catalogued characteristic mutations in glioblastomas. These studies have uncovered that individual tumors generally have ˜30-40 mutations in gene coding sequences that alter the amino acid composition of a protein (2). The most commonly mutated or homozygously deleted genes include CDKN2, TP53, EGFR, PTEN, NF1, PIK3CA, and IDH1. At the epigenetic level, tumor specimens are most often characterized in terms of altered DNA methylation (3). Aberrations in DNA methylation patterns may have critical effects on tumor initiation and progression (4). Both DNA hypermethylation of specific genes and DNA hypomethylation commonly affecting repetitive DNA are observed in brain cancers (5-11). CpG island hypermethylation can affect a variety of different genes, ranging from regulators of cell cycle progression and apoptosis to DNA repair genes and developmental transcription factors. With regard to the latter class of genes, it has recently been recognized that a large fraction of the genes commonly methylated in human tumors represent targets of the Polycomb complex in human embryonic stem cells (12-17). In the present disclosure, we have employed a comprehensive methylation profiling technique, developed in our laboratory, and termed “methylated-CpG island recovery assay” or MIRA (12). This assay was used in conjunction with CpG island and promoter microarrays (MIRA-chip) to characterize the CpG island methylome in normal human brain tissue and brain tumors (astrocytomas) representing different stages of tumor progression and malignancy.

### BRIEF DESCRIPTION OF THE SEVERAL VIEWS OF THE DRAWINGS AND TABLES

FIG. 1: Localization of methylated CpG islands in normal human brain relative to known genes and correlation between gene methylation and gene expression levels.

A. Localization of the methylation peaks in normal brain relative to gene position. Each normal brain sample had between 6,000 and 7,000 methylation peaks. B. Correlation between 5′ gene end methylation in normal brain and gene expression levels. Public microarray gene expression data of whole brain tissue were obtained from the GNF SymAltas database. Genes with methylation peaks at their 5′ end are generally expressed at lower level than genes without methylation peaks at the 5′ end (Kolmogorov-Smirnov Test P value=1.8e-10). C. Correlation between gene body methylation in normal brain and gene expression levels. Genes with intragenic methylation peaks are generally expressed at a higher level than genes without intragenic methylation (Kolmogorov-Smirnov Test P value <2.2e-16).

FIG. 2: Confirmation of tumor-associated DNA methylation by combined bisulfite restriction (COBRA) analysis and sodium bisulfite sequencing. The CpG island associated with the BCL2L11 gene was analyzed in normal brain DNA (N) and in brain tumors (numbered). A. COBRA assays. In the COBRA assay, cleavage by BstUI indicates methylation at 5′CGCG sequences. B. Bisulfite sequencing. In the bisulfite sequencing panels, open circles represent unmethylated CpG sites and closed circles represent methylated CpG sites. The triangles indicate BstUI sites.

FIG. 3: Tumor-specifically methylated genes and tumor grade. A. The number of methylated regions is plotted according to tumor grade. Tumor-specific methylation peaks were identified. These peaks are not found in any of the six normal samples. The grey horizontal lines represent the median number in each grade. B. Cluster analysis. Methylation peaks commonly found in tumor samples but not in normal samples were identified (see detailed description). Then the data were subjected to hierarchical clustering with Euclidean distance and average linkage method using Cluster v3.0 and visualized in Java tree-view. Light grey color represents a methylated state, and black represents an unmethylated state.

FIG. 4: Clusters of hypermethylated CpG islands at the HOXD locus. The top of the Figure indicates the location of the individual HOXD genes. The associated CpG islands are shown as dark boxes. The bottom part of the Figure shows the NimbleGen array data (methylated fraction versus input) for three normal brain tissues (N) and three grade III astrocytomas (T). The methylation signal is shown plotted along the chromosome as a P value score. Therefore, the minimum number on the y-axis is 0 (when P=1). The P value score was obtained by NimbleScan software and is derived from the Kolmogorov-Smirnov test comparing the log 2 ratios (MIRA versus input) within a 750 bp window centered at each probe and the rest of the data on the array.

FIG. 5: Clusters of hypermethylated CpG islands at the histone cluster 1 and HLA loci. The top of the Figure indicates the location of the histone cluster 1 and MHC genes. The location of Refseq genes and mRNAs is according to the UCSC Genome Browser. The associated CpG islands are shown as vertical bars. The bottom part of the Figure shows the NimbleGen array data (methylated fraction versus input) for three normal brain tissues (N) and three grade III astrocytomas (T). At this level of resolution, each bar represents a methylated CpG island. The methylation signal is shown plotted along the chromosome as a P value score.

FIG. 6: Confirmation of tumor-associated DNA methylation by combined bisulfite restriction (COBRA) analysis and sodium bisulfite sequencing. The CpG island associated with the ZNF274 gene was analyzed in normal brain DNA (N) and in brain tumors (numbered). A. COBRA assays. In the COBRA assay, cleavage by BstUI indicates methylation at 5′CGCG sequences. B. Bisulfite sequencing. In the bisulfite sequencing panels, open circles represent unmethylated CpG sites and closed circles represent methylated CpG sites. The triangles indicate BstUI sites.

FIG. 7: Confirmation of tumor-associated DNA methylation by combined bisulfite restriction (COBRA) analysis and sodium bisulfite sequencing. The CpG island associated with the RAX gene was analyzed in normal brain DNA (N) and in brain tumors (numbered). A. COBRA assays. In the COBRA assay, cleavage by BstUI indicates methylation at 5′CGCG sequences. B. Bisulfite sequencing. In the bisulfite sequencing panels, open circles represent unmethylated CpG sites and closed circles represent methylated CpG sites. The triangles indicate BstUI sites.

FIG. 8: Overlap of the most commonly methylated genes and gene marking by the Polycomb complex. Methylation data is for the 288 commonly methylated genes, i.e. those methylated in >25% of the tumors. Data for Polycomb marking as indicated by occupancy with H3K27me3 in embryonic stem cells was obtained from Lee et al., Cell 2006; 125:301-313. The overlap is statistically highly significant (P<2.2e-16).

FIG. 9: Clusters of hypermethylated CpG islands at the HOXC locus. A view of the UCSC Genome Browser is shown at the top indicating the location of the individual HOXC genes. The associated CpG islands are shown as vertical bars. The bottom part of the Figure shows the NimbleGen array data (methylated fraction versus input) for three normal brain tissues (N) and three grade III astrocytomas (T). The methylation signal is shown plotted along the chromosome as a P value score.

FIG. 10: Clusters of hypermethylated CpG islands at the BARHL2 locus. A view of the UCSC Genome Browser is shown at the top indicating the location of the gene. The associated CpG islands are shown as boxes. The bottom part of the Figure shows the NimbleGen array data (methylated fraction versus input) for three normal brain tissues (N), three grade III astrocytomas (top three T scans) and three glioblastomas (bottom three T scans). The methylation signal is shown plotted along the chromosome as a P value score.

FIG. 11: Clusters of hypermethylated CpG islands at the PITX2 locus. A view of the UCSC Genome Browser is shown at the top indicating the location of the gene. The associated CpG islands are shown as boxes. The bottom part of the Figure shows the NimbleGen array data (methylated fraction versus input) for three normal brain tissues (N), three grade III astrocytomas (top three T scans) and two glioblastomas (bottom two T scans). The methylation signal is shown plotted along the chromosome as a P value score.

FIG. 12: Clusters of hypermethylated CpG islands at the SIM1 locus. A view of the UCSC Genome Browser is shown at the top indicating the location of the gene. The associated CpG islands are shown as boxes. The bottom part of the Figure shows the NimbleGen array data (methylated fraction versus input) for three normal brain tissues (N) and three grade III astrocytomas (T). The methylation signal is shown plotted along the chromosome as a P value score.

FIG. 13: Clusters of hypermethylated CpG islands on chromosome 14 including several homeobox genes. A diagram at the top indicates the location of the genes with chromosomal coordinates. A close-up view is shown in the lower part of the Figure. The bottom parts of the Figure panels show the NimbleGen array data (methylated fraction versus input) for three normal brain tissues (N) and three grade III astrocytomas (T). The methylation signal is shown plotted along the chromosome as a value score.

FIG. 14: Clusters of hypermethylated CpG islands on chromosome 1 spanning an area of segmental duplications. A view of the UCSC Genome Browser is shown at the top indicating the location of the individual genes. CpG islands are shown as vertical bars. Segmental duplications with >90% similarity to other chromosomal segments are shown as striped grey or black bars. Information on gene order and duplications was taken from the UCSC Genome Browser, March 2006 assembly (http://genome.ucsc.edu/cgi-bin/hgGateway). The bottom part of the Figure shows the NimbleGen array data (methylated fraction versus input) for three normal brain tissues (N) three grade III astrocytomas (top three T scans) and three glioblastomas (bottom three T scans). At this level of resolution, each bar represents a methylated CpG island. The methylation signal is shown plotted along the chromosome as a P value score.

FIG. 15: Model for astrocytoma initiation in neural stem cells. The contribution from DNA hypermethylation events is the permanent silencing of neuronal differentiation genes thus locking the stem cells and their derivative transitamplifying cells into a prolonged undifferentiated state and allowing additional oncogenic events to occur (either genetic or epigenetic) leading to initiation of malignant transformation and progression towards astrocytoma.

Table 1: Frequently methylated regions in brain

Table 2: Frequently methylated regions in grade I tumors.

Table 3: Functional annotation of 5′-end methylated genes in tumor samples.

Table 4: Genes methylated at the 5′ end in normal brain.

Table 5: Frequently methylated 5′ gene ends in brain tumors.

Table 6: Genes with frequent hypomethylation at the 5′ end.

### DETAILED DESCRIPTION OF THE INVENTION

One aspect of the present disclosure relates to a composition of matter comprising a DNA hypermethylation brain cancer marker on acellular DNA of a human subject. DNA hypermethylation brain cancer markers (or DNA hypermethylation markers) are frequently methylated in brain cancer and not frequently methylated in normal brain. Some examples of DNA hypermethylation markers are listed in Table 1. In certain embodiments, DNA hypermethylation markers include, without limitation, DNA sequences having SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10 and combinations thereof. In another embodiment, the DNA hypermethylation markers which are frequently methylated in grade I brain tumors, including EBF1, include DNA sequences having SEQ ID NO. 2, SEQ ID NO. 11, SEQ ID NO. 12, SEQ ID NO. 13, SEQ ID NO. 14, SEQ ID NO. 15, SEQ ID NO. 16, SEQ ID NO. 17, and combinations thereof (Table 2).

In certain embodiments, a DNA hypermethylation marker comprises adjacent CpG islands (“CpG clusters”) that are frequently methylated in brain cancer. In certain embodiment, the DNA hypermethylation marker comprises five or more adjacent CpG islands that underwent frequent simultaneous hypermethylation in brain cancer but are not frequently methylated in normal brains. In certain embodiments, the brain cancer is a brain tumor (e.g. a grade I, grade II, grade III or grade IV tumor). In certain embodiments, the DNA hypermethylation marker comprises CpG clusters associated with homeobox genes. In certain embodiments, the DNA hypermethylation marker comprises CpG clusters that are not associated with homeobox genes. Examples of CpG clusters associated with homeobox genes include, without limitation, the HOX gene clusters HOXD (FIG. 4), HOXC (FIG. 9), HOXA, HOXB, the homeobox genes DLX1, BARHL2 and PITX2 (FIGS. 10 and 11), developmental transcription factor gene simple-minded one (SIM1) (FIG. 12), CpG clusters on chromosome 14 (FIG. 13) which comprises several homeobox and transcription factor genes including NKX2-8 PAX9 and FOXA1. Another example of CpG clusters is on chromosome 6p21 (25,700,000-30,500,000) (FIG. 5), comprising the major histone cluster 1 and several HLA genes of the MHC locus (FIG. 5).

Another aspect of the present disclosure relates to a method of diagnosing brain cancer in a human subject comprising:

providing a sample containing acellular DNA from the human subject, wherein:
the sample is selected from the group consisting of a blood sample, serum sample and plasma sample; and
   determining whether one or more DNA hypermethylation markers on the acellular DNA are hypermethylated, wherein:
the hypermethylation of the DNA hypermethylation markers is indicative of brain cancer, and
the DNA hypermethylation markers are selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, and combinations thereof.

In certain embodiments, the brain cancer diagnosed is a brain tumor. In certain embodiments, the brain tumor is a grade I to grade IV tumor. In certain embodiments, the brain tumor is a grade I tumor. In certain embodiments, the DNA hypermethylation markers are selected from the group consisting of SEQ ID NO. 2, SEQ ID NO. 11, SEQ ID NO. 12, SEQ ID NO. 13, SEQ ID NO. 14, SEQ ID NO. 15, SEQ ID NO. 16, SEQ ID NO. 17, and combinations thereof.

The present disclosure provides a global view of DNA hypermethylation events occurring in human astrocytomas. We determined that 6,000 to 7,000 CpG islands are methylated in normal brain. This number represents well over 20% of all CpG islands in total and ˜5% of all CpG islands at 5′ gene ends were methylated. We found that genes with methylation peaks at their 5′ end are expressed at significantly lower level than genes without methylation peaks at the 5′ end.

One important attribute of a diagnostic for brain cancer is that the DNA hypermethylation markers are that they should be methylated in nearly all tumors, including early stage and low-grade tumors. To accelerate biomarker development, we created a list of 428 methylated regions that were methylated in >25% of tumors (i.e. in at least 8 of the 30 tumors analyzed) (Table 1). The most frequently methylated region was an uncharacterized CpG island on chromosome 7 that is methylated in 87% of the tumors (SEQ ID NO. 1: Chromosome 7, starting from 152252636 to 152253475). The most frequently methylated gene-associated region was within the gene body of the EBF1 gene located on chromosome 5 (SEQ ID NO. 2: Chromosome 5, starting from 158456493 to 158457132). The second most frequently methylated gene was DHH (Desert hedgehog), methylated in 83% of the tumors (SEQ ID NO. 3: Chromosome 12, starting from 47771091 to 47771619). We identified methylated regions that were methylation-positive in at least 5 of the 6 grade I tumors, including EBF1 (Table 2).

One somewhat unexpected result of our study was the finding that grade IV tumors have fewer hypermethylated regions than several of the grade II and III tumors.

Several known tumor suppressor genes are methylated in astrocytomas. These include the genes CDKN2A, PTEN, p14ARF, and RB. The reason that these genes are not listed in Tables 1 and 5 are for the most part that these genes were not methylated in more than 25% of the tumors (our criterion to be included on the list). This finding is consistent with the relatively infrequent methylation of these genes reported in the literature. We found methylation of the PTEN promoter in 20% of the tumors. CDKN2A was methylated in 23% and two tumors displayed no signal in the entire gene area suggesting homozygous deletion. For the MGMT gene, for which reported methylation frequencies in gliomas generally range from 40% to over 70%, we found weak signals at the promoter in normal brain and enhanced signals in about 40% of the tumors. However, the statistical cutoff used to score differential peaks (see Methods) did not allow this gene to be scored on our list. On the list of the 288 most frequently methylated genes, homeobox genes and other developmental regulatory transcription factors were greatly overrepresented. Thirty-one percent of these genes were targets of the Polycomb complex (H3K27me3-marked) in embryonic stem cells (FIG. 8).

One important finding of our studies was the frequent occurrence of clusters of adjacent hypermethylated CpG islands. Several examples of these included homeobox genes. Mechanistically, this phenomenon is likely related to occupancy of larger DNA sequence stretches by the Polycomb complex surrounding homeobox genes, and would, again, reflect Polycomb-targeted methylation but in a more large-scale fashion. We also identified another situation in which multiple adjacent CpG islands became hypermethylated in tumors, as a result of being present as part of multi-copy gene families. The two examples observed, reflecting extensive hypermethylation of the NBPF gene family and of CpG islands in histone cluster 1 and HLA genes in the MHC cluster suggest that it is gene duplication events per se that may trigger DNA hypermethylation. Paradoxically, this situation is the opposite of what we have observed in lung squamous cell carcinomas in which segmental duplications are generally hypomethylated in tumors (22). These data suggest that different types of malignancies have different aberrations concerning methylation of duplicated gene sequences.

Gene ontology analysis indicated that a significantly enriched gene category with common 5′ gene end methylation included genes involved in brain development and neurogenesis, once again with frequent involvement of Polycomb target and homeobox genes (Table 3). Recent mouse models have shown that gliomas arise from neural stem cells in the subventricular zone (SVZ) and/or transit-amplifying progenitors rather than from normal glial progenitors or oligodendrocytes (26, 27). Neural stem cells in the adult SVZ give rise to highly proliferating transit-amplifying progenitor cells, which then differentiate into the different lineages, including neuroblast, astrocyte and oligodendrocyte precursors. Aberrant or failed neurogenesis, due to methylation-induced permanent silencing of neuronal differentiation genes such as POU4F3, GDNF, OTX2, NEFM, CNTN4, OTP, SIM1, FYN, EN1, CHAT, GSX2, NKX6-1, PAX6, RAX, DLX2, and BMP4 and several others (Table 3) may be a fundamental component of glioma initiation from neural stem cells (see model in FIG. 15). For example, BMP4 has been shown to reduce the glioma-initiating cell pool in glioblastomas (28). Given that SVZ neural stem cells are the cells of origin of gliomas (26, 27), these methylation changes do not simply reflect lineage committed oligodendrocyte progenitors as glioma precursors, in which such genes are likely methylation-silenced. Rather, aberrant methylation of a group of genes involved in neuronal differentiation, in cooperation with other oncogenic events such as TP53 mutation, may shift the balance from regulated differentiation towards cell proliferation and gliomagenesis in neural stem cells. The permanent silencing of pro-differentiation genes may lead to the accumulation of a cell population unable to differentiate that can persist long enough, perhaps over the lifetime of an individual, to acquire the necessary transforming genetic or epigenetic aberrations. Our data suggest that epigenetic differentiation therapy might provide a useful approach for the management of this lethal disease.

### Tissue and DNA Samples.

Six normal human brain tissues from accident victims were obtained from Capital Biosciences (Gaithersburg, Md.) and BioChain (Hayward, Calif.). Twenty-four brain tumor tissues from astrocytomas (World Health Organization grade I-IV) were obtained on Institutional Review Board approved protocols from randomly selected patients who had brain tumor surgery at the Department of Neurosurgery at the University Hospital in Dresden during 1997-2007. Histological diagnosis was performed by a local neuropathologist and confirmed by reference neuropathology. These tumors are labeled T1-(1-6), T2-(1-6), T3-(1-6), and T4-(1-6), where the number immediately following the T reflects tumor grade. Six tumors labeled T2-7, T3-(7-9), and T4-(7-8) were astrocytomas obtained from Asterand (Detroit, Mich.). No patient had chemotherapy before tissue sampling.

### Methylated-CpG Island Recovery Assay (MIRA) and Microarray Hybridization.

Tumor and normal brain DNA was fragmented by sonication to ˜500 bp average size on agarose gels. Enrichment of the methylated double-stranded DNA fraction by MIRA was performed as described previously (18, 19). The labeling of amplicons, microarray hybridization, and scanning were performed according to the NimbleGen protocol. NimbleGen tiling arrays were used for hybridization (385K Human CpG Island plus Promoter arrays). The single array design covers all 28,226 UCSC Genome Browser-annotated CpG islands and the promoter regions for all RefSeq genes. The promoter region covered is one kilobase (−800 to +200 relative to the transcription start sites). For all samples, the MIRA enriched DNA was compared with the input DNA.

### Identification and Annotation of Methylated Regions.

Log 2 ratio data were converted into P value scores using the Kolmogorov-Smirnov (KS) test with a 750 bp window by using the NimbleScan software (Sp=−log 10(p)). Probes were selected as positive if their P value scores were above 2 (P<0.01). For our analysis, we defined a methylated region of interest (methylation peak) as a region with at least 4 consecutive positive probes (i.e. no gaps) covering a minimum length of 350 bp. This stringent definition will give few false-positive results. Identified methylation peaks were mapped relative to known transcripts defined in the UCSC Genome Browser HG18 RefSeq database. Methylation peaks falling into 1000 bp relative to transcription start sites were defined as 5′-end peaks; methylation peaks falling within 1000 bp of RefSeq transcript end sites were defined as 3′-end peaks, and those falling within gene bodies (from 1000 bp downstream of transcription start to 1000 bp upstream of transcript end) were defined as “intragenic” peaks. Methylation peaks that are not close to any known transcripts were defined as “intergenic.”

### Identification of Tumor-Specific Methylated Regions

Methylation peaks in tumor samples were identified as above, and methylation peaks in normal samples were identified using a P value score >=1.7 to exclude regions with weak methylation. The methylated regions in tumor and normal samples were matched by outer join and their methylation status was determined by the presence of methylation peaks in each sample in these matched regions. Only the regions methylated in more than 25% of the 30 tumors and unmethylated in all of the six normal samples were considered as candidates. The average P value scores of probes within these candidates in tumor samples were compared to the average P value scores of probes in normal samples, and the final tumor-specific methylation peaks were selected if the difference was more than 1. The tumor specific methylation peaks were also mapped to the 28,226 CpG islands defined as such at the UCSC Genome Browser. The CpG islands are defined as having a G/C content of >50%, CpG observed to expected ratio of >0.6 and a length of at least 200 bp. These CpG islands were also mapped relative to known transcripts in the HG18 RefSeq database using the same approach described above. The data reported in this paper have been deposited in the National Center for Biotechnology Information Gene Expression Omnibus (GEO) database (accession no. GSE19391).

### DNA Methylation Analysis Using Sodium Bisulfite-Based Methods.

DNA was treated and purified with the EpiTect Bisulfite kit (Qiagen; Valencia, Calif.). PCR primer sequences for amplification of specific gene targets in bisulfitetreated DNA are available upon request. The PCR products were analyzed by combined bisulfite restriction analysis (COBRA) as described previously (20). Additionally, PCR products from bisulfite-converted DNA were cloned into the pDrive PCR cloning vector (Qiagen), and 8-12 individual clones were sequenced.

### Analysis of DNA Methylation in Normal Brain.

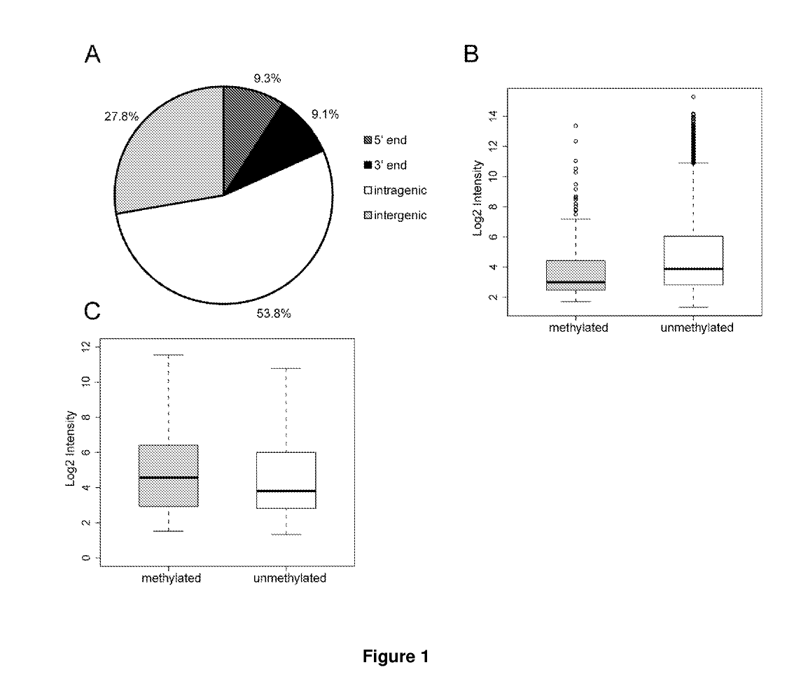

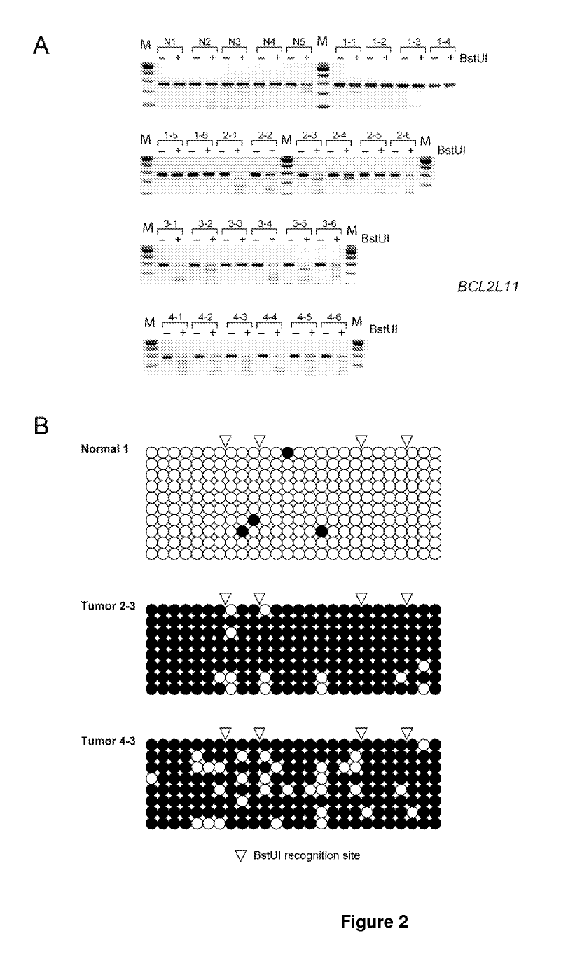

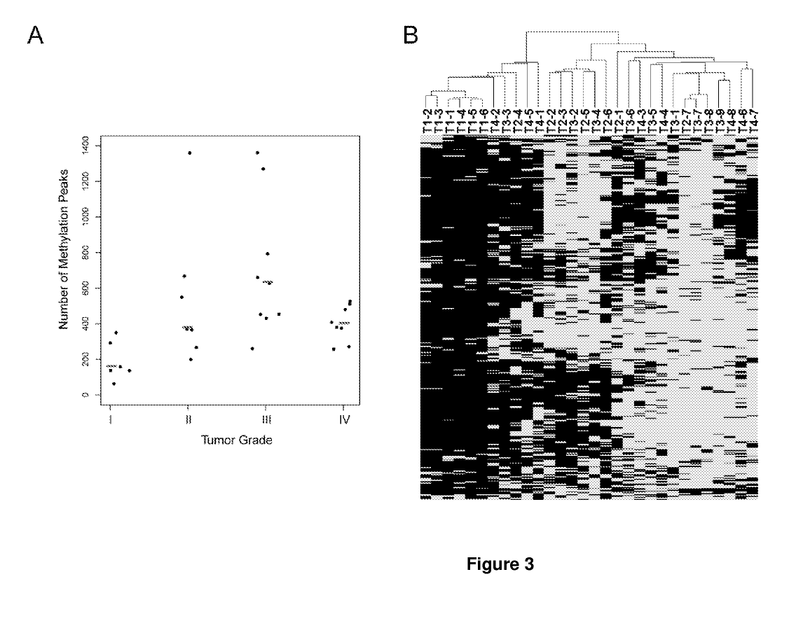

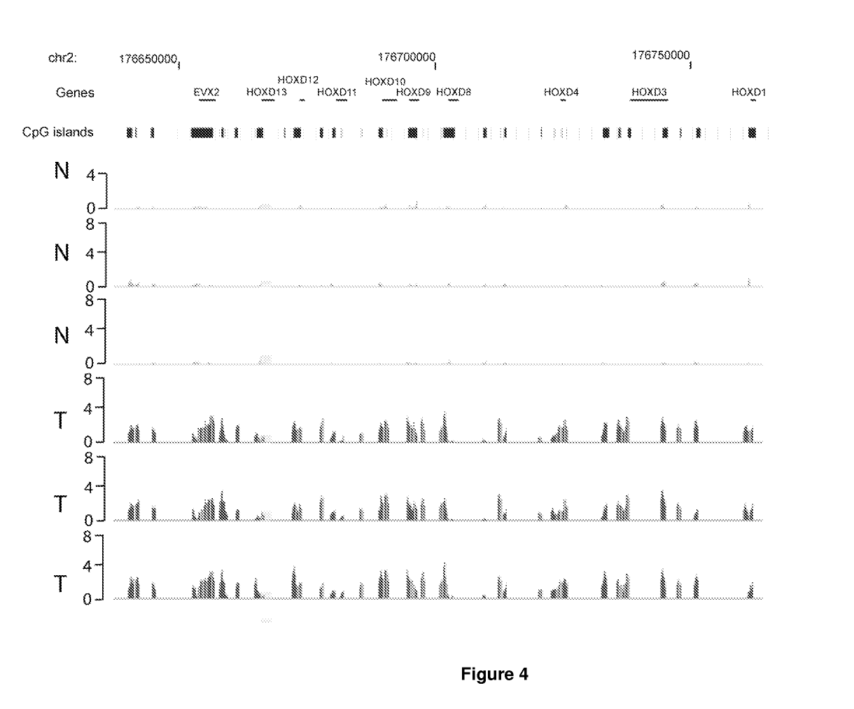

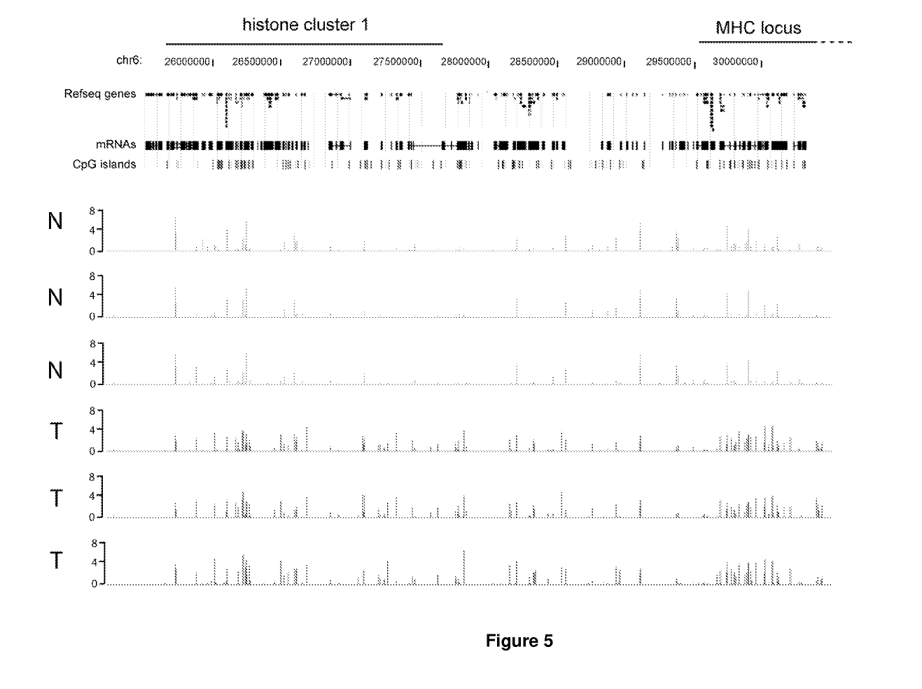

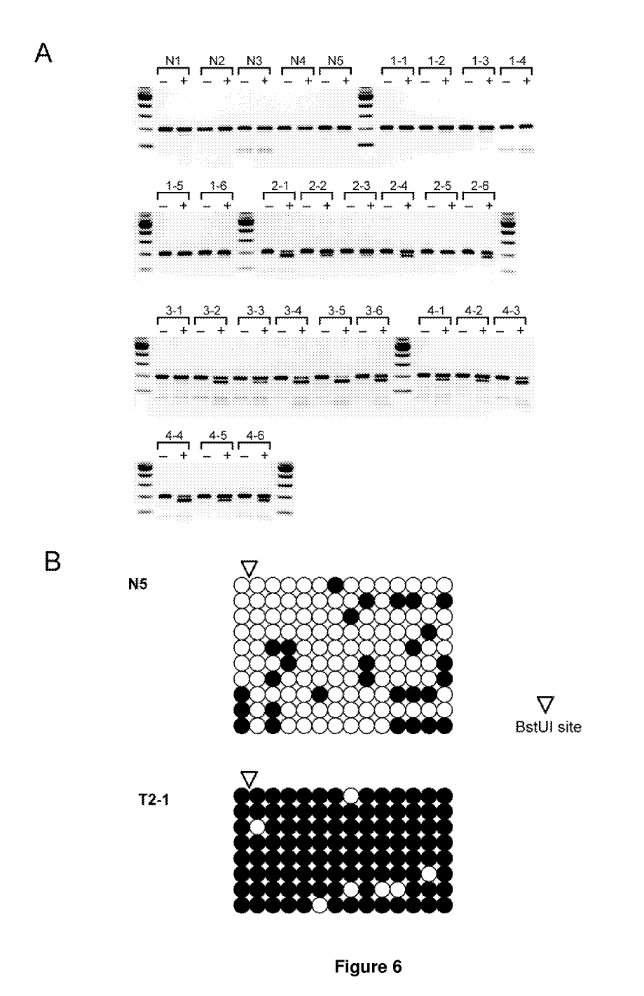

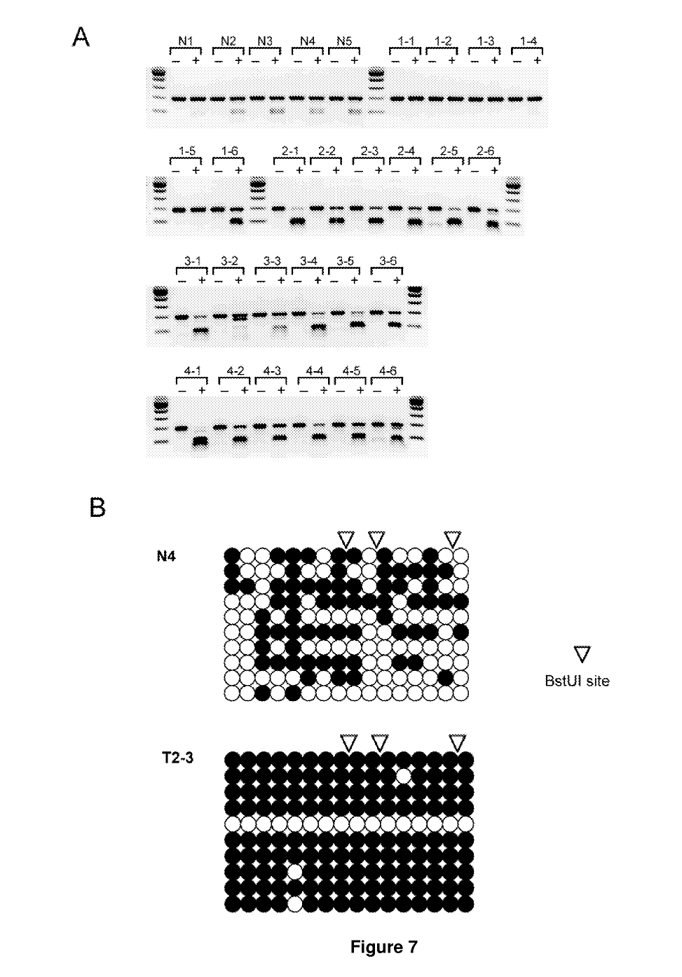

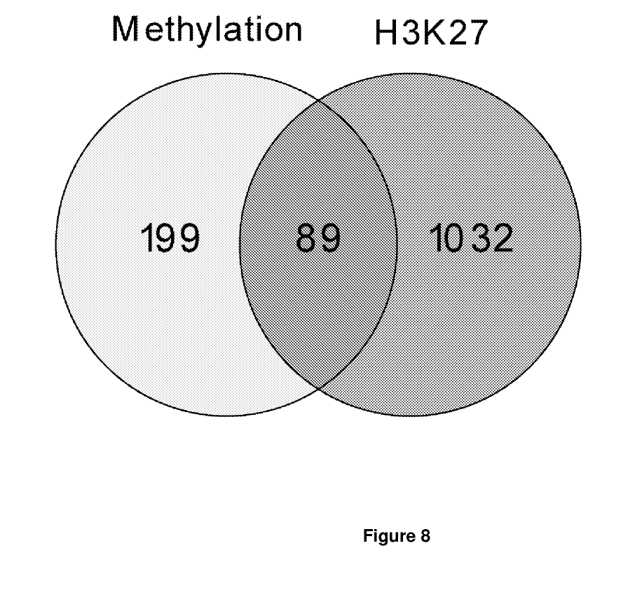

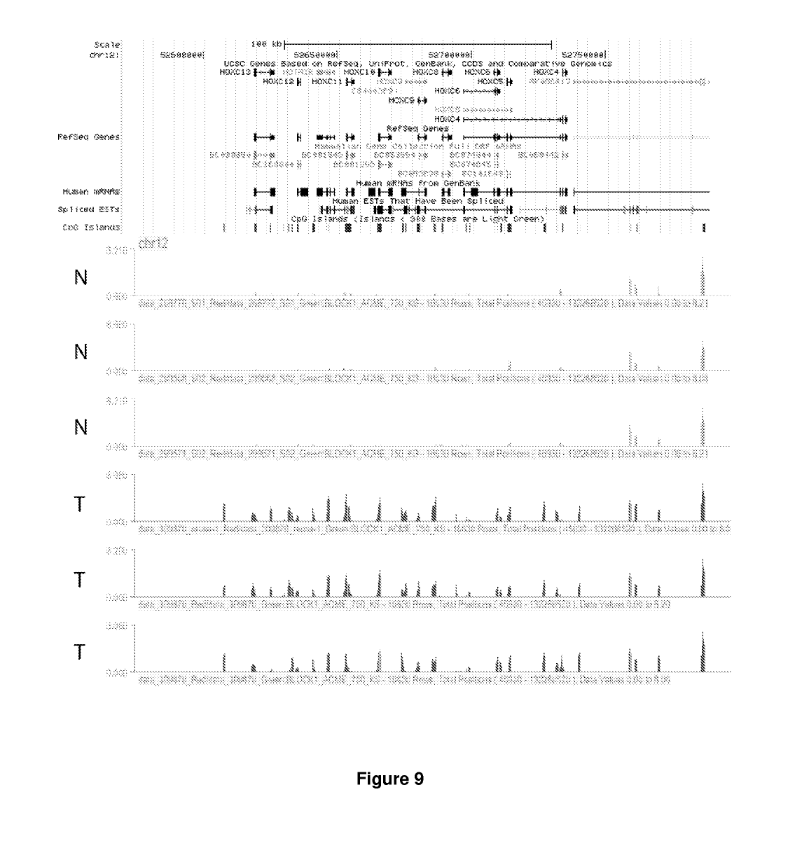

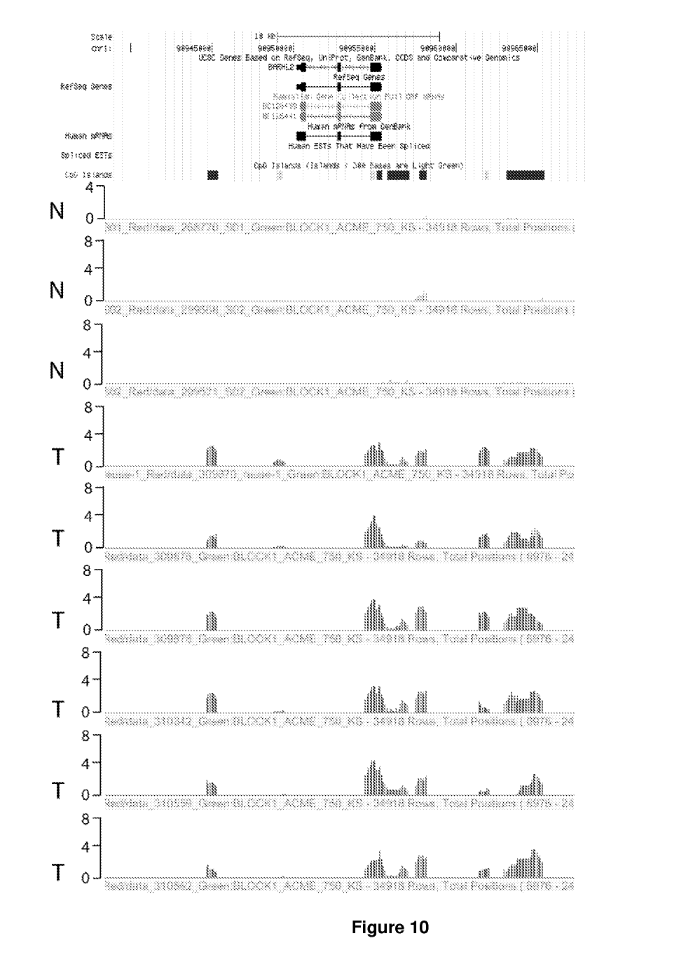

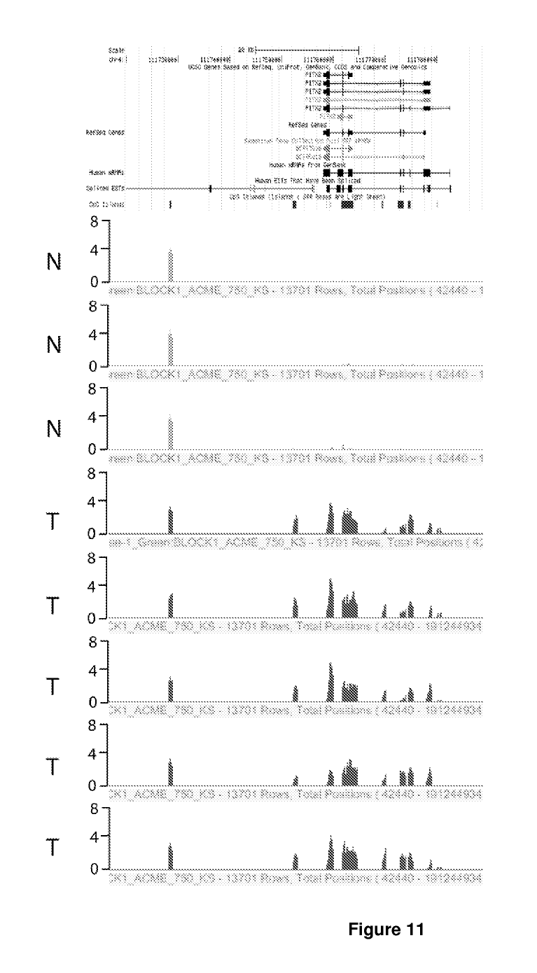

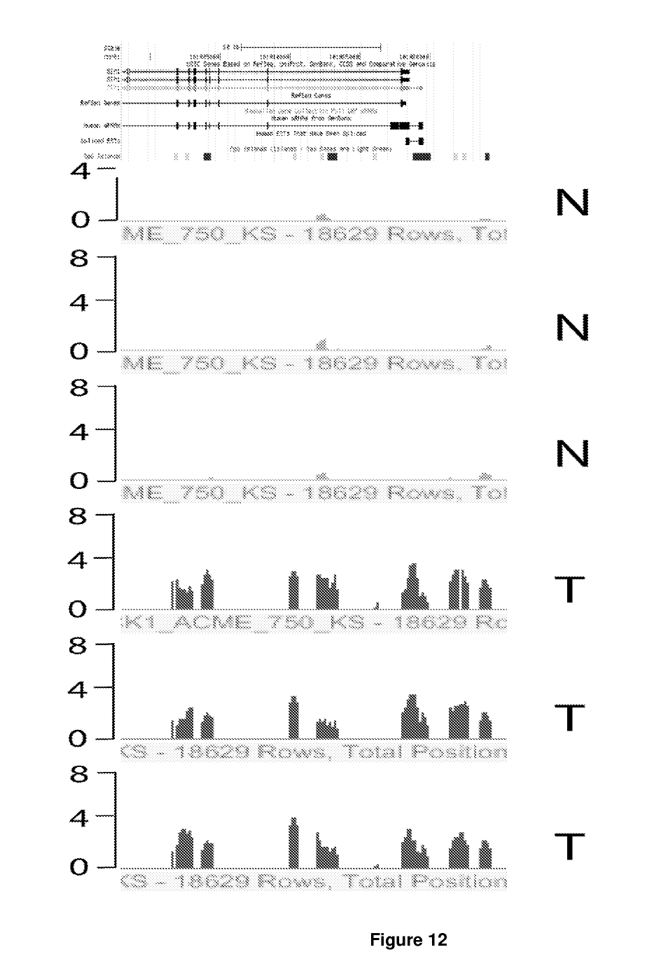

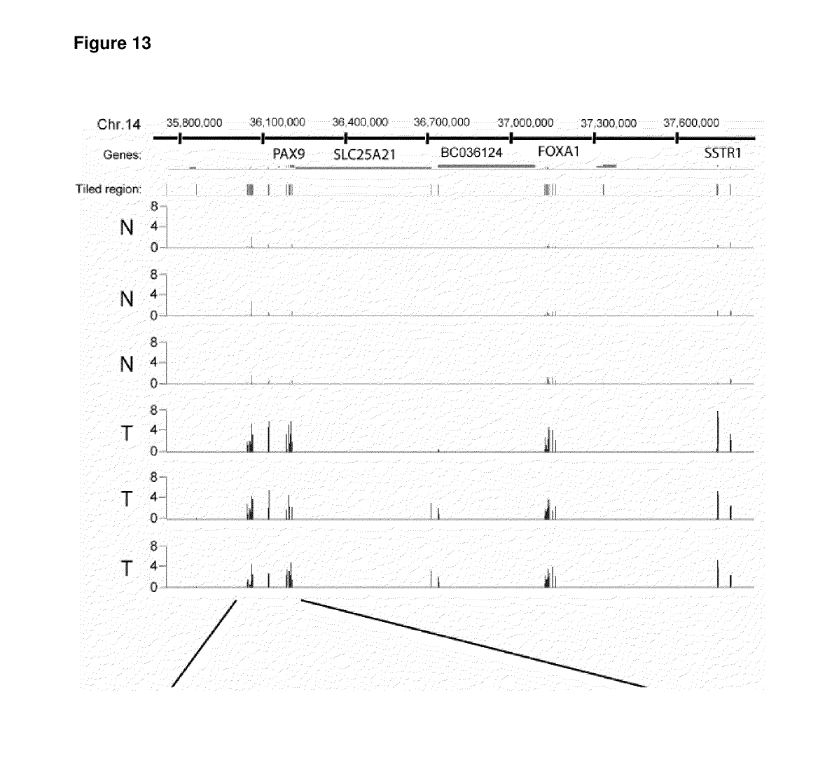

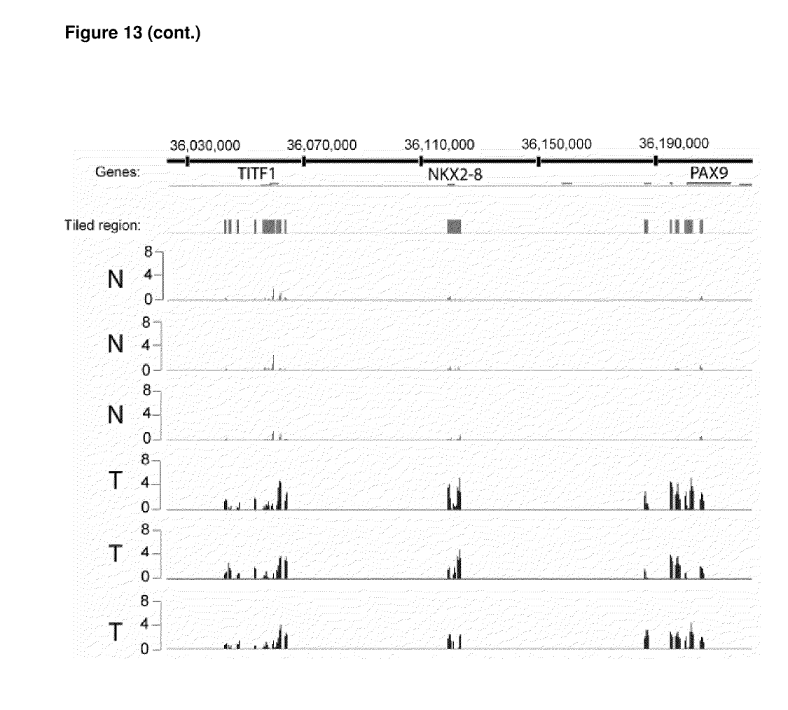

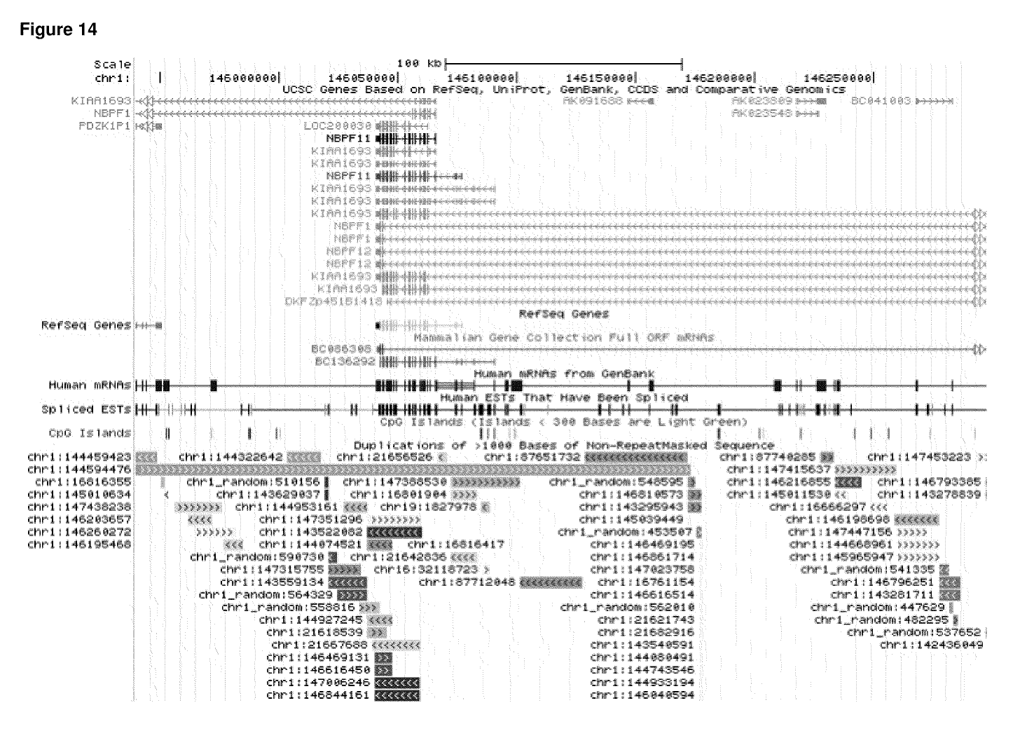

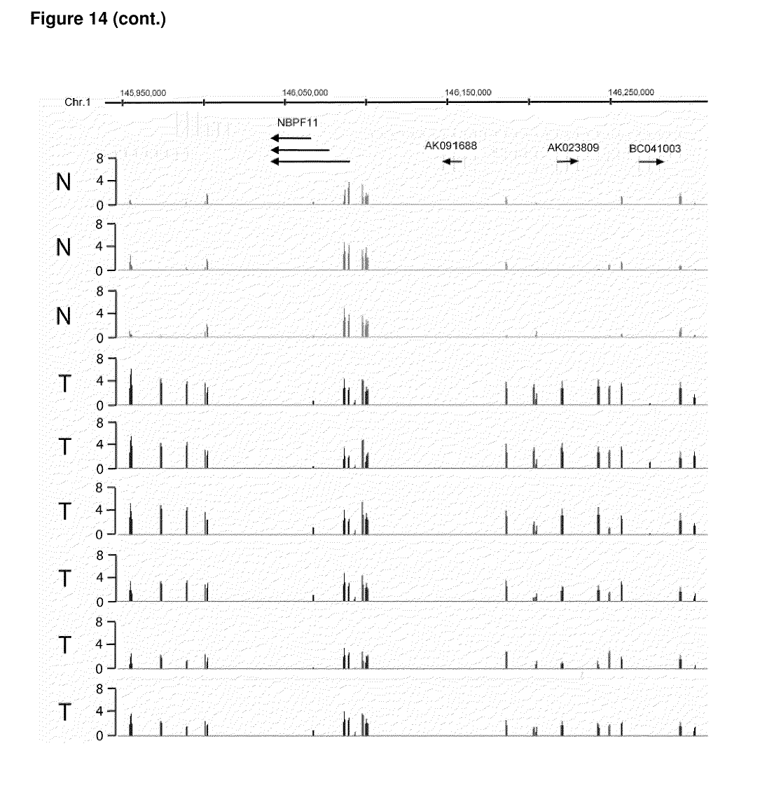

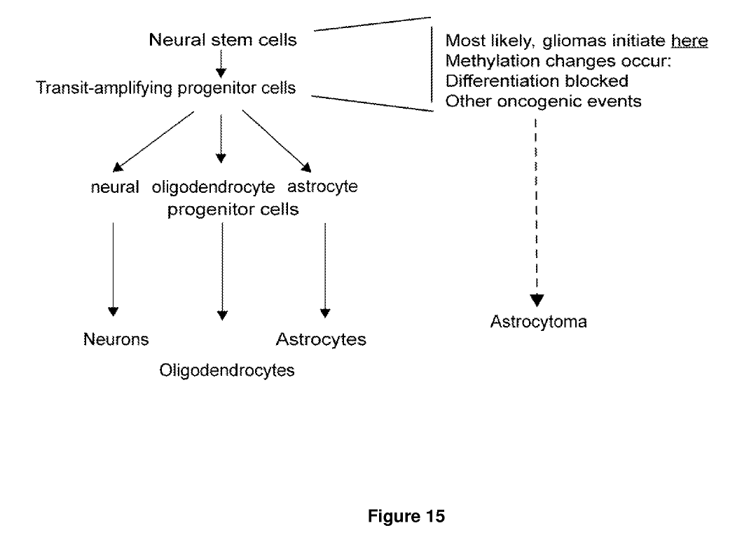

MIRA-chip has proven to be a sensitive, robust and reproducible technique for mapping DNA methylation patterns in mammalian genomes including cancer tissues (12, 14, 19). Six normal brain tissue DNA samples from accident victims were used to determine patterns of methylation at CpG islands and promoters using NimbleGen tiling arrays. After enrichment of the methylated DNA fractions by MIRA, MIRA and input fractions were mixed and hybridized to the arrays, and data were normalized and analyzed as described in Materials and Methods. We found a surprisingly large number of methylated CpG islands in normal human brain ranging from 6,000 to 7,000 in each sample. These numbers correspond to approximately 21-25% of all CpG islands as defined above. We then analyzed the localization of these methylation peaks relative to known genes. We found that 9.3% of the methylation peaks were at the 5′ ends of genes (defined as +/−1000 bp relative to transcription start sites), 53.8% were within gene bodies (defined as within 1000 bp downstream of the transcription start site and 1000 bp upstream of the gene end of RefSeq genes), and 9.1% were near the 3′ end of genes (defined as +/−1000 bp of the gene end). 27.8% of the methylation peaks were not associated with genes (FIG. 1A).

Next, we focused on those genes with methylation peaks at the 5′ end and explored the relationship of this 5′ gene end methylation and gene expression patterns using public microarray gene expression data of whole brain tissue from the GNF SymAltas database. There were 456 genes with 5′-end methylation in all normal samples (Table 4). We found that genes with methylation peaks at their 5′ end are generally expressed at lower levels than genes without methylation peaks at the 5′ end, and the difference is highly significant (Kolmogorov-Smirnov Test P value=1.8e-10) (FIG. 1B). Using the Database for Annotation, Visualization and Integrated Discovery (DAVID, version 6), we obtained annotation information for 377 of these genes. Functional annotation analysis showed that genes involved in spermatogenesis are highly enriched (Benjamini-corrected P value=7.28e-04). No other gene ontology categories had corrected P values <0.05.

We then looked at the genes with methylation peaks within the gene body. There were 1768 genes with gene body methylation and without 5′-end methylation. Consistent with previous findings in B cells (21, 19), we observed that genes with gene body methylation are generally expressed at higher levels than genes without gene body methylation (FIG. 1C).

### Analysis of DNA Methylation in Brain Tumors Shows Frequent Methylation of Genes Involved in Neuronal Differentiation.

In order to characterize methylation patterns in human brain tumors, we performed MIRA-chip analysis on 30 brain tumors including six grade I, seven grade II, nine grade III, and eight grade IV astrocytomas (glioblastomas). To confirm that the microarray data correctly reflect true methylation differences, we analyzed several genes including BCL2L11, RAX, FOXP4, and ZNF274 bp sodium bisulfite-based DNA methylation analysis techniques. Consistent with previous data demonstrating the reliability of the MIRA assay (12, 14, 22, 19), we confirmed methylation differences between normal and tumor tissues by COBRA analysis and by bisulfite sequencing, as shown for the pro-apoptotic gene BCL2L11 also known as BIM (FIG. 2), for the RAX and ZNF274 genes (FIGS. 6 and 7) and for FOXP4. The BCL2L11 gene was methylated in 21 of the 30 brain tumors and mostly in tumors of grade II and higher (FIG. 2).

We catalogued those genes that are methylated in the brain tumors and defined a tumor-specifically methylated gene as one that was not methylated in any of the six normal brain tissue DNAs. Using these criteria, we determined the number of tumor-methylated regions in each tumor as shown in FIG. 3A. The number of tumor-specifically methylated regions increased in astrocytomas going from grade Ito grade III. However, this number, on average, decreased in grade IV tumors relative to grade II and III tumors. Four of the normal brain tissues available to us were from the frontal lobe, one was from the occipital lobe and one from an unspecified region of the brain. Among these six normal brain tissues, there were very few differences in terms of number and location of methylated CpG islands. Astrocytomas came from several different regions of the brain including frontal, parietal, occipital, fronto-temporal, and temporal lobes, and three of the stage I tumors were from the cerebellum. When looking at location vs. number of methylation targets adjusting for tumor stage, there was no significant correlation, but the numbers of tumors are too small to exclude the possibility of brain region-specific differences in tumor-associated methylation. We then established a list of genes that are most commonly methylated across different tumor specimens. These genes are unmethylated, i.e. lack a peak on the arrays in all six normal brain samples, and are methylated in at least 25% of the tumors (i.e. in at least 8 out of 30 tumors). We compiled a list of 428 methylation peaks that fit these criteria (Table 1). A hierarchical clustering of these tumor-specific methylated regions is shown in FIG. 3B. Grade I tumors form a tight cluster, adjacent to 5 higher grade tumors, T4-2, T3-3, T2-4, T4-5, and T4-1. The other higher-grade tumors form two distinct clusters. One cluster includes T2-2, T2-3, T3-2, T2-5, T3-4 and T2-6. The rest of the tumors form another cluster and they have the most methylation peaks. Of the 428 frequently methylated regions, 403 overlap with CpG islands, and 288 fall into genes, i.e. they are within 1000 bp upstream of the transcription start site and 1000 bp downstream of the transcription end of a gene. There were 174 methylation peaks that can be annotated to 5′-ends of Refseq transcripts (Table 5). Of these, 164 genes had annotation information in DAVID. Functional analysis revealed that genes involved in organ, nervous system and brain development, and neurogenesis are highly enriched (Table 3). Many of these genes are transcription factor and homeobox genes. An analysis of Interpro domains revealed 35 homeobox genes in the list of 288 genes with methylation peaks (Enrichment-fold=12.2, P=3.7e-26, InterPro domain IPR001356). Since homeobox genes and other developmental regulatory genes are known targets of the repressive Polycomb complex in embryonic stem (ES) cells, we compared the list of commonly methylated genes with the list of genes marked as Polycomb targets in human ES cells (23). FIG. 8 shows that indeed 30.7% of the methylated genes are Polycomb targets. The observed number of overlapping targets between MIRA and Polycomb is more than 7-fold higher than expected by chance, indicating a strong enrichment (P<2.2e-16; Fisher's exact test).

Although we focused mostly on DNA hypermethylation in this study, we also looked for hypomethylation of CpG islands in the tumor samples relative to normal brain. We identified a list of 46 genes where the hypomethylation event is frequently present at the 5′ gene end (Table 6). This list was enriched for cancer-testis antigen genes including the genes SOHLH2, SSX2, SSX4B, SSX8, SSX9, and PAGES. This class of genes is normally expressed in germ cells, is silenced by DNA methylation in somatic tissues but can be reactivated and demethylated in cancer.

### Clusters of CpG Island Hypermethylation in Brain Tumors.

In addition to the many singular hypermethylated CpG islands present along all chromosomes, we identified a number of prominent clusters of hypermethylated CpG islands. These clusters were defined as a minimum of five adjacent CpG islands that underwent simultaneous hypermethylation in at least five of the tumors but are methylation-free in all normal brains. These clusters could be divided into two categories: (1) CpG islands associated with homeobox genes and (2) hypermethylation clusters not associated with homeobox genes. Category 1 included the following loci: the HOX gene clusters HOXD (FIG. 4) and HOXC (FIG. 9), and the homeobox genes DLX1, BARHL2 and PITX2 (FIGS. 10 and 11), as well as the developmental transcription factor gene simple-minded one (SIM1) (FIG. 12). The HOX clusters HOXA and HOXB were also hypermethylated in tumors but several of the genes in these two clusters had considerable methylation in normal brain. The hypermethylated CpG islands at these homeobox gene loci were found not only at the 5′ gene ends but also within the gene bodies and at the 3′ ends. Chromosome 14 shows a particularly illustrative example of homeobox gene-associated hypermethylation of CpG island clusters (FIG. 13). In this case, several homeobox and transcription factor genes, including NKX2-8, PAX9, and FOXA1 are located within a 1.8 Mb segment, and no less than 24 contiguous CpG islands become hypermethylated in astrocytomas. A 360 kilobase stretch at chromosome 1q21.2 (145,940,000-146,300,000) contained 12 hypermethylated CpG islands. In this case, four consecutive tumor-specifically hypermethylated islands and eight adjacent tumorspecifically methylated islands were separated by two CpG islands that were methylated in tumors and in normal brain. Interestingly, this chromosomal region is rich in segmental duplications (FIG. 14). This locus contains the neuroblastoma breakpoint family (NBPF) of genes named after a chromosomal translocation found in a neuroblastoma patient (24). The NBPF genes encode proteins of unknown function and have a repetitive structure with high intragenic and intergenic sequence similarity in both coding and noncoding regions. No discernable NBPF orthologues have been identified in the genomes of mouse or rat. This gene family shows primate-specific duplications that result in species-specific arrays of NBPF homologous sequences (24). The hypermethylation cluster on chromosome 6p21 (25,700,000-30,500,000) spanning 4.8 megabases is particularly striking. This hypermethylation cluster was present in 16 out of 30 tumors analyzed (FIG. 5). The region contains the major histone cluster 1 and several HLA genes of the MHC locus. This large cluster of histone genes contains 55 histone genes (25). Thus, this chromosomal region represents a segment where closely related genes, the histone gene families and HLA genes are present and are subject to hypermethylation in brain tumors. Although some CpG islands in this region are methylated in normal brain as well, there are 42 CpG islands that are subject to tumor-specific hypermethylation.

### REFERENCE

All the references listed below are incorporated herein by reference:
1. Alcantara Llaguno S R, Chen J, Parada L F. Signaling in malignant astrocytomas: role of neural stem cells and its therapeutic implications. Clin Cancer Res 2009; 15:7124-9.
2. Parsons D W, Jones S, Zhang X, et al. An integrated genomic analysis of human glioblastoma multiforme. Science 2008; 321:1807-12.
3. Jones P A, Baylin S B. The epigenomics of cancer. Cell 2007; 128:683-92.
4. Nagarajan R P, Costello J F. Epigenetic mechanisms in glioblastoma multiforme. Semin Cancer Biol 2009; 19:188-97.
5. Costello J F, Fruhwald M C, Smiraglia D J, et al. Aberrant CpG-island methylation has non-random and tumour-type-specific patterns. Nat Genet 2000; 24:132-8.
6. Uhlmann K, Rohde K, Zeller C, et al. Distinct methylation profiles of glioma subtypes. Int J Cancer 2003; 106:52-9.
7. Yu J, Zhang H, Gu J, et al. Methylation profiles of thirty four promoter-CpG islands and concordant methylation behaviours of sixteen genes that may contribute to carcinogenesis of astrocytoma. BMC Cancer 2004; 4:65.
8. Cadieux B, Ching T T, VandenBerg S R, Costello J F. Genome-wide hypomethylation in human glioblastomas associated with specific copy number alteration, methylenetetrahydrofolate reductase allele status, and increased proliferation. Cancer Res 2006; 66:8469-76.
9. Foltz G, Ryu G Y, Yoon J G, et al. Genome-wide analysis of epigenetic silencing identifies BEX1 and BEX2 as candidate tumor suppressor genes in malignant glioma. Cancer Res 2006; 66:6665-74.
10. Kim T Y, Zhong S, Fields C R, Kim J H, Robertson K D. Epigenomic profiling reveals novel and frequent targets of aberrant DNA methylation-mediated silencing in malignant glioma. Cancer Res 2006; 66:7490-501.
11. Fanelli M, Caprodossi S, Ricci-Vitiani L, et al. Loss of pericentromeric DNA methylation pattern in human glioblastoma is associated with altered DNA methyltransferases expression and involves the stem cell compartment. Oncogene 2008; 27:358-65.
12. Rauch T, Li H, Wu X, Pfeifer G P. MIRA-assisted microarray analysis, a new technology for the determination of DNA methylation patterns, identifies frequent methylation of homeodomain-containing genes in lung cancer cells. Cancer Res 2006; 66:7939-47.
13. Ohm J E, McGarvey K M, Yu X, et al. A stem cell-like chromatin pattern may predispose tumor suppressor genes to DNA hypermethylation and heritable silencing. Nat Genet 2007; 39:237-42.
14. Rauch T, Wang Z, Zhang X, et al. Homeobox gene methylation in lung cancer studied by genome-wide analysis with a microarray-based methylated CpG island recovery assay. Proc Natl Acad Sci USA 2007; 104:5527-32.
15. Schlesinger Y, Straussman R, Keshet I, et al. Polycomb-mediated methylation on Lys27 of histone H3 pre-marks genes for de novo methylation in cancer. Nat Genet 2007; 39:232-6.
16. Widschwendter M, Fiegl H, Egle D, et al. Epigenetic stem cell signature in cancer.

Nat Genet 2007; 39:157-8.
17. Gal-Yam E N, Egger G, lniguez L, et al. Frequent switching of Polycomb repressive marks and DNA hypermethylation in the PC3 prostate cancer cell line. Proc Natl Acad Sci USA 2008; 105:12979-84.
18. Rauch T A, Pfeifer G P. The MIRA method for DNA methylation analysis. Methods Mol Biol 2009; 507:65-75.
19. Rauch T A, Wu X, Zhong X, Riggs A D, Pfeifer G P. A human B cell methylome at 100-base pair resolution. Proc Natl Acad Sci USA 2009; 106:671-8.
20. Xiong Z, Laird P W. COBRA: a sensitive and quantitative DNA methylation assay. Nucleic Acids Res 1997; 25:2532-4.
21. Ball M P, Li J B, Gao Y, et al. Targeted and genome-scale strategies reveal gene-body methylation signatures in human cells. Nat Biotechnol 2009; 27:361-8.
22. Rauch T A, Zhong X, Wu X, et al. High-resolution mapping of DNA hypermethylation and hypomethylation in lung cancer. Proc Natl Acad Sci USA 2008; 105:252-7.
23. Lee T I, Jenner R G, Boyer L A, et al. Control of developmental regulators by Polycomb in human embryonic stem cells. Cell 2006; 125:301-13.
24. Vandepoele K, Van Roy N, Staes K, Speleman F, van Roy F. A novel gene family NBPF: intricate structure generated by gene duplications during primate evolution. Mol Biol Evol 2005; 22:2265-74.
25. Marzluff W F, Gongidi P, Woods K R, Jin J, Maltais L J. The human and mouse replication-dependent histone genes. Genomics 2002; 80:487-98.
26. Alcantara Llaguno S, Chen J, Kwon C H, et al. Malignant astrocytomas originate from neural stem/progenitor cells in a somatic tumor suppressor mouse model. Cancer Cell 2009; 15:45-56.
27. Wang Y, Yang J, Zheng H, et al. Expression of mutant p53 proteins implicates a lineage relationship between neural stem cells and malignant astrocytic glioma in a murine model. Cancer Cell 2009; 15:514-26.
28. Piccirillo S G, Reynolds B A, Zanetti N, et al. Bone morphogenetic proteins inhibit the tumorigenic potential of human brain tumour-initiating cells. Nature 2006; 444:761-5.

TABLE 1
Frequently methylated regions in brain tumors.

| Peaks | | | Average P | No. of Tumors | Downstream | | | Gene | Within | |
|---|---|---|---|---|---|---|---|---|---|---|
| chr | Start | End | value score * | with Peaks | Strand | Start | End | Symbol | Strand | Start |
| chr1 | 2456587 | 2457246 | 7.35 | 19 | | | | | | |
| chr1 | 37973297 | 37974046 | 12.12 | 17 | | | | | − | 37954232 |
| chr1 | 38002438 | 38003587 | 10.10 | 12 | | | | | | |
| chr1 | 46712589 | 46713323 | 7.31 | 9 | | | | | | |
| chr1 | 47469314 | 47470340 | 8.43 | 11 | | | | | | |
| chr1 | 54594420 | 54595152 | 6.92 | 14 | | | | | − | 54464782 |
| chr1 | 60311689 | 60312524 | 9.01 | 16 | | | | | | |
| chr1 | 63556597 | 63560345 | 8.49 | 15 | | | | | | |
| chr1 | 90963290 | 90965440 | 10.04 | 16 | | | | | | |
| chr1 | 111614814 | 111615476 | 11.27 | 20 | | | | | | |
| chr1 | 119328183 | 119328832 | 10.62 | 21 | | | | | − | 119227188 |
| chr1 | 143012194 | 143012944 | 10.97 | 14 | − | 143011875 | 143053112 | LOC728875 | − | 143011875 |
| chr1 | 144674586 | 144675239 | 11.02 | 17 | | | | | | |
| chr1 | 144690585 | 144691329 | 9.13 | 17 | | | | | | |
| chr1 | 144702031 | 144702576 | 7.97 | 15 | | | | | | |
| chr1 | 145971587 | 145972240 | 11.05 | 19 | | | | | | |
| chr1 | 145987585 | 145988329 | 9.59 | 19 | | | | | | |
| chr1 | 145999042 | 145999587 | 8.90 | 16 | | | | | | |
| chr1 | 146184793 | 146185549 | 8.70 | 19 | | | | | | |
| chr1 | 146219303 | 146219957 | 9.13 | 12 | | | | | | |
| chr1 | 146248547 | 146249292 | 7.11 | 12 | | | | | | |
| chr1 | 146256160 | 146256719 | 8.21 | 16 | | | | | | |
| chr1 | 146466570 | 146467421 | 9.58 | 15 | | | | | | |
| chr1 | 147599481 | 147600124 | 13.18 | 21 | | | | | | |
| chr1 | 147600226 | 147600785 | 12.95 | 17 | | | | | | |
| chr1 | 147665315 | 147665969 | 7.78 | 12 | | | | | | |
| chr1 | 147882409 | 147883136 | 6.55 | 15 | + | 147843076 | 147883408 | LOC728855 | | |
| chr1 | 147985011 | 147986318 | 13.04 | 21 | | | | | | |
| chr1 | 150078663 | 150079332 | 11.19 | 11 | | | | | + | 150077568 |
| chr1 | 150349668 | 150350912 | 10.81 | 12 | | | | | − | 150345416 |
| chr1 | 153320294 | 153321238 | 8.12 | 11 | | | | | + | 153317971 |
| chr1 | 153557083 | 153557827 | 8.77 | 11 | − | 153556874 | 153560562 | C1orf104 | | |
| chr1 | 154527610 | 154528254 | 8.01 | 14 | − | 154529107 | 154532073 | C1orf85 | + | 154520693 |
| chr1 | 155085184 | 155085851 | 7.40 | 16 | | | | | − | 155077288 |
| chr1 | 165119820 | 165120544 | 12.95 | 12 | | | | | | |
| chr1 | 179719350 | 179719904 | 10.69 | 13 | | | | | | |
| chr1 | 181421423 | 181422187 | 9.18 | 19 | | | | | | |
| chr1 | 196156945 | 196157494 | 11.07 | 20 | | | | | + | 196153139 |
| chr1 | 207914587 | 207915953 | 6.93 | 9 | + | 207915292 | 207916358 | G0S2 | + | 207915292 |
| chr1 | 219131519 | 219132268 | 10.21 | 20 | | | | | | |
| chr1 | 219134095 | 219134834 | 6.51 | 11 | | | | | | |
| chr1 | 222002983 | 222003622 | 6.09 | 15 | | | | | + | 221966741 |
| chr1 | 222870205 | 222872650 | 9.25 | 10 | | | | | + | 222870801 |
| chr1 | 226700008 | 226700767 | 11.86 | 10 | | | | | | |
| chr1 | 234752777 | 234754328 | 9.67 | 11 | | | | | + | 234748187 |
| chr10 | 7489395 | 7490153 | 9.35 | 15 | | | | | − | 7244254 |
| chr10 | 46412477 | 46413703 | 7.66 | 21 | | | | | | |
| chr10 | 50486546 | 50487915 | 9.95 | 11 | | | | | | |
| chr10 | 50488946 | 50491715 | 10.13 | 15 | + | 50488352 | 50490772 | SLC18A3 | + | 50488352 |
| chr10 | 79066119 | 79066773 | 9.27 | 12 | | | | | − | 78314641 |
| chr10 | 81732969 | 81733640 | 6.61 | 9 | | | | | | |
| chr10 | 99070134 | 99071063 | 10.07 | 15 | + | 99069011 | 99071662 | FRAT1 | + | 99069011 |
| chr10 | 102580005 | 102580574 | 5.67 | 12 | + | 102495457 | 102579688 | PAX2 | | |
| chr10 | 102972304 | 102973663 | 11.25 | 18 | | | | | | |
| chr10 | 120344105 | 120345734 | 7.81 | 11 | | | | | − | 120342905 |
| chr10 | 124628733 | 124629607 | 7.80 | 12 | | | | | | |
| chr11 | 1718992 | 1720660 | 11.14 | 21 | | | | | − | 1447268 |
| chr11 | 1721203 | 1723357 | 8.45 | 9 | | | | | | |
| chr11 | 1727398 | 1728562 | 7.56 | 12 | | | | | | |
| chr11 | 2102906 | 2103345 | 7.82 | 16 | | | | | | |
| chr11 | 2183194 | 2184458 | 6.81 | 9 | | | | | | |
| chr11 | 10428000 | 10428654 | 7.76 | 16 | | | | | | |
| chr11 | 14883948 | 14884302 | 8.94 | 20 | | | | | | |
| chr11 | 19220281 | 19221020 | 7.43 | 18 | | | | | | |
| chr11 | 22319354 | 22320095 | 12.00 | 13 | | | | | + | 22316242 |
| chr11 | 31789500 | 31795000 | 8.20 | 10 | | | | | − | 31768059 |
| chr11 | 32411453 | 32412722 | 13.77 | 15 | | | | | − | 32365900 |
| chr11 | 44283814 | 44284478 | 7.06 | 13 | | | | | − | 44242733 |
| chr11 | 44935052 | 44935706 | 8.16 | 17 | | | | | | |
| chr11 | 63531706 | 63532455 | 8.98 | 14 | | | | | − | 63522606 |
| chr11 | 63823533 | 63825290 | 17.92 | 13 | + | 63815368 | 63824079 | KCNK4 | | |
| chr11 | 64947186 | 64947820 | 7.85 | 12 | | | | | | |
| chr11 | 73049464 | 73050318 | 8.48 | 16 | | | | | + | 73037382 |
| chr11 | 94523361 | 94524500 | 13.90 | 19 | | | | | | |
| chr11 | 124133793 | 124135432 | 6.76 | 13 | | | | | − | 124128228 |
| chr11 | 133762389 | 133763138 | 6.79 | 15 | | | | | − | 133753607 |
| chr12 | 6803351 | 6804025 | 5.33 | 12 | | | | | + | 6801223 |
| chr12 | 21985422 | 21986671 | 6.67 | 11 | | | | | | |
| chr12 | 42231009 | 42232738 | 12.35 | 17 | | | | | | |
| chr12 | 45511201 | 45511951 | 7.23 | 13 | | | | | | |
| chr12 | 47771091 | 47771619 | 9.92 | 24 | | | | | − | 47769472 |
| chr12 | 47975997 | 47977646 | 11.74 | 21 | | | | | + | 47975175 |
| chr12 | 52418168 | 52418832 | 5.91 | 8 | | | | | | |
| chr12 | 52646454 | 52647108 | 9.76 | 19 | | | | | − | 52642362 |
| chr12 | 53049097 | 53049742 | 7.62 | 18 | − | 53049186 | 53071350 | ZNF385A | | |
| chr12 | 55168271 | 55169242 | 10.27 | 19 | | | | | | |
| chr12 | 55918204 | 55919848 | 21.03 | 9 | | | | | − | 55914952 |
| chr12 | 56300983 | 56301939 | 13.42 | 12 | | | | | + | 56299959 |
| chr12 | 79626169 | 79626805 | 12.87 | 20 | + | 79625576 | 79627382 | MYF6 | + | 79625576 |
| chr12 | 84197229 | 84198765 | 8.80 | 11 | | | | | | |
| chr12 | 101868310 | 101868969 | 7.61 | 12 | | | | | | |
| chr12 | 113322635 | 113323304 | 9.71 | 14 | | | | | − | 113288284 |
| chr12 | 115800795 | 115801644 | 9.95 | 17 | | | | | − | 115783409 |
| chr13 | 27399628 | 27401487 | 14.44 | 11 | | | | | | |
| chr13 | 40928925 | 40929564 | 6.50 | 17 | | | | | | |
| chr13 | 52211145 | 52212607 | 7.69 | 14 | | | | | | |
| chr13 | 102224101 | 102224965 | 10.63 | 16 | | | | | | |
| chr13 | 112725390 | 112726034 | 8.32 | 17 | | | | | + | 112681655 |
| chr14 | 22360909 | 22361365 | 8.50 | 24 | | | | | | |
| chr14 | 23115638 | 23116287 | 7.60 | 14 | | | | | − | 23107083 |
| chr14 | 36061370 | 36062206 | 8.09 | 12 | | | | | | |
| chr14 | 36122209 | 36123483 | 8.76 | 9 | | | | | | |
| chr14 | 36194850 | 36195499 | 12.01 | 15 | | | | | | |
| chr14 | 36196539 | 36197988 | 8.90 | 13 | | | | | + | 36196532 |
| chr14 | 36201539 | 36202583 | 10.95 | 15 | | | | | + | 36196532 |
| chr14 | 37137508 | 37138866 | 5.61 | 11 | | | | | | |
| chr14 | 37748210 | 37750570 | 10.41 | 18 | | | | | + | 37746954 |
| chr14 | 53488199 | 53488653 | 10.84 | 20 | | | | | − | 53486204 |
| chr14 | 53491636 | 53492707 | 12.27 | 14 | | | | | − | 53486204 |
| chr14 | 53499835 | 53500364 | 16.29 | 12 | | | | | | |
| chr14 | 56344682 | 56346630 | 8.36 | 9 | | | | | − | 56337177 |
| chr14 | 60021741 | 60022400 | 6.81 | 12 | | | | | | |
| chr14 | 60043368 | 60044022 | 9.32 | 12 | | | | | | |
| chr14 | 60045099 | 60047942 | 9.30 | 11 | + | 60045690 | 60048278 | SIX6 | + | 60045690 |
| chr14 | 73776450 | 73777601 | 6.38 | 8 | | | | | + | 73775927 |
| chr14 | 74147083 | 74147922 | 9.41 | 19 | | | | | − | 74034638 |
| chr14 | 94304390 | 94305319 | 7.99 | 17 | − | 94304312 | 94306252 | GSC | − | 94304312 |
| chr14 | 94308930 | 94310289 | 11.73 | 18 | | | | | | |
| chr14 | 105023673 | 105024599 | 8.21 | 12 | | | | | | |
| chr15 | 43195869 | 43196818 | 10.16 | 13 | − | 43196977 | 43209349 | DUOXA1 | + | 43193815 |
| chr15 | 43783619 | 43784273 | 7.10 | 18 | | | | | | |
| chr15 | 50867775 | 50868924 | 6.92 | 18 | | | | | − | 50836644 |
| chr15 | 50873886 | 50875041 | 9.91 | 16 | | | | | | |
| chr15 | 62984637 | 62985202 | 10.84 | 19 | | | | | | |
| chr15 | 65913980 | 65915431 | 8.60 | 12 | + | 65904994 | 65913228 | LBXCOR1 | | |
| chr15 | 72207930 | 72209773 | 11.51 | 13 | | | | | + | 72208767 |
| chr15 | 74414585 | 74415136 | 7.92 | 13 | | | | | | |
| chr15 | 81112195 | 81112934 | 8.26 | 16 | | | | | − | 81009005 |
| chr15 | 82113943 | 82114802 | 6.20 | 20 | | | | | | |
| chr15 | 87755647 | 87756297 | 7.33 | 18 | | | | | | |
| chr16 | 62621 | 63380 | 5.69 | 14 | | | | | | |
| chr16 | 4977615 | 4978589 | 6.27 | 13 | | | | | + | 4948318 |
| chr16 | 19804086 | 19804635 | 8.37 | 20 | | | | | | |
| chr16 | 29703730 | 29704362 | 7.58 | 15 | | | | | | |
| chr16 | 31014172 | 31014616 | 5.57 | 13 | | | | | | |
| chr16 | 49432345 | 49433015 | 10.49 | 16 | | | | | | |
| chr16 | 49744810 | 49746252 | 9.90 | 11 | | | | | | |
| chr16 | 52873128 | 52875454 | 8.32 | 12 | − | 52874712 | 52877879 | IRX3 | | |
| chr16 | 52879121 | 52880054 | 7.79 | 14 | | | | | | |
| chr16 | 53523342 | 53524273 | 7.02 | 16 | | | | | + | 53522611 |
| chr16 | 53527819 | 53530073 | 8.62 | 11 | | | | | | |
| chr16 | 55229290 | 55229739 | 8.87 | 19 | | | | | | |
| chr16 | 55234406 | 55235250 | 6.28 | 12 | | | | | | |
| chr16 | 55254245 | 55254911 | 7.07 | 13 | | | | | | |
| chr16 | 56575924 | 56577738 | 6.59 | 17 | | | | | + | 56567839 |
| chr16 | 65747571 | 65748520 | 10.24 | 14 | | | | | − | 65745589 |
| chr17 | 1901278 | 1904018 | 9.32 | 12 | | | | | | |
| chr17 | 4385916 | 4386650 | 7.11 | 14 | | | | | + | 4348877 |
| chr17 | 4635389 | 4636518 | 8.20 | 10 | − | 4635320 | 4636469 | VMO1 | − | 4635320 |
| chr17 | 4638694 | 4639358 | 7.56 | 13 | | | | | | |
| chr17 | 6500095 | 6501554 | 7.20 | 11 | | | | | | |
| chr17 | 6866627 | 6868275 | 12.02 | 20 | | | | | + | 6867092 |
| chr17 | 6886114 | 6887363 | 7.45 | 14 | − | 6885672 | 6887966 | SLC16A11 | − | 6885672 |
| chr17 | 24967181 | 24969332 | 12.22 | 12 | | | | | − | 24965899 |
| chr17 | 27846829 | 27847493 | 7.17 | 13 | | | | | − | 27843740 |
| chr17 | 32371614 | 32374986 | 8.39 | 13 | + | 32368611 | 32374607 | LHX1 | + | 32368611 |
| chr17 | 34584727 | 34585356 | 9.69 | 18 | | | | | − | 34583234 |
| chr17 | 36937436 | 36938490 | 8.11 | 8 | | | | | | |
| chr17 | 38089183 | 38089828 | 5.65 | 9 | | | | | + | 38088157 |
| chr17 | 38189261 | 38190408 | 9.88 | 13 | | | | | + | 38186221 |
| chr17 | 41329931 | 41330876 | 7.71 | 22 | | | | | + | 41328944 |
| chr17 | 42251317 | 42252051 | 10.35 | 11 | | | | | | |
| chr17 | 44151655 | 44152294 | 8.85 | 14 | | | | | | |
| chr17 | 44158959 | 44159608 | 8.44 | 16 | | | | | − | 44157124 |
| chr17 | 44179727 | 44180381 | 9.17 | 17 | | | | | | |
| chr17 | 44564528 | 44565791 | 10.73 | 10 | | | | | | |
| chr17 | 45000264 | 45000898 | 7.21 | 11 | | | | | | |
| chr17 | 45396800 | 45398051 | 10.87 | 11 | | | | | | |
| chr17 | 55570838 | 55572341 | 9.24 | 18 | | | | | | |
| chr17 | 55853774 | 55854910 | 6.09 | 16 | | | | | | |
| chr17 | 56827847 | 56829192 | 7.61 | 8 | | | | | | |
| chr17 | 56829856 | 56830915 | 13.30 | 14 | | | | | | |
| chr17 | 69719958 | 69720487 | 9.44 | 11 | | | | | − | 69717735 |
| chr17 | 69781509 | 69782358 | 7.08 | 11 | | | | | | |
| chr17 | 76724071 | 76724700 | 6.36 | 8 | | | | | − | 76705690 |
| chr17 | 77420013 | 77420742 | 6.14 | 12 | | | | | − | 77418885 |
| chr17 | 77479685 | 77480419 | 7.24 | 13 | | | | | + | 77478995 |
| chr18 | 5186243 | 5187292 | 11.77 | 12 | | | | | − | 5133671 |
| chr18 | 26875494 | 26877443 | 12.24 | 16 | | | | | − | 26824049 |
| chr18 | 46048374 | 46049045 | 6.75 | 15 | − | 46049213 | 46062142 | MBD1 | | |
| chr18 | 53254565 | 53259819 | 10.42 | 18 | | | | | + | 53253914 |
| chr18 | 55091928 | 55092557 | 11.94 | 15 | | | | | | |
| chr18 | 72664418 | 72665346 | 7.60 | 10 | | | | | | |
| chr19 | 335010 | 335674 | 7.63 | 11 | | | | | | |
| chr19 | 554463 | 555102 | 6.81 | 10 | | | | | + | 540892 |
| chr19 | 1812475 | 1814434 | 8.50 | 13 | | | | | − | 1803398 |
| chr19 | 4279423 | 4279977 | 5.17 | 13 | | | | | − | 4275039 |
| chr19 | 9469724 | 9470804 | 10.30 | 11 | | | | | | |
| chr19 | 10258672 | 10259721 | 8.63 | 13 | + | 10258649 | 10260198 | ICAM4 | + | 10258649 |
| chr19 | 10558593 | 10559142 | 6.31 | 12 | | | | | | |
| chr19 | 11167307 | 11167656 | 5.37 | 19 | | | | | − | 11135943 |
| chr19 | 11420829 | 11421273 | 5.50 | 16 | | | | | + | 11407268 |
| chr19 | 13844524 | 13845198 | 8.03 | 18 | | | | | | |
| chr19 | 18389860 | 18391505 | 11.85 | 15 | | | | | | |
| chr19 | 40222452 | 40224104 | 9.89 | 21 | + | 40213373 | 40223193 | SCN1B | | |
| chr19 | 40407463 | 40408032 | 4.95 | 10 | − | 40407543 | 40411468 | FAM187B | | |
| chr19 | 40740718 | 40741547 | 8.85 | 11 | | | | | − | 40732934 |
| chr19 | 46008398 | 46009157 | 9.53 | 20 | | | | | | |
| chr19 | 46333149 | 46333903 | 9.66 | 20 | | | | | | |
| chr19 | 49455836 | 49456370 | 7.46 | 11 | | | | | | |
| chr19 | 50811149 | 50811873 | 7.00 | 18 | | | | | − | 50804497 |
| chr19 | 53516363 | 53517093 | 11.19 | 14 | | | | | | |
| chr19 | 53609703 | 53610162 | 5.98 | 8 | | | | | + | 53589943 |
| chr19 | 53891560 | 53892099 | 7.52 | 10 | | | | | + | 53891039 |
| chr19 | 54214396 | 54215050 | 5.80 | 11 | | | | | | |
| chr19 | 54327945 | 54328592 | 6.76 | 12 | | | | | + | 54314474 |
| chr19 | 54628533 | 54629188 | 7.58 | 16 | | | | | − | 54624467 |
| chr19 | 54631289 | 54632038 | 8.21 | 15 | | | | | − | 54624467 |
| chr19 | 56897971 | 56898600 | 8.40 | 22 | | | | | + | 56888404 |
| chr19 | 59157320 | 59157774 | 9.54 | 19 | | | | | | |
| chr19 | 60489952 | 60490616 | 6.44 | 19 | | | | | + | 60487345 |
| chr19 | 60721053 | 60721912 | 9.92 | 19 | + | 60691681 | 60722277 | LOC284297 | + | 60691681 |
| chr2 | 2752186 | 2752835 | 10.69 | 12 | | | | | | |
| chr2 | 10100088 | 10101856 | 10.60 | 16 | | | | | | |
| chr2 | 19416426 | 19417175 | 7.98 | 12 | | | | | − | 19414726 |
| chr2 | 19424458 | 19425187 | 8.49 | 17 | | | | | | |
| chr2 | 72226653 | 72228187 | 10.04 | 12 | | | | | − | 72209874 |
| chr2 | 74582914 | 74584780 | 8.71 | 8 | − | 74585677 | 74588329 | PCGF1 | + | 74583251 |
| chr2 | 86869500 | 86870049 | 8.28 | 11 | | | | | − | 86865239 |
| chr2 | 111591681 | 111594348 | 13.30 | 21 | + | 111206620 | 111592270 | ACOXL | | |
| chr2 | 115636321 | 115637260 | 13.24 | 12 | | | | | + | 115636201 |
| chr2 | 119322147 | 119323502 | 10.24 | 13 | | | | | | |
| chr2 | 119323782 | 119324421 | 11.62 | 12 | | | | | | |
| chr2 | 119630604 | 119631968 | 10.75 | 17 | − | 119630288 | 119632941 | C1QL2 | − | 119630288 |
| chr2 | 119905436 | 119906957 | 8.58 | 13 | | | | | + | 119905915 |
| chr2 | 128126805 | 128127459 | 5.72 | 8 | + | 128119908 | 128126682 | GPR17 | − | 128112466 |
| chr2 | 155263352 | 155264303 | 12.69 | 17 | | | | | | |
| chr2 | 156884504 | 156885470 | 9.52 | 21 | | | | | | |
| chr2 | 158161857 | 158162509 | 7.18 | 12 | | | | | − | 158091524 |
| chr2 | 160626827 | 160628085 | 6.25 | 14 | | | | | | |
| chr2 | 171276492 | 171277161 | 9.56 | 8 | − | 171277194 | 171279323 | LOC440925 | | |
| chr2 | 172659538 | 172660401 | 9.52 | 15 | | | | | + | 172658453 |
| chr2 | 172660882 | 172661546 | 8.49 | 19 | | | | | + | 172658453 |
| chr2 | 172673432 | 172675478 | 7.47 | 13 | | | | | − | 172672411 |
| chr2 | 176641194 | 176641858 | 10.10 | 15 | | | | | | |
| chr2 | 176653956 | 176656685 | 9.31 | 11 | − | 176653080 | 176656936 | EVX2 | − | 176653080 |
| chr2 | 176671999 | 176672948 | 7.88 | 16 | + | 176672775 | 176673734 | HOXD12 | + | 176672775 |
| chr2 | 176690151 | 176690900 | 10.31 | 17 | | | | | + | 176689737 |
| chr2 | 176737592 | 176738247 | 9.42 | 16 | | | | | + | 176737050 |
| chr2 | 200034962 | 200036119 | 8.63 | 15 | | | | | | |
| chr2 | 200041833 | 200043073 | 13.73 | 23 | | | | | + | 200041065 |
| chr2 | 219481739 | 219482468 | 8.63 | 13 | | | | | | |
| chr2 | 222869621 | 222872193 | 7.97 | 14 | | | | | + | 222871109 |
| chr2 | 222884739 | 222885493 | 8.59 | 17 | | | | | | |
| chr2 | 232103046 | 232104293 | 8.25 | 15 | | | | | | |
| chr2 | 232279406 | 232280745 | 13.71 | 9 | | | | | | |
| chr2 | 233442461 | 233443405 | 8.93 | 17 | | | | | | |
| chr2 | 236751568 | 236752820 | 7.95 | 11 | | | | | | |
| chr20 | 3167981 | 3168945 | 7.77 | 17 | | | | | | |
| chr20 | 21434032 | 21436910 | 6.89 | 8 | | | | | | |
| chr20 | 22505521 | 22506890 | 10.23 | 14 | | | | | − | 22489193 |
| chr20 | 22515240 | 22516065 | 7.90 | 14 | | | | | | |
| chr20 | 25076779 | 25077618 | 6.77 | 13 | | | | | | |
| chr20 | 36787544 | 36790818 | 19.61 | 16 | + | 36786518 | 36791429 | SLC32A1 | + | 36786518 |
| chr20 | 41251999 | 41252763 | 10.05 | 12 | | | | | | |
| chr20 | 41976531 | 41978482 | 10.08 | 20 | | | | | + | 41976905 |
| chr20 | 43469538 | 43470502 | 10.23 | 12 | | | | | + | 43468650 |
| chr20 | 43531029 | 43532764 | 7.57 | 11 | | | | | | |
| chr20 | 43885946 | 43886595 | 9.09 | 20 | − | 43885261 | 43889360 | TNNC2 | − | 43885261 |
| chr20 | 44072542 | 44073411 | 9.39 | 19 | | | | | + | 44070953 |
| chr20 | 44073717 | 44074646 | 12.54 | 22 | | | | | + | 44070953 |
| chr20 | 44119200 | 44121030 | 7.91 | 15 | | | | | + | 44091227 |
| chr21 | 21291294 | 21291968 | 10.35 | 10 | | | | | | |
| chr21 | 34962773 | 34964708 | 7.05 | 10 | | | | | + | 34963557 |
| chr21 | 36990087 | 36992825 | 11.85 | 15 | | | | | | |
| chr22 | 28039290 | 28041531 | 13.41 | 21 | − | 28038921 | 28041748 | RASL10A | − | 28038921 |
| chr22 | 36807805 | 36808832 | 5.62 | 12 | | | | | − | 36804089 |
| chr22 | 38114321 | 38115070 | 6.57 | 18 | | | | | | |
| chr3 | 2115949 | 2117089 | 11.08 | 19 | | | | | | |
| chr3 | 32254374 | 32255424 | 7.95 | 11 | | | | | | |
| chr3 | 50357760 | 50358604 | 8.66 | 9 | | | | | | |
| chr3 | 131176854 | 131177513 | 8.41 | 14 | | | | | + | 131175803 |
| chr3 | 138020672 | 138021936 | 18.36 | 10 | | | | | + | 138020550 |
| chr3 | 155629021 | 155629670 | 10.35 | 10 | | | | | − | 155538154 |
| chr3 | 159303324 | 159304476 | 14.83 | 12 | | | | | − | 159296493 |
| chr3 | 161649899 | 161651150 | 6.32 | 16 | | | | | | |
| chr3 | 173648073 | 173649732 | 9.01 | 18 | | | | | | |
| chr3 | 188870393 | 188870937 | 13.49 | 19 | | | | | | |
| chr4 | 560951 | 561800 | 9.51 | 11 | | | | | | |
| chr4 | 1397393 | 1398032 | 7.81 | 13 | | | | | | |
| chr4 | 2903227 | 2903861 | 7.18 | 10 | | | | | − | 2902086 |
| chr4 | 4905483 | 4906134 | 5.98 | 14 | | | | | | |
| chr4 | 5945893 | 5946532 | 7.96 | 15 | | | | | | |
| chr4 | 13134417 | 13135173 | 7.94 | 15 | | | | | | |
| chr4 | 13135429 | 13136158 | 6.92 | 13 | | | | | | |
| chr4 | 30332624 | 30333968 | 9.09 | 19 | | | | | + | 30331134 |
| chr4 | 41443929 | 41444657 | 8.23 | 16 | | | | | − | 41440855 |
| chr4 | 41570047 | 41570684 | 10.05 | 23 | | | | | | |
| chr4 | 41577137 | 41577766 | 10.06 | 15 | | | | | | |
| chr4 | 52612173 | 52613018 | 7.73 | 17 | | | | | | |
| chr4 | 54661877 | 54662826 | 14.11 | 17 | + | 54661004 | 54662879 | GSX2 | + | 54661004 |
| chr4 | 85623804 | 85624451 | 7.86 | 11 | | | | | | |
| chr4 | 85636686 | 85637940 | 7.21 | 15 | | | | | − | 85633459 |
| chr4 | 94974738 | 94975381 | 8.87 | 9 | | | | | | |
| chr4 | 111758660 | 111759824 | 11.15 | 19 | − | 111758028 | 111777957 | PITX2 | − | 111758028 |
| chr4 | 111761532 | 111764385 | 6.14 | 11 | | | | | − | 111758028 |
| chr4 | 122211474 | 122212323 | 18.69 | 9 | | | | | − | 122176231 |
| chr4 | 154932988 | 154933738 | 10.82 | 17 | | | | | | |
| chr4 | 174664270 | 174664919 | 9.49 | 14 | | | | | | |
| chr4 | 174674369 | 174675025 | 8.92 | 16 | | | | | | |
| chr4 | 174684839 | 174685488 | 6.03 | 12 | − | 174684226 | 174687953 | HAND2 | − | 174684226 |
| chr4 | 174688523 | 174689572 | 9.32 | 15 | | | | | + | 174688183 |
| chr5 | 2792454 | 2794206 | 7.35 | 10 | | | | | | |
| chr5 | 2805770 | 2806724 | 9.19 | 18 | | | | | + | 2805261 |
| chr5 | 3643658 | 3644997 | 8.79 | 15 | | | | | | |
| chr5 | 32747979 | 32750229 | 12.42 | 16 | | | | | + | 32747421 |
| chr5 | 37872516 | 37875378 | 15.39 | 13 | | | | | − | 37851509 |
| chr5 | 43027793 | 43028447 | 15.00 | 8 | | | | | | |
| chr5 | 43053743 | 43054382 | 16.74 | 9 | | | | | | |
| chr5 | 54554757 | 54555401 | 8.65 | 11 | | | | | | |
| chr5 | 63292326 | 63293075 | 10.85 | 20 | | | | | | |
| chr5 | 76151227 | 76151876 | 8.32 | 17 | | | | | + | 76150588 |
| chr5 | 76968098 | 76969322 | 7.73 | 9 | | | | | − | 76960292 |
| chr5 | 77176139 | 77176798 | 13.09 | 12 | | | | | | |
| chr5 | 140286488 | 140287347 | 15.62 | 17 | | | | | + | 140242037 |
| chr5 | 140742560 | 140743119 | 8.07 | 13 | | | | | + | 140733834 |
| chr5 | 140747458 | 140748016 | 8.51 | 17 | | | | | + | 140742650 |
| chr5 | 140767256 | 140768329 | 11.36 | 19 | | | | | + | 140762703 |
| chr5 | 145698688 | 145700318 | 10.46 | 12 | + | 145698779 | 145700276 | POU4F3 | + | 145698779 |
| chr5 | 154006904 | 154007543 | 11.15 | 12 | | | | | | |
| chr5 | 157030866 | 157031611 | 9.76 | 19 | | | | | | |
| chr5 | 158456493 | 158457132 | 12.40 | 25 | | | | | − | 158055500 |
| chr5 | 158463748 | 158464392 | 9.38 | 15 | | | | | | |
| chr5 | 158690162 | 158691536 | 7.47 | 12 | | | | | | |
| chr5 | 172596242 | 172597176 | 10.35 | 17 | | | | | | |
| chr5 | 172604911 | 172605563 | 6.26 | 13 | | | | | | |
| chr6 | 1550805 | 1554054 | 7.29 | 8 | | | | | | |
| chr6 | 5939707 | 5940356 | 6.99 | 18 | | | | | | |
| chr6 | 10497957 | 10499310 | 8.37 | 15 | | | | | | |
| chr6 | 10530143 | 10530792 | 6.97 | 11 | | | | | | |
| chr6 | 10994972 | 10995711 | 7.30 | 13 | | | | | | |
| chr6 | 26125798 | 26126758 | 9.25 | 13 | | | | | | |
| chr6 | 27215069 | 27215633 | 6.95 | 16 | + | 27215066 | 27215436 | HIST1H4I | − | 27214051 |
| chr6 | 27387778 | 27388647 | 13.86 | 15 | | | | | | |
| chr6 | 27948718 | 27949880 | 10.00 | 11 | − | 27948904 | 27949268 | HIST1H4L | − | 27948904 |
| chr6 | 28283085 | 28283729 | 13.91 | 13 | | | | | | |
| chr6 | 29063972 | 29064638 | 9.18 | 14 | | | | | | |
| chr6 | 30050074 | 30050923 | 5.05 | 13 | | | | | | |
| chr6 | 30818753 | 30820599 | 10.08 | 16 | − | 30818954 | 30820306 | IER3 | − | 30818954 |
| chr6 | 37611537 | 37612266 | 7.91 | 10 | | | | | | |
| chr6 | 39977327 | 39978066 | 7.40 | 15 | | | | | + | 39868770 |
| chr6 | 41447224 | 41447987 | 15.75 | 14 | | | | | | |
| chr6 | 41449448 | 41450517 | 15.53 | 10 | | | | | | |
| chr6 | 41623448 | 41624191 | 15.17 | 21 | | | | | + | 41622141 |
| chr6 | 42253599 | 42254248 | 12.27 | 14 | | | | | + | 42231121 |
| chr6 | 43720000 | 43721360 | 8.21 | 12 | | | | | | |
| chr6 | 78228956 | 78230415 | 10.96 | 17 | | | | | | |
| chr6 | 83130068 | 83131208 | 8.72 | 14 | | | | | | |
| chr6 | 85539298 | 85540860 | 9.21 | 12 | | | | | | |
| chr6 | 100173150 | 100173899 | 9.05 | 9 | | | | | | |
| chr6 | 101009995 | 101010637 | 15.23 | 15 | | | | | − | 100943470 |
| chr6 | 101011950 | 101013447 | 6.65 | 16 | | | | | − | 100943470 |
| chr6 | 112147783 | 112148610 | 9.95 | 13 | | | | | − | 112089177 |
| chr6 | 117192435 | 117193290 | 8.00 | 14 | | | | | − | 117180053 |
| chr6 | 137857127 | 137857980 | 13.22 | 14 | | | | | | |
| chr6 | 146797042 | 146797694 | 10.61 | 14 | | | | | + | 146390474 |
| chr6 | 150326335 | 150328184 | 32.01 | 19 | | | | | + | 150326835 |
| chr7 | 526958 | 527907 | 9.01 | 15 | | | | | | |
| chr7 | 5077779 | 5078727 | 6.16 | 15 | + | 5078263 | 5079380 | LOC389458 | + | 5078263 |
| chr7 | 8448505 | 8449259 | 14.36 | 21 | | | | | + | 8440109 |
| chr7 | 8449600 | 8450254 | 10.16 | 21 | | | | | + | 8440109 |
| chr7 | 25864688 | 25865353 | 11.40 | 14 | | | | | | |
| chr7 | 27093668 | 27094904 | 10.45 | 13 | | | | | | |
| chr7 | 27100570 | 27102300 | 8.47 | 12 | | | | | − | 27099138 |
| chr7 | 27156749 | 27161918 | 7.67 | 13 | − | 27159862 | 27162821 | HOXA7 | − | 27159862 |
| chr7 | 27177747 | 27179316 | 8.87 | 11 | | | | | − | 27176734 |
| chr7 | 27185469 | 27186510 | 12.70 | 12 | − | 27187300 | 27191360 | HOXA11 | | |
| chr7 | 27190648 | 27192605 | 14.02 | 16 | | | | | + | 27191551 |
| chr7 | 27193961 | 27196205 | 9.17 | 15 | + | 27191551 | 27195437 | HOXA11AS | + | 27191551 |
| chr7 | 27210155 | 27212509 | 8.41 | 11 | | | | | | |
| chr7 | 27215870 | 27217214 | 10.22 | 9 | | | | | | |
| chr7 | 27241669 | 27242703 | 6.76 | 12 | | | | | | |
| chr7 | 27248657 | 27253218 | 8.83 | 9 | + | 27248688 | 27252717 | EVX1 | + | 27248688 |
| chr7 | 27257469 | 27259401 | 8.39 | 19 | | | | | | |
| chr7 | 44119525 | 44120164 | 6.06 | 15 | − | 44120810 | 44129672 | POLD2 | + | 44110484 |
| chr7 | 44151320 | 44152244 | 7.95 | 18 | − | 44150394 | 44195547 | GCK | − | 44150394 |
| chr7 | 51352205 | 51352779 | 7.08 | 20 | | | | | | |
| chr7 | 63667353 | 63668297 | 6.38 | 12 | | | | | | |
| chr7 | 96488377 | 96489524 | 7.61 | 11 | − | 96487637 | 96492079 | DLX5 | − | 96487637 |
| chr7 | 101722805 | 101723452 | 5.94 | 15 | | | | | + | 101715165 |
| chr7 | 107429031 | 107429905 | 11.02 | 16 | | | | | − | 107351481 |
| chr7 | 116906858 | 116907422 | 10.33 | 12 | | | | | | |
| chr7 | 120755525 | 120756474 | 10.97 | 17 | | | | | + | 120752656 |
| chr7 | 127595227 | 127596086 | 10.92 | 12 | | | | | | |
| chr7 | 127697971 | 127698610 | 6.31 | 16 | | | | | | |
| chr7 | 129207840 | 129209778 | 8.11 | 15 | | | | | | |
| chr7 | 129210064 | 129210736 | 8.06 | 13 | | | | | | |
| chr7 | 129912506 | 129914030 | 8.21 | 18 | − | 129914133 | 129918249 | MESTIT1 | | |
| chr7 | 131983416 | 131984045 | 7.46 | 10 | | | | | | |
| chr7 | 148743033 | 148743764 | 17.18 | 22 | | | | | | |
| chr7 | 152252636 | 152253475 | 11.38 | 26 | | | | | | |
| chr7 | 154857518 | 154859486 | 8.64 | 14 | | | | | | |
| chr7 | 154951604 | 154952844 | 6.66 | 12 | | | | | | |
| chr7 | 155291555 | 155292209 | 7.84 | 17 | | | | | − | 155288318 |
| chr7 | 156125196 | 156126661 | 8.12 | 9 | | | | | | |
| chr7 | 158515940 | 158517089 | 7.67 | 11 | | | | | − | 158513626 |
| chr8 | 206253 | 206907 | 5.98 | 14 | | | | | | |
| chr8 | 10626015 | 10628284 | 7.94 | 10 | | | | | | |
| chr8 | 11574478 | 11576337 | 7.53 | 14 | | | | | | |
| chr8 | 23618422 | 23620876 | 8.71 | 14 | | | | | − | 23615909 |
| chr8 | 24826911 | 24828465 | 8.26 | 19 | | | | | + | 24827178 |
| chr8 | 24868882 | 24870630 | 9.36 | 13 | | | | | − | 24864387 |
| chr8 | 25965058 | 25965627 | 10.30 | 17 | | | | | | |
| chr8 | 26777575 | 26780521 | 15.03 | 22 | | | | | − | 26683138 |
| chr8 | 41543514 | 41544353 | 7.01 | 14 | | | | | | |
| chr8 | 49471563 | 49472223 | 8.17 | 8 | | | | | | |
| chr8 | 57522214 | 57523567 | 8.06 | 16 | | | | | | |
| chr8 | 65873955 | 65874718 | 9.27 | 15 | | | | | | |
| chr8 | 71144440 | 71147178 | 6.16 | 11 | | | | | − | 71126576 |
| chr8 | 95720330 | 95720979 | 6.32 | 19 | | | | | | |
| chr8 | 97238932 | 97239559 | 6.75 | 16 | | | | | − | 97223733 |
| chr8 | 98359200 | 98360130 | 6.68 | 12 | | | | | | |
| chr8 | 100029975 | 100030644 | 10.56 | 13 | | | | | + | 100025806 |
| chr8 | 102573379 | 102574213 | 9.31 | 19 | | | | | | |
| chr8 | 110725080 | 110726354 | 9.55 | 13 | | | | | − | 110655580 |
| chr8 | 145896215 | 145896964 | 8.22 | 19 | | | | | | |
| chr9 | 34369432 | 34371281 | 7.87 | 20 | − | 34369016 | 34387849 | C9orf24 | − | 34369016 |
| chr9 | 78818627 | 78820116 | 13.23 | 11 | | | | | | |
| chr9 | 85760867 | 85761816 | 9.92 | 8 | | | | | | |
| chr9 | 90339041 | 90340305 | 7.18 | 17 | | | | | | |
| chr9 | 99109616 | 99110142 | 9.01 | 14 | | | | | + | 99040599 |
| chr9 | 99304529 | 99305158 | 9.93 | 11 | | | | | | |
| chr9 | 124021380 | 124022605 | 12.53 | 13 | | | | | − | 124004678 |
| chr9 | 125810809 | 125811940 | 13.91 | 12 | | | | | | |
| chr9 | 129501281 | 129501822 | 7.67 | 12 | | | | | | |
| chr9 | 135557390 | 135558039 | 7.41 | 15 | | | | | − | 135518506 |
| chrX | 111211507 | 111212571 | 11.31 | 13 | | | | | − | 110904197 |
| chrY | 510532 | 512001 | 5.99 | 11 | | | | | + | 505078 |

| Peaks | | | | Gene | Upstream | | | Gene |
|---|---|---|---|---|---|---|---|---|
| chr | Start | End | End | Symbol | Strand | Start | End | Symbol |
| chr1 | 2456587 | 2457246 | | | | | | |
| chr1 | 37973297 | 37974046 | 38003411 | EPHA10 | | | | |
| chr1 | 38002438 | 38003587 | | | − | 37998531 | 38003411 | EPHA10 |
| chr1 | 46712589 | 46713323 | | | | | | |
| chr1 | 47469314 | 47470340 | | | | | | |
| chr1 | 54594420 | 54595152 | 54644680 | SSBP3 | | | | |
| chr1 | 60311689 | 60312524 | | | − | 60228653 | 60312014 | C1orf87 |
| chr1 | 63556597 | 63560345 | | | + | 63561317 | 63563385 | FOXD3 |
| chr1 | 90963290 | 90965440 | | | | | | |
| chr1 | 111614814 | 111615476 | | | | | | |
| chr1 | 119328183 | 119328832 | 119333702 | TBX15 | | | | |
| chr1 | 143012194 | 143012944 | 143053112 | LOC728875 | | | | |
| chr1 | 144674586 | 144675239 | | | | | | |
| chr1 | 144690585 | 144691329 | | | | | | |
| chr1 | 144702031 | 144702576 | | | | | | |
| chr1 | 145971587 | 145972240 | | | | | | |
| chr1 | 145987585 | 145988329 | | | | | | |
| chr1 | 145999042 | 145999587 | | | | | | |
| chr1 | 146184793 | 146185549 | | | | | | |
| chr1 | 146219303 | 146219957 | | | | | | |
| chr1 | 146248547 | 146249292 | | | | | | |
| chr1 | 146256160 | 146256719 | | | | | | |
| chr1 | 146466570 | 146467421 | | | | | | |
| chr1 | 147599481 | 147600124 | | | | | | |
| chr1 | 147600226 | 147600785 | | | | | | |
| chr1 | 147665315 | 147665969 | | | | | | |
| chr1 | 147882409 | 147883136 | | | | | | |
| chr1 | 147985011 | 147986318 | | | | | | |
| chr1 | 150078663 | 150079332 | 150083264 | LOC100132111 | − | 150076962 | 150079657 | LOC100191040 |
| chr1 | 150349668 | 150350912 | 150353180 | TCHH | | | | |
| chr1 | 153320294 | 153321238 | 153326638 | EFNA3 | | | | |
| chr1 | 153557083 | 153557827 | | | − | 153557263 | 153567533 | RUSC1 |
| chr1 | 154527610 | 154528254 | 154528858 | TMEM79 | | | | |
| chr1 | 155085184 | 155085851 | 155095290 | INSRR | | | | |
| chr1 | 165119820 | 165120544 | | | | | | |
| chr1 | 179719350 | 179719904 | | | + | 179719338 | 180037338 | CACNA1E |
| chr1 | 181421423 | 181422187 | | | + | 181421796 | 181480885 | LAMC2 |
| chr1 | 196156945 | 196157494 | 196165896 | LHX9 | | | | |
| chr1 | 207914587 | 207915953 | 207916358 | G0S2 | + | 207915292 | 207916358 | G0S2 |
| chr1 | 219131519 | 219132268 | | | | | | |
| chr1 | 219134095 | 219134834 | | | | | | |
| chr1 | 222002983 | 222003622 | 222030343 | CAPN2 | | | | |
| chr1 | 222870205 | 222872650 | 222994872 | CNIH3 | + | 222870801 | 222994872 | CNIH3 |
| chr1 | 226700008 | 226700767 | | | | | | |
| chr1 | 234752777 | 234754328 | 234779619 | LGALS8 | + | 234753699 | 234779619 | LGALS8 |
| chr10 | 7489395 | 7490153 | 7493456 | SFMBT2 | | | | |
| chr10 | 46412477 | 46413703 | | | + | 46413551 | 46420574 | GPRIN2 |
| chr10 | 50486546 | 50487915 | | | + | 50488352 | 50490772 | SLC18A3 |
| chr10 | 50488946 | 50491715 | 50490772 | SLC18A3 | + | 50492088 | 50543156 | CHAT |
| chr10 | 79066119 | 79066773 | 79067583 | KCNMA1 | − | 78314641 | 79067583 | KCNMA1 |
| chr10 | 81732969 | 81733640 | | | | | | |
| chr10 | 99070134 | 99071063 | 99071662 | FRAT1 | | | | |
| chr10 | 102580005 | 102580574 | | | | | | |
| chr10 | 102972304 | 102973663 | | | | | | |
| chr10 | 120344105 | 120345734 | 120345150 | PRLHR | − | 120342905 | 120345150 | PRLHR |
| chr10 | 124628733 | 124629607 | | | + | 124629138 | 124648220 | LOC399815 |
| chr11 | 1718992 | 1720660 | 1742077 | HCCA2 | | | | |
| chr11 | 1721203 | 1723357 | | | | | | |
| chr11 | 1727398 | 1728562 | | | | | | |
| chr11 | 2102906 | 2103345 | | | | | | |
| chr11 | 2183194 | 2184458 | | | | | | |
| chr11 | 10428000 | 10428654 | | | + | 10428799 | 10485702 | AMPD3 |
| chr11 | 14883948 | 14884302 | | | | | | |
| chr11 | 19220281 | 19221020 | | | | | | |
| chr11 | 22319354 | 22320095 | 22357620 | SLC17A6 | | | | |
| chr11 | 31789500 | 31795000 | 31789361 | PAX6 | − | 31794625 | 31865159 | DKFZp686K1684 |
| chr11 | 32411453 | 32412722 | 32413663 | WT1 | − | 32365900 | 32413663 | WT1 |
| chr11 | 44283814 | 44284478 | 44288292 | ALX4 | | | | |
| chr11 | 44935052 | 44935706 | | | | | | |
| chr11 | 63531706 | 63532455 | 63690161 | MACROD1 | | | | |
| chr11 | 63823533 | 63825290 | | | + | 63824438 | 63828815 | C11orf20 |
| chr11 | 64947186 | 64947820 | | | | | | |
| chr11 | 73049464 | 73050318 | 73051512 | PLEKHB1 | | | | |
| chr11 | 94523361 | 94524500 | | | | | | |
| chr11 | 124133793 | 124135432 | 124137433 | ESAM | | | | |
| chr11 | 133762389 | 133763138 | 133787022 | B3GAT1 | | | | |
| chr12 | 6803351 | 6804025 | 6806844 | GPR162 | | | | |
| chr12 | 21985422 | 21986671 | | | | | | |
| chr12 | 42231009 | 42232738 | | | − | 42034278 | 42231991 | ADAMTS20 |
| chr12 | 45511201 | 45511951 | | | | | | |
| chr12 | 47771091 | 47771619 | 47774869 | DHH | | | | |
| chr12 | 47975997 | 47977646 | 47978748 | PRPH | + | 47975175 | 47978748 | PRPH |
| chr12 | 52418168 | 52418832 | | | | | | |
| chr12 | 52646454 | 52647108 | 52648782 | HOTAIR | | | | |
| chr12 | 53049097 | 53049742 | | | | | | |
| chr12 | 55168271 | 55169242 | | | − | 55151002 | 55168448 | GLS2 |
| chr12 | 55918204 | 55919848 | 55920742 | NDUFA4L2 | − | 55914952 | 55920742 | NDUFA4L2 |
| chr12 | 56300983 | 56301939 | 56306201 | SLC26A10 | | | | |
| chr12 | 79626169 | 79626805 | 79627382 | MYF6 | + | 79625576 | 79627382 | MYF6 |
| chr12 | 84197229 | 84198765 | | | + | 84198166 | 84219692 | ALX1 |
| chr12 | 101868310 | 101868969 | | | | | | |
| chr12 | 113322635 | 113323304 | 113326086 | TBX5 | | | | |
| chr12 | 115800795 | 115801644 | 115803615 | HRK | | | | |
| chr13 | 27399628 | 27401487 | | | | | | |
| chr13 | 40928925 | 40929564 | | | + | 40929541 | 40943013 | C13orf15 |
| chr13 | 52211145 | 52212607 | | | − | 52175399 | 52211948 | LECT1 |
| chr13 | 102224101 | 102224965 | | | − | 102216465 | 102224150 | C13orf27 |
| chr13 | 112725390 | 112726034 | 112800863 | MCF2L | | | | |
| chr14 | 22360909 | 22361365 | | | | | | |
| chr14 | 23115638 | 23116287 | 23117849 | JPH4 | | | | |
| chr14 | 36061370 | 36062206 | | | | | | |
| chr14 | 36122209 | 36123483 | | | − | 36118966 | 36121537 | NKX2-8 |
| chr14 | 36194850 | 36195499 | | | | | | |
| chr14 | 36196539 | 36197988 | 36216762 | PAX9 | + | 36196532 | 36216762 | PAX9 |
| chr14 | 36201539 | 36202583 | 36216762 | PAX9 | | | | |
| chr14 | 37137508 | 37138866 | | | | | | |
| chr14 | 37748210 | 37750570 | 37752019 | SSTR1 | | | | |
| chr14 | 53488199 | 53488653 | 53493304 | BMP4 | | | | |
| chr14 | 53491636 | 53492707 | 53493304 | BMP4 | − | 53486204 | 53493304 | BMP4 |
| chr14 | 53499835 | 53500364 | | | | | | |
| chr14 | 56344682 | 56346630 | 56346937 | OTX2 | − | 56337177 | 56346937 | OTX2 |
| chr14 | 60021741 | 60022400 | | | − | 59972426 | 60022517 | C14orf39 |
| chr14 | 60043368 | 60044022 | | | | | | |
| chr14 | 60045099 | 60047942 | 60048278 | SIX6 | + | 60045690 | 60048278 | SIX6 |
| chr14 | 73776450 | 73777601 | 73799194 | VSX2 | + | 73775927 | 73799194 | VSX2 |
| chr14 | 74147083 | 74147922 | 74148787 | LTBP2 | − | 74034638 | 74148787 | LTBP2 |
| chr14 | 94304390 | 94305319 | 94306252 | GSC | − | 94304312 | 94306252 | GSC |
| chr14 | 94308930 | 94310289 | | | | | | |
| chr14 | 105023673 | 105024599 | | | + | 105024301 | 105026169 | CRIP1 |
| chr15 | 43195869 | 43196818 | 43197593 | DUOXA2 | | | | |
| chr15 | 43783619 | 43784273 | | | | | | |
| chr15 | 50867775 | 50868924 | 50869501 | ONECUT1 | − | 50836644 | 50869501 | ONECUT1 |
| chr15 | 50873886 | 50875041 | | | | | | |
| chr15 | 62984637 | 62985202 | | | | | | |
| chr15 | 65913980 | 65915431 | | | | | | |
| chr15 | 72207930 | 72209773 | 72216196 | ISLR2 | + | 72209795 | 72216196 | ISLR2 |
| chr15 | 74414585 | 74415136 | | | | | | |
| chr15 | 81112195 | 81112934 | 81113783 | CPEB1 | − | 81009005 | 81113783 | CPEB1 |
| chr15 | 82113943 | 82114802 | | | + | 82113841 | 82499597 | ADAMTSL3 |
| chr15 | 87755647 | 87756297 | | | | | | |
| chr16 | 62621 | 63380 | | | − | 48057 | 62629 | RHBDF1 |
| chr16 | 4977615 | 4978589 | 5009157 | SEC14L5 | | | | |
| chr16 | 19804086 | 19804635 | | | − | 19777793 | 19803652 | GPRC5B |
| chr16 | 29703730 | 29704362 | | | | | | |
| chr16 | 31014172 | 31014616 | | | − | 31009675 | 31013777 | VKORC1 |
| chr16 | 49432345 | 49433015 | | | | | | |
| chr16 | 49744810 | 49746252 | | | | | | |
| chr16 | 52873128 | 52875454 | | | | | | |
| chr16 | 52879121 | 52880054 | | | | | | |
| chr16 | 53523342 | 53524273 | 53525896 | IRX5 | + | 53522611 | 53525896 | IRX5 |
| chr16 | 53527819 | 53530073 | | | | | | |
| chr16 | 55229290 | 55229739 | | | + | 55230078 | 55231500 | MT1A |
| chr16 | 55234406 | 55235250 | | | + | 55235216 | 55236353 | MT1DP |
| chr16 | 55254245 | 55254911 | | | | | | |
| chr16 | 56575924 | 56577738 | 56579518 | TEPP | | | | |
| chr16 | 65747571 | 65748520 | 65751313 | TRADD | | | | |
| chr17 | 1901278 | 1904018 | | | | | | |
| chr17 | 4385916 | 4386650 | 4389977 | SPNS2 | | | | |
| chr17 | 4635389 | 4636518 | 4636469 | VMO1 | − | 4635320 | 4636469 | VMO1 |
| chr17 | 4638694 | 4639358 | | | + | 4638993 | 4640425 | GLTPD2 |
| chr17 | 6500095 | 6501554 | | | | | | |
| chr17 | 6866627 | 6868275 | 6873685 | BCL6B | + | 6867092 | 6873685 | BCL6B |
| chr17 | 6886114 | 6887363 | 6887966 | SLC16A11 | − | 6885672 | 6887966 | SLC16A11 |
| chr17 | 24967181 | 24969332 | 24972620 | CORO6 | | | | |
| chr17 | 27846829 | 27847493 | 28228015 | MYO1D | | | | |
| chr17 | 32371614 | 32374986 | 32374607 | LHX1 | | | | |
| chr17 | 34584727 | 34585356 | 34607427 | CACNB1 | | | | |
| chr17 | 36937436 | 36938490 | | | − | 36933394 | 36938167 | KRT19 |
| chr17 | 38089183 | 38089828 | 38105536 | CNTNAP1 | | | | |
| chr17 | 38189261 | 38190408 | 38202610 | WNK4 | | | | |
| chr17 | 41329931 | 41330876 | 41331959 | LOC100130148 | + | 41328944 | 41331959 | LOC100130148 |
| chr17 | 42251317 | 42252051 | | | − | 42196856 | 42251081 | WNT3 |
| chr17 | 44151655 | 44152294 | | | | | | |
| chr17 | 44158959 | 44159608 | 44161110 | HOXB13 | | | | |
| chr17 | 44179727 | 44180381 | | | | | | |
| chr17 | 44564528 | 44565791 | | | + | 44565327 | 44602350 | B4GALNT2 |
| chr17 | 45000264 | 45000898 | | | | | | |
| chr17 | 45396800 | 45398051 | | | | | | |
| chr17 | 55570838 | 55572341 | | | | | | |
| chr17 | 55853774 | 55854910 | | | + | 55854646 | 55863568 | C17orf64 |
| chr17 | 56827847 | 56829192 | | | | | | |
| chr17 | 56829856 | 56830915 | | | | | | |
| chr17 | 69719958 | 69720487 | 69721055 | MGC16275 | + | 69721290 | 69769752 | TTYH2 |
| chr17 | 69781509 | 69782358 | | | + | 69781991 | 69822617 | DNAI2 |
| chr17 | 76724071 | 76724700 | 76754467 | AATK | | | | |
| chr17 | 77420013 | 77420742 | 77422527 | ARHGDIA | | | | |
| chr17 | 77479685 | 77480419 | 77481919 | LOC92659 | + | 77478995 | 77481919 | LOC92659 |
| chr18 | 5186243 | 5187292 | 5187255 | LOC642597 | − | 5133671 | 5187255 | LOC642597 |
| chr18 | 26875494 | 26877443 | 26876779 | DSC3 | − | 26824049 | 26876779 | DSC3 |
| chr18 | 46048374 | 46049045 | | | | | | |
| chr18 | 53254565 | 53259819 | 53309528 | ONECUT2 | + | 53253914 | 53309528 | ONECUT2 |
| chr18 | 55091928 | 55092557 | | | − | 55085246 | 55091605 | RAX |
| chr18 | 72664418 | 72665346 | | | + | 72665103 | 72811670 | ZNF236 |
| chr19 | 335010 | 335674 | | | | | | |
| chr19 | 554463 | 555102 | 568159 | HCN2 | | | | |
| chr19 | 1812475 | 1814434 | 1814564 | KLF16 | − | 1803398 | 1814564 | KLF16 |
| chr19 | 4279423 | 4279977 | 4289847 | STAP2 | | | | |
| chr19 | 9469724 | 9470804 | | | − | 9438030 | 9470279 | ZNF560 |
| chr19 | 10258672 | 10259721 | 10260198 | ICAM4 | + | 10258649 | 10260198 | ICAM4 |
| chr19 | 10558593 | 10559142 | | | − | 10544347 | 10558991 | AP1M2 |
| chr19 | 11167307 | 11167656 | 11169224 | KANK2 | | | | |
| chr19 | 11420829 | 11421273 | 11422782 | PRKCSH | | | | |
| chr19 | 13844524 | 13845198 | | | | | | |
| chr19 | 18389860 | 18391505 | | | + | 18391135 | 18406459 | SSBP4 |
| chr19 | 40222452 | 40224104 | | | + | 40223249 | 40249315 | HPN |
| chr19 | 40407463 | 40408032 | | | | | | |
| chr19 | 40740718 | 40741547 | 40746400 | ATP4A | | | | |
| chr19 | 46008398 | 46009157 | | | | | | |
| chr19 | 46333149 | 46333903 | | | | | | |
| chr19 | 49455836 | 49456370 | | | + | 49455915 | 49471308 | ZNF233 |
| chr19 | 50811149 | 50811873 | 50834509 | EML2 | | | | |
| chr19 | 53516363 | 53517093 | | | | | | |
| chr19 | 53609703 | 53610162 | 53640000 | GRIN2D | | | | |
| chr19 | 53891560 | 53892099 | 53901003 | FUT2 | + | 53891039 | 53901003 | FUT2 |
| chr19 | 54214396 | 54215050 | | | | | | |
| chr19 | 54327945 | 54328592 | 54346092 | PPFIA3 | | | | |
| chr19 | 54628533 | 54629188 | 54636620 | SLC17A7 | | | | |
| chr19 | 54631289 | 54632038 | 54636620 | SLC17A7 | | | | |
| chr19 | 56897971 | 56898600 | 56900254 | NCRNA00085 | | | | |
| chr19 | 59157320 | 59157774 | | | + | 59158105 | 59177951 | CACNG8 |
| chr19 | 60489952 | 60490616 | 60515715 | BRSK1 | | | | |
| chr19 | 60721053 | 60721912 | 60722277 | LOC284297 | | | | |
| chr2 | 2752186 | 2752835 | | | | | | |
| chr2 | 10100088 | 10101856 | | | + | 10101132 | 10112414 | KLF11 |
| chr2 | 19416426 | 19417175 | 19421853 | OSR1 | | | | |
| chr2 | 19424458 | 19425187 | | | | | | |
| chr2 | 72226653 | 72228187 | 72228471 | CYP26B1 | − | 72209874 | 72228471 | CYP26B1 |
| chr2 | 74582914 | 74584780 | 74585700 | LOC151534 | + | 74583251 | 74585700 | LOC151534 |
| chr2 | 86869500 | 86870049 | 86889030 | CD8A | | | | |
| chr2 | 111591681 | 111594348 | | | + | 111594961 | 111642493 | BCL2L11 |
| chr2 | 115636321 | 115637260 | 116318406 | DPP10 | + | 115636201 | 116318406 | DPP10 |
| chr2 | 119322147 | 119323502 | | | − | 119316216 | 119322229 | EN1 |
| chr2 | 119323782 | 119324421 | | | | | | |
| chr2 | 119630604 | 119631968 | 119632941 | C1QL2 | − | 119630288 | 119632941 | C1QL2 |
| chr2 | 119905436 | 119906957 | 119912566 | TMEM37 | + | 119905915 | 119912566 | TMEM37 |
| chr2 | 128126805 | 128127459 | 128155830 | LIMS2 | | | | |
| chr2 | 155263352 | 155264303 | | | + | 155263338 | 155421260 | KCNJ3 |
| chr2 | 156884504 | 156885470 | | | | | | |
| chr2 | 158161857 | 158162509 | 158193645 | ACVR1C | − | 158091524 | 158162318 | ACVR1C |
| chr2 | 160626827 | 160628085 | | | − | 160510571 | 160627367 | PLA2R1 |
| chr2 | 171276492 | 171277161 | | | | | | |
| chr2 | 172659538 | 172660401 | 172662647 | DLX1 | | | | |
| chr2 | 172660882 | 172661546 | 172662647 | DLX1 | | | | |
| chr2 | 172673432 | 172675478 | 172675724 | DLX2 | − | 172672411 | 172675724 | DLX2 |
| chr2 | 176641194 | 176641858 | | | | | | |
| chr2 | 176653956 | 176656685 | 176656936 | EVX2 | − | 176653080 | 176656936 | EVX2 |
| chr2 | 176671999 | 176672948 | 176673734 | HOXD12 | + | 176672775 | 176673734 | HOXD12 |
| chr2 | 176690151 | 176690900 | 176692916 | HOXD10 | + | 176689737 | 176692916 | HOXD10 |
| chr2 | 176737592 | 176738247 | 176746072 | HOXD3 | + | 176737050 | 176746072 | HOXD3 |
| chr2 | 200034962 | 200036119 | | | | | | |
| chr2 | 200041833 | 200043073 | 200045724 | FLJ32063 | + | 200041065 | 200045724 | FLJ32063 |
| chr2 | 219481739 | 219482468 | | | | | | |
| chr2 | 222869621 | 222872193 | 222878180 | CCDC140 | + | 222871109 | 222878180 | CCDC140 |
| chr2 | 222884739 | 222885493 | | | | | | |
| chr2 | 232103046 | 232104293 | | | − | 232096114 | 232103426 | NMUR1 |
| chr2 | 232279406 | 232280745 | | | + | 232281478 | 232286494 | PTMA |
| chr2 | 233442461 | 233443405 | | | + | 233443237 | 233449351 | UNQ830 |
| chr2 | 236751568 | 236752820 | | | | | | |
| chr20 | 3167981 | 3168945 | | | | | | |
| chr20 | 21434032 | 21436910 | | | | | | |
| chr20 | 22505521 | 22506890 | 22507280 | C20orf56 | − | 22489193 | 22507280 | C20orf56 |
| chr20 | 22515240 | 22516065 | | | | | | |
| chr20 | 25076779 | 25077618 | | | − | 25069433 | 25077426 | LOC284798 |
| chr20 | 36787544 | 36790818 | 36791429 | SLC32A1 | | | | |
| chr20 | 41251999 | 41252763 | | | − | 40134805 | 41251971 | PTPRT |
| chr20 | 41976531 | 41978482 | 42131668 | TOX2 | + | 41978195 | 42131668 | TOX2 |
| chr20 | 43469538 | 43470502 | 43472661 | DBNDD2 | + | 43470328 | 43472661 | DBNDD2 |
| chr20 | 43531029 | 43532764 | | | + | 43531807 | 43543586 | WFDC2 |
| chr20 | 43885946 | 43886595 | 43889360 | TNNC2 | | | | |
| chr20 | 44072542 | 44073411 | 44078607 | MMP9 | | | | |
| chr20 | 44073717 | 44074646 | 44078607 | MMP9 | | | | |
| chr20 | 44119200 | 44121030 | 44122196 | SLC12A5 | | | | |
| chr21 | 21291294 | 21291968 | | | + | 21292503 | 21833085 | NCAM2 |
| chr21 | 34962773 | 34964708 | 35012389 | CLIC6 | + | 34963557 | 35012389 | CLIC6 |
| chr21 | 36990087 | 36992825 | | | | | | |
| chr22 | 28039290 | 28041531 | 28041748 | RASL10A | − | 28038921 | 28041748 | RASL10A |
| chr22 | 36807805 | 36808832 | 36809116 | SLC16A8 | − | 36804089 | 36809116 | SLC16A8 |
| chr22 | 38114321 | 38115070 | | | | | | |
| chr3 | 2115949 | 2117089 | | | + | 2117246 | 3074645 | CNTN4 |
| chr3 | 32254374 | 32255424 | | | + | 32255174 | 32386817 | CMTM8 |
| chr3 | 50357760 | 50358604 | | | − | 50353540 | 50358160 | ZMYND10 |
| chr3 | 131176854 | 131177513 | 131179466 | TRH | | | | |
| chr3 | 138020672 | 138021936 | 138057424 | TMEM22 | + | 138020550 | 138057424 | TMEM22 |
| chr3 | 155629021 | 155629670 | 155630198 | GPR149 | − | 155538154 | 155630198 | GPR149 |
| chr3 | 159303324 | 159304476 | 159306630 | SHOX2 | − | 159296493 | 159303520 | SHOX2 |
| chr3 | 161649899 | 161651150 | | | − | 161635984 | 161650320 | TRIM59 |
| chr3 | 173648073 | 173649732 | | | − | 173648027 | 173648897 | GHSR |
| chr3 | 188870393 | 188870937 | | | − | 188869387 | 188870895 | SST |
| chr4 | 560951 | 561800 | | | | | | |
| chr4 | 1397393 | 1398032 | | | | | | |
| chr4 | 2903227 | 2903861 | 2906384 | MFSD10 | | | | |
| chr4 | 4905483 | 4906134 | | | | | | |
| chr4 | 5945893 | 5946532 | | | − | 5873391 | 5945686 | CRMP1 |
| chr4 | 13134417 | 13135173 | | | | | | |
| chr4 | 13135429 | 13136158 | | | | | | |
| chr4 | 30332624 | 30333968 | 30757519 | PCDH7 | | | | |
| chr4 | 41443929 | 41444657 | 41445744 | PHOX2B | | | | |
| chr4 | 41570047 | 41570684 | | | | | | |
| chr4 | 41577137 | 41577766 | | | | | | |
| chr4 | 52612173 | 52613018 | | | + | 52612349 | 52658215 | SPATA18 |
| chr4 | 54661877 | 54662826 | 54662879 | GSX2 | + | 54661004 | 54662879 | GSX2 |
| chr4 | 85623804 | 85624451 | | | | | | |
| chr4 | 85636686 | 85637940 | 85638411 | NKX6-1 | − | 85633459 | 85638411 | NKX6-1 |
| chr4 | 94974738 | 94975381 | | | | | | |
| chr4 | 111758660 | 111759824 | 111777957 | PITX2 | | | | |
| chr4 | 111761532 | 111764385 | 111777957 | PITX2 | | | | |
| chr4 | 122211474 | 122212323 | 122213123 | C4orf31 | − | 122176231 | 122213123 | C4orf31 |
| chr4 | 154932988 | 154933738 | | | | | | |
| chr4 | 174664270 | 174664919 | | | | | | |
| chr4 | 174674369 | 174675025 | | | | | | |
| chr4 | 174684839 | 174685488 | 174687953 | HAND2 | | | | |
| chr4 | 174688523 | 174689572 | 174699556 | NBLA00301 | + | 174688183 | 174699556 | NBLA00301 |
| chr5 | 2792454 | 2794206 | | | | | | |
| chr5 | 2805770 | 2806724 | 2808511 | C5orf38 | + | 2805261 | 2808511 | C5orf38 |
| chr5 | 3643658 | 3644997 | | | | | | |
| chr5 | 32747979 | 32750229 | 32823011 | NPR3 | + | 32747421 | 32823011 | NPR3 |
| chr5 | 37872516 | 37875378 | 37875539 | GDNF | − | 37851509 | 37875539 | GDNF |
| chr5 | 43027793 | 43028447 | | | | | | |
| chr5 | 43053743 | 43054382 | | | | | | |
| chr5 | 54554757 | 54555401 | | | | | | |
| chr5 | 63292326 | 63293075 | | | − | 63292033 | 63293302 | HTR1A |
| chr5 | 76151227 | 76151876 | 76166895 | F2RL1 | + | 76150588 | 76166895 | F2RL1 |
| chr5 | 76968098 | 76969322 | 76970278 | OTP | − | 76960292 | 76970278 | OTP |
| chr5 | 77176139 | 77176798 | | | | | | |
| chr5 | 140286488 | 140287347 | 140372113 | PCDHA13 | + | 140286485 | 140372113 | PCDHAC1 |
| chr5 | 140742560 | 140743119 | 140872730 | PCDHGA6 | + | 140742650 | 140872730 | PCDHGA7 |
| chr5 | 140747458 | 140748016 | 140872730 | PCDHGA7 | + | 140747635 | 140872730 | PCDHGB4 |
| chr5 | 140767256 | 140768329 | 140872730 | PCDHGA9 | + | 140767953 | 140872730 | PCDHGB6 |
| chr5 | 145698688 | 145700318 | 145700276 | POU4F3 | + | 145698779 | 145700276 | POU4F3 |
| chr5 | 154006904 | 154007543 | | | | | | |
| chr5 | 157030866 | 157031611 | | | + | 157031138 | 157039738 | C5orf52 |
| chr5 | 158456493 | 158457132 | 158459366 | EBF1 | | | | |
| chr5 | 158463748 | 158464392 | | | | | | |
| chr5 | 158690162 | 158691536 | | | − | 158674368 | 158690059 | IL12B |
| chr5 | 172596242 | 172597176 | | | | | | |
| chr5 | 172604911 | 172605563 | | | | | | |
| chr6 | 1550805 | 1554054 | | | | | | |
| chr6 | 5939707 | 5940356 | | | | | | |
| chr6 | 10497957 | 10499310 | | | | | | |
| chr6 | 10530143 | 10530792 | | | | | | |
| chr6 | 10994972 | 10995711 | | | + | 10995049 | 11082527 | SYCP2L |
| chr6 | 26125798 | 26126758 | | | − | 26125238 | 26126019 | HIST1H1A |
| chr6 | 27215069 | 27215633 | 27222598 | HIST1H2BK | | | | |
| chr6 | 27387778 | 27388647 | | | | | | |
| chr6 | 27948718 | 27949880 | 27949268 | HIST1H4L | − | 27948904 | 27949268 | HIST1H4L |
| chr6 | 28283085 | 28283729 | | | | | | |
| chr6 | 29063972 | 29064638 | | | | | | |
| chr6 | 30050074 | 30050923 | | | + | 30050870 | 30054156 | HCG9 |
| chr6 | 30818753 | 30820599 | 30820306 | IER3 | − | 30818954 | 30820306 | IER3 |
| chr6 | 37611537 | 37612266 | | | | | | |
| chr6 | 39977327 | 39978066 | 39980619 | DAAM2 | | | | |
| chr6 | 41447224 | 41447987 | | | | | | |
| chr6 | 41449448 | 41450517 | | | | | | |
| chr6 | 41623448 | 41624191 | 41678100 | FOXP4 | | | | |
| chr6 | 42253599 | 42254248 | 42255770 | GUCA1A | | | | |
| chr6 | 43720000 | 43721360 | | | + | 43720787 | 43746722 | RSPH9 |
| chr6 | 78228956 | 78230415 | | | − | 78228666 | 78229839 | HTR1B |
| chr6 | 83130068 | 83131208 | | | + | 83130257 | 83133335 | TPBG |
| chr6 | 85539298 | 85540860 | | | | | | |
| chr6 | 100173150 | 100173899 | | | | | | |
| chr6 | 101009995 | 101010637 | 101018272 | SIM1 | | | | |
| chr6 | 101011950 | 101013447 | 101018272 | SIM1 | | | | |
| chr6 | 112147783 | 112148610 | 112301320 | FYN | − | 112089177 | 112147958 | FYN |
| chr6 | 117192435 | 117193290 | 117193579 | FAM162B | − | 117180053 | 117193579 | FAM162B |
| chr6 | 137857127 | 137857980 | | | − | 137855028 | 137857224 | OLIG3 |
| chr6 | 146797042 | 146797694 | 146800424 | GRM1 | | | | |
| chr6 | 150326335 | 150328184 | 150336539 | ULBP1 | + | 150326835 | 150336539 | ULBP1 |
| chr7 | 526958 | 527907 | | | − | 503424 | 526007 | PDGFA |
| chr7 | 5077779 | 5078727 | 5079380 | LOC389458 | + | 5078263 | 5079380 | LOC389458 |
| chr7 | 8448505 | 8449259 | 8759118 | NXPH1 | | | | |
| chr7 | 8449600 | 8450254 | 8759118 | NXPH1 | | | | |
| chr7 | 25864688 | 25865353 | | | | | | |
| chr7 | 27093668 | 27094904 | | | | | | |
| chr7 | 27100570 | 27102300 | 27102150 | HOXA1 | − | 27099138 | 27102150 | HOXA1 |
| chr7 | 27156749 | 27161918 | 27162821 | HOXA7 | − | 27159862 | 27162821 | HOXA7 |
| chr7 | 27177747 | 27179316 | 27186401 | HOXA10 | | | | |
| chr7 | 27185469 | 27186510 | | | − | 27176734 | 27186401 | HOXA10 |
| chr7 | 27190648 | 27192605 | 27195437 | HOXA11AS | + | 27191551 | 27195437 | HOXA11AS |
| chr7 | 27193961 | 27196205 | 27195437 | HOXA11AS | | | | |
| chr7 | 27210155 | 27212509 | | | | | | |
| chr7 | 27215870 | 27217214 | | | | | | |
| chr7 | 27241669 | 27242703 | | | | | | |
| chr7 | 27248657 | 27253218 | 27252717 | EVX1 | + | 27248688 | 27252717 | EVX1 |
| chr7 | 27257469 | 27259401 | | | | | | |
| chr7 | 44119525 | 44120164 | 44120684 | AEBP1 | | | | |
| chr7 | 44151320 | 44152244 | 44195547 | GCK | | | | |
| chr7 | 51352205 | 51352779 | | | − | 51051402 | 51352009 | COBL |
| chr7 | 63667353 | 63668297 | | | | | | |
| chr7 | 96488377 | 96489524 | 96492079 | DLX5 | | | | |
| chr7 | 101722805 | 101723452 | 101748898 | SH2B2 | | | | |
| chr7 | 107429031 | 107429905 | 107431040 | LAMB1 | | | | |
| chr7 | 116906858 | 116907422 | | | + | 116907252 | 117095954 | CFTR |
| chr7 | 120755525 | 120756474 | 120768394 | WNT16 | + | 120756325 | 120768394 | WNT16 |
| chr7 | 127595227 | 127596086 | | | | | | |
| chr7 | 127697971 | 127698610 | | | | | | |
| chr7 | 129207840 | 129209778 | | | | | | |
| chr7 | 129210064 | 129210736 | | | | | | |
| chr7 | 129912506 | 129914030 | | | + | 129913281 | 129933367 | MEST |
| chr7 | 131983416 | 131984045 | | | − | 131820060 | 131983987 | PLXNA4 |
| chr7 | 148743033 | 148743764 | | | | | | |
| chr7 | 152252636 | 152253475 | | | | | | |
| chr7 | 154857518 | 154859486 | | | | | | |
| chr7 | 154951604 | 154952844 | | | | | | |
| chr7 | 155291555 | 155292209 | 155297728 | SHH | | | | |
| chr7 | 156125196 | 156126661 | | | + | 156126161 | 156162581 | RNF32 |
| chr7 | 158515940 | 158517089 | 158630410 | VIPR2 | | | | |
| chr8 | 206253 | 206907 | | | | | | |
| chr8 | 10626015 | 10628284 | | | − | 10618687 | 10625432 | SOX7 |
| chr8 | 11574478 | 11576337 | | | | | | |
| chr8 | 23618422 | 23620876 | 23619867 | NKX2-6 | − | 23615909 | 23619867 | NKX2-6 |
| chr8 | 24826911 | 24828465 | 24832511 | NEFM | + | 24828359 | 24832511 | NEFM |
| chr8 | 24868882 | 24870630 | 24870048 | NEFL | − | 24864387 | 24870048 | NEFL |
| chr8 | 25965058 | 25965627 | | | | | | |
| chr8 | 26777575 | 26780521 | 26778839 | ADRA1A | − | 26683138 | 26778839 | ADRA1A |
| chr8 | 41543514 | 41544353 | | | | | | |
| chr8 | 49471563 | 49472223 | | | | | | |
| chr8 | 57522214 | 57523567 | | | − | 57516068 | 57521836 | PENK |
| chr8 | 65873955 | 65874718 | | | − | 65671082 | 65873902 | CYP7B1 |
| chr8 | 71144440 | 71147178 | 71146116 | PRDM14 | − | 71126576 | 71146116 | PRDM14 |
| chr8 | 95720330 | 95720979 | | | | | | |
| chr8 | 97238932 | 97239559 | 97242196 | GDF6 | | | | |
| chr8 | 98359200 | 98360130 | | | − | 98354889 | 98359352 | TSPYL5 |
| chr8 | 100029975 | 100030644 | 100033500 | OSR2 | | | | |
| chr8 | 102573379 | 102574213 | | | + | 102573843 | 102751128 | GRHL2 |
| chr8 | 110725080 | 110726354 | 110773196 | GOLSYN | − | 110655580 | 110726998 | GOLSYN |
| chr8 | 145896215 | 145896964 | | | | | | |
| chr9 | 34369432 | 34371281 | 34387849 | C9orf24 | − | 34369016 | 34371598 | C9orf24 |
| chr9 | 78818627 | 78820116 | | | | | | |
| chr9 | 85760867 | 85761816 | | | − | 85743046 | 85761483 | C9orf64 |
| chr9 | 90339041 | 90340305 | | | + | 90339835 | 90380523 | NXNL2 |
| chr9 | 99109616 | 99110142 | 99179390 | KIAA1529 | | | | |
| chr9 | 99304529 | 99305158 | | | | | | |
| chr9 | 124021380 | 124022605 | 124030840 | LHX6 | | | | |
| chr9 | 125810809 | 125811940 | | | | | | |
| chr9 | 129501281 | 129501822 | | | | | | |
| chr9 | 135557390 | 135558039 | 135594898 | SARDH | | | | |
| chrX | 111211507 | 111212571 | 111212660 | TRPC5 | + | 111212908 | 111587129 | ZCCHC16 |
| chrY | 510532 | 512001 | 540146 | SHOX | | | | |

* This column shows the Average P value score of the peak in all tumors, excluding the tumors that do not have this peak.

TABLE 2
Frequently methylated regions in grade I tumors

| | | | | No. of grade I | |
|---|---|---|---|---|---|
| Seq. ID | | | | tumors | Adjacent |
| NO. | Chr | Start | End | methylated | gene |
| 2 | chr5 | 158456493 | 158457132 | 6 | EBF1 |
| 11 | chr1 | 2456587 | 2457246 | 5 | |
| 12 | chr1 | 147665315 | 147665969 | 5 | |
| 13 | chr11 | 1718992 | 1720660 | 5 | HCCA2 |
| 14 | chr11 | 44935052 | 44935706 | 5 | |
| 15 | chr17 | 4385916 | 4386650 | 5 | |
| 16 | chr19 | 554463 | 555102 | 5 | HCN2 |
| 17 | chr7 | 44119525 | 44120164 | 5 | POLD2 |

TABLE 3
Functional annotation of 5′-end methylated genes in tumor samples

| | | | P | | |
|---|---|---|---|---|---|
| GO ID | GO Term | Count | Value | Benjamini | Genes |
| GO: 0048513 | organ | 34 | 7.50E−09 | 1.33E−05 | IL12B, PDGFA, LECT1, BMP4, OTX2, |
| | development | | | | MYF6, CNTN4, PAX9, FYN, SHOX2, |
| | | | | | EN1, CHAT, GSX2, KRT19, SIX6, |
| | | | | | POU4F3, RAX, FOXD3, GDNF, |
| | | | | | HOXD12, NPR3, HOXD3, HOXD10, |
| | | | | | MEST, ONECUT2, ALX1, NKX2-6, |
| | | | | | ONECUT1, OTP, GHSR, LAMC2, DLX2, |
| | | | | | NKX6-1, CFTR |
| GO: 0007420 | brain | 11 | 1.31E−06 | 1.16E−03 | CNTN4, ALX1, RAX, OTP, FYN, EN1, |
| | development | | | | DLX2, BMP4, GSX2, OTX2, CFTR |
| GO: 0007399 | nervous system | 22 | 3.38E−06 | 1.99E−03 | POU4F3, RAX, NCAM2, GDNF, BMP4, |
| | development | | | | OTX2, NEFM, PCDHAC1, CNTN4, ALX1, |
| | | | | | OTP, FYN, GHSR, EN1, SHOX2, DLX2, |
| | | | | | CHAT, NKX6-1, GSX2, CRMP1, NKX2-8, |
| | | | | | CFTR |
| GO: 0031323 | regulation of | 47 | 8.01E−06 | 3.54E−03 | ZNF560, ZNF233, EVX2, IL12B, PDGFA, |
| | cellular | | | | BMP4, VSX2, OTX2, KLF11, MYF6, |
| | metabolic | | | | IRX5, PAX9, GSC, KLF16, EN1, SHOX2, |
| | process | | | | GSX2, GRHL2, SOX7, SIX6, NKX2-8, |
| | | | | | CPEB1, TOX2, POU4F3, RAX, FOXD3, |
| | | | | | BCL6B, HOXD12, HOXD3, HOXD10, |
| | | | | | ZNF236, ONECUT2, ALX1, C14orf39, |
| | | | | | EVX1, HOXA1, ONECUT1, NKX2-6, |
| | | | | | OLIG3, OTP, PRDM14, GHSR, DLX2, |
| | | | | | NKX6-1, HOXA10, WT1, HOXA7 |
| GO: 0022008 | neurogenesis | 13 | 1.49E−05 | 1.28E−02 | POU4F3, GDNF, OTX2, NEFM, CNTN4, |
| | | | | | OTP, FYN, EN1, CHAT, GSX2, NKX6-1, |
| | | | | | NKX2-8, CFTR |
| GO: 0048699 | generation of | 12 | 4.40E−05 | 1.29E−02 | CNTN4, POU4F3, OTP, GDNF, FYN, |
| | neurons | | | | EN1, CHAT, GSX2, OTX2, NEFM, NKX2- |
| | | | | | 8, CFTR |
| GO: 0045449 | regulation of | 42 | 4.73E−05 | 1.19E−02 | ZNF560, ZNF233, EVX2, VSX2, OTX2, |
| | transcription | | | | KLF11, MYF6, IRX5, PAX9, GSC, |
| | | | | | KLF16, EN1, SHOX2, GSX2, GRHL2, |
| | | | | | SOX7, NKX2-8, SIX6, TOX2, POU4F3, |
| | | | | | RAX, FOXD3, BCL6B, HOXD12, HOXD3, |
| | | | | | HOXD10, ZNF236, ONECUT2, ALX1, |
| | | | | | C14orf39, EVX1, HOXA1, ONECUT1, |
| | | | | | NKX2-6, OLIG3, OTP, PRDM14, DLX2, |
| | | | | | NKX6-1, HOXA10, WT1, HOXA7 |

TABLE 4
Genes methylated at the 5′ end in normal brain

| | | | | | Average P | P value scores | | | | | |
|---|---|---|---|---|---|---|---|---|---|---|---|
| chr | start | end | accession | gene_symbol | value score | N1 | N2 | N3 | N4 | N5 | N6 |
| chr1 | 886785 | 890940 | NM_032129 | PLEKHN1 | 14.97 | 14.73 | 14.26 | 18.95 | 16.13 | 13.39 | 12.36 |
| chr1 | 1256597 | 1258841 | NM_152228 | TAS1R3 | 7.66 | 10.17 | 8.94 | 7.32 | 6.49 | 7.39 | 5.65 |
| chr1 | 1557590 | 1559635 | NR_002946 | MMP23A | 9.95 | 5.89 | 10.87 | 12.47 | 12.45 | 11.30 | 6.72 |
| chr1 | 1848895 | 1850056 | NM_001003808 | C1orf222 | 13.14 | 21.44 | 11.07 | 16.57 | 15.68 | 6.68 | 7.41 |
| chr1 | 1994170 | 1995508 | NM_001033581 | PRKCZ | 6.97 | 7.88 | 6.84 | 8.28 | 5.99 | 6.77 | 6.03 |
| chr1 | 6008276 | 6009325 | NM_172130 | KCNAB2 | 8.41 | 10.52 | 8.43 | 6.98 | 11.06 | 7.36 | 6.09 |
| chr1 | 25163901 | 25164846 | NM_001031680 | RUNX3 | 10.59 | 14.26 | 12.54 | 9.43 | 10.79 | 6.05 | 10.45 |
| chr1 | 27062120 | 27062979 | NM_006142 | SFN | 8.59 | 9.93 | 7.74 | 8.18 | 7.46 | 11.59 | 6.63 |
| chr1 | 34998589 | 35000128 | NM_153212 | GJB4 | 32.79 | 28.52 | 38.88 | 41.56 | 33.17 | 34.92 | 19.66 |
| chr1 | 45860802 | 45861741 | NM_001114938 | CCDC17 | 11.04 | 14.19 | 8.53 | 10.48 | 11.09 | 13.45 | 8.47 |
| chr1 | 53696980 | 53698808 | NM_033067 | DMRTB1 | 16.60 | 8.16 | 9.53 | 17.01 | 19.67 | 21.62 | 23.62 |
| chr1 | 55043525 | 55044789 | NM_152607 | C1orf177 | 9.92 | 11.63 | 9.54 | 11.13 | 12.03 | 9.28 | 5.93 |
| chr1 | 64441283 | 64442237 | NM_152489 | UBE2U | 8.66 | 8.51 | 7.14 | 10.45 | 11.00 | 6.21 | 8.62 |
| chr1 | 68288592 | 68290258 | NM_004675 | DIRAS3 | 34.86 | 46.15 | 25.24 | 43.22 | 27.90 | 39.78 | 26.86 |
| chr1 | 92186921 | 92187877 | NM_207189 | BRDT | 15.43 | 17.69 | 9.18 | 15.99 | 18.51 | 18.79 | 12.40 |
| chr1 | 92455965 | 92456520 | NM_001012425 | C1orf146 | 12.21 | 12.28 | 13.35 | 11.96 | 10.45 | 12.41 | 12.80 |
| chr1 | 159761270 | 159762009 | NM_002155 | HSPA6 | 9.99 | 10.87 | 8.67 | 10.34 | 14.67 | 8.66 | 6.72 |
| chr1 | 165224549 | 165225393 | NM_032858 | MAEL | 13.73 | 12.09 | 11.89 | 16.17 | 14.70 | 12.71 | 14.80 |
| chr1 | 221634128 | 221634757 | NM_152610 | C1orf65 | 10.11 | 14.52 | 8.17 | 12.32 | 9.59 | 7.25 | 8.82 |
| chr1 | 226069650 | 226070909 | NM_183062 | MPN2 | 8.12 | 9.28 | 8.01 | 8.18 | 10.52 | 6.96 | 5.76 |
| chr1 | 226679129 | 226679963 | NM_003493 | HIST3H3 | 24.80 | 30.55 | 24.80 | 26.21 | 22.78 | 26.66 | 17.81 |
| chr1 | 245681115 | 245682749 | NM_001004492 | OR2B11 | 12.58 | 17.09 | 10.95 | 13.64 | 17.28 | 8.10 | 8.39 |
| chr1 | 246166331 | 246167265 | NM_175911 | OR2L13 | 9.62 | 13.82 | 8.69 | 7.55 | 9.67 | 8.37 | 9.61 |
| chr10 | 84545 | 86000 | NM_177987 | TUBB8 | 26.76 | 23.45 | 20.43 | 27.58 | 24.01 | 30.78 | 34.34 |
| chr10 | 1194985 | 1196034 | NR_015376 | C10orf139 | 35.51 | 31.12 | 35.42 | 47.77 | 38.90 | 32.47 | 27.35 |
| chr10 | 5396191 | 5397142 | NM_053049 | UCN3 | 8.37 | 11.04 | 6.07 | 7.89 | 11.89 | 7.39 | 5.91 |
| chr10 | 5530786 | 5532355 | NM_017422 | CALML5 | 26.62 | 34.88 | 22.07 | 30.92 | 28.60 | 27.12 | 16.13 |
| chr10 | 5556345 | 5557875 | NM_005185 | CALML3 | 25.87 | 30.12 | 20.99 | 27.52 | 24.20 | 29.47 | 22.90 |
| chr10 | 14856723 | 14857363 | NM_031453 | FAM107B | 7.73 | 10.25 | 7.01 | 7.39 | 10.11 | 6.09 | 5.50 |
| chr10 | 27742399 | 27743642 | NM_001034842 | PTCHD3 | 17.59 | 21.90 | 9.60 | 22.86 | 15.92 | 17.26 | 18.01 |
| chr10 | 37454095 | 37454949 | NM_052997 | ANKRD30A | 12.21 | 11.95 | 9.38 | 13.30 | 14.18 | 13.25 | 11.18 |
| chr10 | 47216440 | 47217099 | NM_001630 | ANXA8L2 | 5.84 | 5.91 | 7.23 | 5.74 | 5.68 | 5.57 | 4.92 |
| chr10 | 48447387 | 48447836 | NM_015605 | PTPN20B | 6.11 | 4.80 | 7.59 | 6.05 | 6.40 | 6.48 | 5.34 |
| chr10 | 94810041 | 94813282 | NM_183374 | CYP26C1 | 10.63 | 9.15 | 12.56 | 16.77 | 8.43 | 11.02 | 5.83 |
| chr10 | 105207770 | 105209219 | NM_001001412 | CALHM1 | 8.15 | 10.40 | 7.29 | 8.73 | 8.02 | 7.59 | 6.87 |
| chr10 | 115928219 | 115929474 | NM_198795 | TDRD1 | 17.76 | 22.47 | 16.00 | 22.82 | 18.87 | 16.13 | 10.26 |
| chr10 | 121285836 | 121286605 | NM_002925 | RGS10 | 5.73 | 6.90 | 5.00 | 5.45 | 4.86 | 5.35 | 6.79 |
| chr10 | 127453925 | 127455192 | NM_147191 | MMP21 | 8.25 | 8.96 | 7.79 | 9.38 | 9.73 | 6.54 | 7.08 |
| chr10 | 127574241 | 127575495 | NM_145235 | FANK1 | 10.62 | 8.93 | 10.89 | 11.15 | 8.44 | 10.33 | 13.99 |
| chr10 | 135086692 | 135087332 | NM_001012508 | SPRN | 6.87 | 6.86 | 8.98 | 6.67 | 7.33 | 6.07 | 5.33 |
| chr11 | 279861 | 280695 | NM_025092 | ATHL1 | 6.53 | 8.86 | 6.11 | 6.61 | 7.01 | 5.30 | 5.31 |
| chr11 | 617645 | 618084 | NM_021920 | SCT | 14.81 | 17.39 | 10.06 | 14.50 | 13.85 | 16.29 | 16.80 |
| chr11 | 820907 | 822857 | NM_001039490 | CD151 | 6.38 | 5.06 | 6.13 | 7.94 | 5.60 | 7.86 | 5.70 |
| chr11 | 1817998 | 1820162 | NM_001145841 | TNNI2 | 7.95 | 5.26 | 9.55 | 8.00 | 8.60 | 9.31 | 7.01 |
| chr11 | 1829198 | 1830654 | NM_002339 | LSP1 | 9.03 | 10.85 | 10.43 | 8.71 | 10.86 | 8.30 | 5.02 |
| chr11 | 1830796 | 1832555 | NM_002339 | LSP1 | 8.89 | 10.18 | 9.69 | 7.43 | 12.21 | 7.07 | 6.78 |
| chr11 | 1846808 | 1850966 | NM_001013255 | LSP1 | 11.19 | 10.57 | 9.06 | 12.27 | 15.93 | 12.00 | 7.33 |
| chr11 | 2379793 | 2381347 | NM_005706 | TSSC4 | 7.76 | 9.51 | 8.09 | 6.60 | 9.69 | 7.70 | 5.00 |
| chr11 | 2399707 | 2404341 | NM_014555 | TRPM5 | 5.66 | 5.89 | 6.14 | 6.36 | 5.71 | 5.29 | 4.56 |
| chr11 | 2438393 | 2442742 | NM_181798 | KCNQ1 | 7.14 | 8.31 | 6.13 | 8.02 | 8.94 | 6.87 | 4.56 |
| chr11 | 3195404 | 3196543 | NR_027138 | C11orf36 | 17.17 | 9.78 | 17.25 | 19.08 | 22.60 | 19.18 | 15.13 |
| chr11 | 3210151 | 3211310 | NM_001039165 | MRGPRE | 20.44 | 29.55 | 17.71 | 25.50 | 22.59 | 15.04 | 12.24 |
| chr11 | 6217772 | 6218421 | NM_001037329 | CNGA4 | 20.21 | 26.51 | 17.18 | 19.66 | 21.01 | 19.24 | 17.68 |
| chr11 | 6297026 | 6299075 | NM_145040 | PRKCDBP | 8.97 | 11.95 | 7.50 | 10.60 | 9.31 | 9.25 | 5.23 |
| chr11 | 17365816 | 17366370 | NM_000525 | KCNJ11 | 8.13 | 8.84 | 8.27 | 9.94 | 9.19 | 5.28 | 7.26 |
| chr11 | 60038866 | 60039824 | NM_001012417 | MS4A13 | 15.15 | 20.20 | 17.78 | 9.47 | 18.28 | 14.50 | 10.69 |
| chr11 | 63829557 | 63830496 | NM_004451 | ESRRA | 9.12 | 8.04 | 7.20 | 7.21 | 8.41 | 13.33 | 10.51 |
| chr11 | 66381905 | 66383149 | NM_024036 | LRFN4 | 9.46 | 10.98 | 8.44 | 10.91 | 9.16 | 10.08 | 7.17 |
| chr11 | 76490756 | 76492103 | NM_006189 | OMP | 16.14 | 25.55 | 13.79 | 17.01 | 16.56 | 13.44 | 10.48 |
| chr11 | 103412459 | 103413298 | NM_001001711 | DDI1 | 44.43 | 51.82 | 39.03 | 48.69 | 45.01 | 39.40 | 42.65 |
| chr11 | 111542612 | 111543474 | NM_031275 | TEX12 | 13.56 | 17.44 | 9.35 | 15.76 | 15.43 | 10.55 | 12.85 |
| chr11 | 119513243 | 119514010 | NM_012101 | TRIM29 | 17.46 | 24.68 | 17.16 | 17.34 | 18.49 | 15.64 | 11.41 |
| chr11 | 128317836 | 128318785 | NM_022112 | TP53AIP1 | 6.44 | 7.93 | 6.88 | 7.28 | 5.92 | 5.55 | 5.10 |
| chr12 | 56008 | 56752 | NM_015232 | IQSEC3 | 7.41 | 6.73 | 5.98 | 9.15 | 6.34 | 8.40 | 7.85 |
| chr12 | 4424438 | 4425797 | NM_020996 | FGF6 | 18.12 | 25.21 | 14.09 | 20.15 | 18.42 | 15.12 | 15.70 |
| chr12 | 5023862 | 5024596 | NM_002234 | KCNA5 | 15.07 | 16.02 | 14.90 | 17.75 | 14.33 | 18.36 | 9.05 |
| chr12 | 5472728 | 5474382 | NM_002527 | NTF3 | 24.04 | 22.82 | 23.82 | 23.24 | 23.26 | 27.27 | 23.84 |
| chr12 | 31835936 | 31836485 | NM_001013699 | LOC440093 | 31.58 | 32.60 | 28.81 | 33.36 | 33.59 | 27.22 | 33.90 |
| chr12 | 38305849 | 38306399 | NM_001031748 | C12orf40 | 6.41 | 8.52 | 5.37 | 5.21 | 6.56 | 4.90 | 7.88 |
| chr12 | 47462707 | 47463456 | NM_015270 | ADCY6 | 9.23 | 9.57 | 10.13 | 12.17 | 9.32 | 7.05 | 7.13 |
| chr12 | 50971013 | 50972263 | NM_002281 | KRT81 | 21.22 | 21.25 | 17.97 | 18.00 | 31.76 | 21.71 | 16.61 |
| chr12 | 50981226 | 50982470 | NM_002284 | KRT86 | 52.75 | 69.89 | 41.53 | 85.40 | 52.68 | 30.08 | 36.94 |
| chr12 | 51000856 | 51001605 | NM_002282 | KRT83 | 10.63 | 17.74 | 7.45 | 10.68 | 10.07 | 9.89 | 7.94 |
| chr12 | 51046887 | 51048336 | NM_002283 | KRT85 | 33.42 | 41.59 | 26.55 | 33.64 | 40.47 | 30.66 | 27.61 |
| chr12 | 79634253 | 79635392 | NM_005593 | MYF5 | 7.31 | 6.69 | 7.12 | 7.51 | 6.78 | 6.20 | 9.53 |
| chr12 | 89872885 | 89873850 | NM_152638 | C12orf12 | 8.14 | 9.20 | 7.82 | 9.52 | 9.53 | 6.22 | 6.56 |
| chr12 | 100656881 | 100657620 | NM_153694 | SYCP3 | 19.04 | 18.20 | 18.83 | 13.89 | 23.00 | 21.48 | 18.80 |
| chr12 | 121780904 | 121781852 | NM_032554 | GPR81 | 10.91 | 13.15 | 8.07 | 13.24 | 14.07 | 9.46 | 7.45 |
| chr13 | 18653757 | 18654506 | NM_006001 | TUBA3C | 24.48 | 29.32 | 25.28 | 24.57 | 19.70 | 25.19 | 22.84 |
| chr13 | 23780205 | 23781177 | NM_178540 | C1QTNF9 | 12.08 | 9.82 | 10.82 | 10.92 | 11.52 | 13.62 | 15.80 |
| chr13 | 24235708 | 24236472 | NM_031277 | RNF17 | 12.84 | 14.70 | 15.31 | 13.61 | 17.03 | 9.87 | 6.51 |
| chr13 | 35686378 | 35687238 | NM_017826 | SOHLH2 | 8.02 | 11.21 | 7.79 | 7.67 | 8.86 | 6.70 | 5.86 |
| chr13 | 36577620 | 36578169 | NM_145203 | CSNK1A1L | 9.61 | 11.58 | 10.79 | 8.34 | 10.80 | 8.58 | 7.54 |
| chr13 | 56612475 | 56613524 | NM_001130407 | LOC729250 | 42.01 | 47.96 | 29.61 | 63.64 | 42.30 | 37.25 | 31.30 |
| chr13 | 56618844 | 56620073 | NM_001130407 | LOC729250 | 57.64 | 65.17 | 57.90 | 59.75 | 83.82 | 33.31 | 45.89 |
| chr13 | 56625901 | 56626645 | NM_001130407 | LOC729250 | 77.38 | 82.71 | 80.66 | 83.29 | 84.52 | 64.28 | 68.83 |
| chr13 | 56631990 | 56633239 | NM_001130407 | LOC729250 | 54.01 | 62.05 | 50.62 | 58.80 | 63.80 | 57.68 | 31.09 |
| chr13 | 56638553 | 56639782 | NM_001130407 | LOC729250 | 52.13 | 58.86 | 49.60 | 66.82 | 61.00 | 43.23 | 33.29 |
| chr13 | 94883460 | 94884537 | NM_182848 | CLDN10 | 8.83 | 7.22 | 9.61 | 8.47 | 11.83 | 7.52 | 8.33 |
| chr13 | 97626570 | 97627629 | NM_178861 | RNF113B | 32.89 | 46.88 | 19.32 | 39.08 | 31.42 | 36.69 | 23.98 |
| chr13 | 113155649 | 113156614 | NM_138430 | ADPRHL1 | 13.03 | 14.48 | 20.29 | 9.81 | 11.31 | 9.94 | 12.37 |
| chr14 | 18622785 | 18623513 | NR_027480 | POTEG | 31.88 | 30.01 | 40.36 | 37.79 | 31.56 | 21.78 | 29.75 |
| chr14 | 46190425 | 46191076 | NM_080746 | RPL10L | 8.93 | 7.89 | 7.79 | 9.60 | 7.70 | 8.45 | 12.15 |
| chr14 | 59263472 | 59264928 | NM_206857 | RTN1 | 26.09 | 33.37 | 19.98 | 31.71 | 19.15 | 20.18 | 32.13 |
| chr14 | 92882593 | 92883942 | NM_182971 | COX8C | 30.34 | 16.34 | 24.90 | 25.16 | 22.78 | 51.39 | 41.50 |
| chr14 | 100262203 | 100262732 | NM_003836 | DLK1 | 11.09 | 10.57 | 12.30 | 11.27 | 9.89 | 10.37 | 12.13 |
| chr14 | 101097753 | 101098912 | NM_001362 | DIO3 | 15.63 | 12.01 | 18.23 | 15.00 | 19.59 | 16.86 | 12.08 |
| chr14 | 104266497 | 104266926 | NM_199165 | ADSSL1 | 21.77 | 17.08 | 27.02 | 23.39 | 26.43 | 16.22 | 20.45 |
| chr15 | 19336475 | 19337416 | NM_207355 | POTEB | 42.79 | 47.01 | 48.91 | 41.82 | 55.41 | 37.77 | 25.81 |
| chr15 | 20506397 | 20507346 | NM_001033028 | CYFIP1 | 20.89 | 27.04 | 21.56 | 19.67 | 26.96 | 13.88 | 16.26 |
| chr15 | 22471456 | 22472405 | NM_018958 | C15orf2 | 12.17 | 9.71 | 10.47 | 18.23 | 11.88 | 11.04 | 11.70 |
| chr15 | 22750434 | 22752183 | NM_005678 | SNURF | 30.41 | 29.24 | 23.55 | 27.41 | 37.18 | 31.03 | 34.06 |
| chr15 | 41728135 | 41729101 | NM_172097 | CATSPER2 | 28.35 | 9.42 | 28.92 | 43.02 | 36.66 | 27.49 | 24.57 |
| chr15 | 57285847 | 57286806 | NM_033195 | LDHAL6B | 14.17 | 13.16 | 14.91 | 14.06 | 12.40 | 14.72 | 15.75 |
| chr15 | 67009239 | 67010467 | NM_145658 | SPESP1 | 37.94 | 19.77 | 26.43 | 46.95 | 43.55 | 45.96 | 44.97 |
| chr15 | 71521869 | 71522920 | NM_001042367 | C15orf60 | 13.97 | 14.42 | 8.82 | 14.37 | 17.14 | 12.57 | 16.48 |
| chr15 | 72254477 | 72255116 | NM_201526 | ISLR | 20.56 | 26.49 | 22.67 | 21.15 | 26.10 | 14.24 | 12.72 |
| chr16 | 675033 | 675787 | NM_001005920 | JMJD8 | 12.92 | 14.80 | 10.24 | 14.96 | 13.55 | 11.61 | 12.34 |
| chr16 | 1068092 | 1070441 | NM_001053 | SSTR5 | 34.53 | 40.96 | 37.29 | 29.40 | 37.36 | 38.88 | 23.29 |
| chr16 | 1478290 | 1479239 | NM_001013658 | C16orf38 | 14.41 | 14.66 | 15.85 | 13.68 | 17.47 | 14.01 | 10.79 |
| chr16 | 1522860 | 1524624 | NM_024600 | TMEM204 | 9.18 | 11.92 | 7.73 | 7.63 | 9.60 | 10.22 | 8.01 |
| chr16 | 2807389 | 2808027 | NM_144957 | PRSS21 | 10.34 | 9.77 | 6.95 | 15.92 | 13.20 | 10.41 | 5.77 |
| chr16 | 3082682 | 3083229 | NM_032805 | ZSCAN10 | 5.79 | 6.51 | 6.50 | 6.69 | 5.28 | 4.66 | 5.13 |
| chr16 | 3433008 | 3434339 | NM_001083601 | NAT15 | 49.84 | 52.76 | 30.21 | 62.03 | 68.42 | 31.98 | 53.63 |
| chr16 | 3486239 | 3486788 | NM_001080524 | C16orf90 | 13.78 | 16.85 | 12.31 | 15.67 | 14.16 | 11.34 | 12.34 |
| chr16 | 32594155 | 32595099 | NM_016212 | TP53TG3 | 29.48 | 32.02 | 19.45 | 39.59 | 30.74 | 26.26 | 28.82 |
| chr16 | 33112997 | 33113931 | NM_016212 | TP53TG3 | 36.76 | 40.56 | 20.49 | 50.56 | 35.68 | 30.82 | 42.46 |
| chr16 | 33169532 | 33170466 | NM_016212 | TP53TG3 | 36.49 | 41.41 | 20.71 | 48.20 | 36.30 | 36.20 | 36.10 |
| chr16 | 45160325 | 45161269 | NR_026556 | ANKRD26P1 | 28.14 | 29.86 | 22.30 | 27.10 | 26.67 | 37.82 | 25.07 |
| chr16 | 54423975 | 54424824 | NM_001266 | CES1 | 13.58 | 18.06 | 10.29 | 15.23 | 18.75 | 8.91 | 10.22 |
| chr16 | 65156999 | 65158350 | NM_052999 | CMTM1 | 24.69 | 31.78 | 23.76 | 23.95 | 21.11 | 21.31 | 26.23 |
| chr16 | 65740729 | 65741468 | NM_033309 | B3GNT9 | 9.80 | 5.64 | 11.37 | 11.74 | 8.35 | 11.84 | 9.86 |
| chr16 | 66571411 | 66572180 | NM_001129758 | DPEP3 | 13.68 | 14.14 | 11.24 | 16.10 | 15.37 | 13.19 | 12.03 |
| chr16 | 70080556 | 70081425 | NM_006961 | ZNF19 | 14.69 | 16.51 | 14.66 | 15.45 | 13.62 | 14.45 | 13.44 |
| chr16 | 73012597 | 73013956 | NM_001011880 | CLEC18B | 6.59 | 7.74 | 6.68 | 7.05 | 7.54 | 5.79 | 4.76 |
| chr16 | 73797516 | 73798755 | NM_001025200 | CTRB2 | 5.57 | 6.80 | 6.32 | 4.54 | 5.73 | 5.20 | 4.83 |
| chr16 | 82781765 | 82782707 | NM_001145400 | ADAD2 | 14.11 | 11.48 | 14.36 | 17.08 | 14.35 | 11.97 | 15.42 |
| chr16 | 87330130 | 87330969 | NM_001142864 | FAM38A | 7.92 | 7.60 | 8.77 | 9.15 | 8.01 | 7.78 | 6.21 |
| chr17 | 2645705 | 2646634 | NM_015085 | GARNL4 | 14.28 | 17.59 | 17.08 | 10.76 | 12.46 | 18.52 | 9.25 |
| chr17 | 3321639 | 3322576 | NM_032598 | SPATA22 | 51.95 | 55.52 | 34.18 | 58.36 | 57.16 | 55.68 | 50.81 |
| chr17 | 3446892 | 3447641 | NM_018727 | TRPV1 | 7.86 | 9.45 | 9.32 | 6.62 | 8.22 | 5.26 | 8.27 |
| chr17 | 4743347 | 4746194 | NM_001145536 | LOC100130311 | 12.04 | 11.16 | 13.76 | 15.31 | 11.18 | 12.03 | 8.80 |
| chr17 | 6839343 | 6841183 | NM_000697 | ALOX12 | 6.88 | 9.54 | 5.43 | 7.80 | 7.84 | 5.69 | 5.01 |
| chr17 | 7697816 | 7699847 | NM_203411 | TMEM88 | 7.24 | 6.79 | 9.70 | 8.97 | 7.79 | 5.20 | 4.99 |
| chr17 | 9748582 | 9750053 | NM_002903 | RCVRN | 18.06 | 19.85 | 20.69 | 13.99 | 18.70 | 15.67 | 19.45 |
| chr17 | 17421328 | 17421768 | NM_148173 | PEMT | 7.16 | 8.10 | 7.27 | 6.57 | 8.21 | 5.86 | 6.93 |
| chr17 | 18468941 | 18469890 | NM_182568 | CCDC144B | 22.52 | 22.23 | 20.82 | 21.07 | 25.97 | 26.48 | 18.55 |
| chr17 | 18795404 | 18796353 | NM_152351 | SLC5A10 | 10.73 | 13.89 | 11.21 | 10.17 | 10.18 | 10.59 | 8.35 |
| chr17 | 20739556 | 20740720 | NM_001004306 | CCDC144NL | 29.77 | 30.08 | 30.31 | 29.12 | 36.76 | 29.55 | 22.82 |
| chr17 | 21748717 | 21749646 | NM_203392 | FAM27L | 15.16 | 18.01 | 12.53 | 16.83 | 18.19 | 13.17 | 12.24 |
| chr17 | 23658080 | 23658835 | NM_173624 | FLJ40504 | 6.71 | 5.39 | 6.50 | 7.75 | 7.10 | 5.64 | 7.91 |
| chr17 | 34250855 | 34251398 | NM_001080465 | C17orf98 | 10.83 | 5.23 | 12.21 | 15.31 | 13.35 | 10.62 | 8.25 |
| chr17 | 36475474 | 36476433 | NM_033184 | KRTAP2-4 | 10.88 | 8.69 | 10.10 | 13.71 | 9.85 | 11.70 | 11.20 |
| chr17 | 36725274 | 36726223 | NM_031964 | KRTAP17-1 | 19.55 | 27.44 | 14.63 | 22.33 | 24.07 | 15.44 | 13.42 |
| chr17 | 36996489 | 36997433 | NM_000526 | KRT14 | 13.48 | 15.74 | 13.74 | 13.64 | 11.94 | 14.69 | 11.14 |
| chr17 | 37034156 | 37034805 | NM_000422 | KRT17 | 6.67 | 7.86 | 5.51 | 5.86 | 7.48 | 7.43 | 5.89 |
| chr17 | 37527672 | 37528921 | NM_033194 | HSPB9 | 11.20 | 12.86 | 9.23 | 13.66 | 11.51 | 9.62 | 10.33 |
| chr17 | 39191499 | 39192250 | NM_025237 | SOST | 7.00 | 7.08 | 6.97 | 6.63 | 7.00 | 8.97 | 5.34 |
| chr17 | 49254484 | 49255423 | NM_032559 | KIF2B | 12.32 | 13.23 | 9.42 | 14.20 | 13.74 | 11.16 | 12.19 |
| chr17 | 62470387 | 62471721 | NM_000727 | CACNG1 | 22.03 | 25.91 | 22.92 | 18.22 | 28.56 | 24.30 | 12.28 |
| chr17 | 71815097 | 71816147 | NM_032134 | QRICH2 | 34.88 | 25.65 | 31.36 | 32.78 | 39.30 | 42.92 | 37.29 |
| chr17 | 75766293 | 75767047 | NM_024110 | CARD14 | 11.13 | 11.97 | 11.32 | 8.22 | 16.82 | 7.37 | 11.09 |
| chr17 | 75927527 | 75928476 | NM_020914 | RNF213 | 8.67 | 10.87 | 6.06 | 10.11 | 7.92 | 11.12 | 5.93 |
| chr17 | 77109741 | 77110865 | NM_012418 | FSCN2 | 15.82 | 17.27 | 13.30 | 17.84 | 17.34 | 15.34 | 13.80 |
| chr17 | 77386013 | 77386662 | NM_001007533 | DYSFIP1 | 8.51 | 13.55 | 8.12 | 7.60 | 10.08 | 6.29 | 5.41 |
| chr17 | 77473624 | 77474153 | NM_032711 | MAFG | 11.26 | 7.11 | 8.03 | 16.55 | 10.93 | 14.55 | 10.40 |
| chr17 | 77925511 | 77926855 | NM_018949 | UTS2R | 10.29 | 7.32 | 12.90 | 10.94 | 12.78 | 10.75 | 7.07 |
| chr18 | 569985 | 570939 | NM_004066 | CETN1 | 40.99 | 11.88 | 45.66 | 55.56 | 50.67 | 39.81 | 42.34 |
| chr18 | 13815665 | 13816754 | NM_005913 | MC5R | 24.82 | 31.04 | 25.88 | 21.22 | 35.13 | 17.21 | 18.47 |
| chr18 | 18251681 | 18252650 | NM_172241 | CTAGE1 | 17.99 | 19.71 | 16.34 | 24.18 | 24.73 | 8.77 | 14.19 |
| chr18 | 21967313 | 21968170 | NM_001025096 | PSMA8 | 17.28 | 19.59 | 17.74 | 16.87 | 17.36 | 14.62 | 17.52 |
| chr18 | 27594502 | 27595268 | NM_001034172 | MCART2 | 19.93 | 24.74 | 16.81 | 24.53 | 21.00 | 12.87 | 19.65 |
| chr18 | 42802973 | 42805327 | NM_145653 | TCEB3C | 134.12 | 138.86 | 129.94 | 145.26 | 146.36 | 120.71 | 123.57 |
| chr18 | 42808886 | 42811242 | NM_145653 | TCEB3C | 130.36 | 139.99 | 125.33 | 143.84 | 141.45 | 130.20 | 101.36 |
| chr18 | 42815025 | 42816781 | NM_016427 | TCEB3B | 44.78 | 43.66 | 36.25 | 44.78 | 43.21 | 52.83 | 47.94 |
| chr18 | 46062462 | 46063396 | NM_015844 | MBD1 | 11.75 | 21.26 | 8.42 | 11.86 | 8.59 | 8.94 | 11.42 |
| chr18 | 59294602 | 59295373 | NM_002639 | SERPINB5 | 7.41 | 8.82 | 5.33 | 8.85 | 6.76 | 7.45 | 7.23 |
| chr18 | 75723859 | 75725028 | NM_012283 | KCNG2 | 23.25 | 23.66 | 19.82 | 15.02 | 26.45 | 26.68 | 27.88 |
| chr19 | 425784 | 426743 | NM_182577 | ODF3L2 | 8.57 | 9.13 | 7.41 | 8.54 | 13.27 | 6.26 | 6.78 |
| chr19 | 1904572 | 1905206 | NM_152771 | C19orf34 | 10.82 | 12.33 | 10.89 | 13.22 | 11.30 | 10.70 | 6.47 |
| chr19 | 3129270 | 3130838 | NM_003775 | S1PR4 | 10.84 | 5.71 | 9.59 | 10.85 | 14.04 | 16.52 | 8.36 |
| chr19 | 4743132 | 4744201 | NM_018708 | FEM1A | 18.37 | 21.90 | 13.30 | 17.30 | 20.23 | 23.20 | 14.30 |
| chr19 | 5405666 | 5407310 | NM_181710 | ZNRF4 | 24.45 | 25.36 | 27.26 | 24.38 | 22.44 | 25.60 | 21.66 |
| chr19 | 5735522 | 5736971 | NM_001134316 | MGC24975 | 8.62 | 9.66 | 5.81 | 10.76 | 9.82 | 8.84 | 6.83 |
| chr19 | 7652515 | 7653759 | NM_001042461 | TRAPPC5 | 14.36 | 8.56 | 15.54 | 12.75 | 17.37 | 23.49 | 8.48 |
| chr19 | 8668814 | 8669778 | NM_178525 | ACTL9 | 15.26 | 23.38 | 9.71 | 14.47 | 15.21 | 13.45 | 15.34 |
| chr19 | 8794380 | 8795329 | NM_144693 | ZNF558 | 18.84 | 28.56 | 16.40 | 14.72 | 22.21 | 13.46 | 17.68 |
| chr19 | 10081535 | 10082279 | NM_002566 | P2RY11 | 22.27 | 31.42 | 20.97 | 27.40 | 25.50 | 17.83 | 10.48 |
| chr19 | 10082415 | 10083249 | NM_002566 | P2RY11 | 12.04 | 16.24 | 10.64 | 15.02 | 12.36 | 11.88 | 6.13 |
| chr19 | 15699554 | 15700503 | NM_013939 | OR10H2 | 23.49 | 18.35 | 21.20 | 31.69 | 26.08 | 21.19 | 22.44 |
| chr19 | 15779233 | 15780184 | NM_013940 | OR10H1 | 29.97 | 40.31 | 29.73 | 34.01 | 27.70 | 29.10 | 18.96 |
| chr19 | 16861645 | 16862379 | NM_003950 | F2RL3 | 14.99 | 15.14 | 12.73 | 17.78 | 19.96 | 15.40 | 8.95 |
| chr19 | 17348683 | 17349427 | NM_031310 | PLVAP | 12.02 | 14.18 | 11.19 | 11.93 | 9.23 | 11.86 | 13.71 |
| chr19 | 18406231 | 18409280 | NM_016368 | ISYNA1 | 9.47 | 11.10 | 12.24 | 9.39 | 7.89 | 8.91 | 7.27 |
| chr19 | 19485956 | 19487705 | NM_015965 | NDUFA13 | 23.44 | 19.43 | 23.04 | 29.97 | 24.90 | 27.36 | 15.96 |
| chr19 | 37902256 | 37902885 | NM_001110822 | TDRD12 | 10.24 | 15.30 | 7.33 | 9.64 | 10.82 | 12.05 | 6.31 |
| chr19 | 40533503 | 40535052 | NM_005303 | FFAR1 | 15.08 | 23.03 | 14.60 | 13.11 | 16.61 | 13.06 | 10.09 |
| chr19 | 40631857 | 40632626 | NM_005306 | FFAR2 | 6.71 | 8.06 | 6.73 | 5.84 | 7.21 | 5.50 | 6.94 |
| chr19 | 43568464 | 43569908 | NM_152657 | GGN | 11.85 | 12.47 | 12.93 | 12.05 | 14.12 | 11.55 | 7.96 |
| chr19 | 45587124 | 45588088 | NM_144685 | HIPK4 | 11.21 | 9.64 | 13.78 | 12.74 | 15.97 | 6.93 | 8.22 |
| chr19 | 47040148 | 47041188 | NM_001040283 | DMRTC2 | 16.20 | 18.08 | 16.69 | 15.23 | 16.11 | 11.39 | 19.71 |
| chr19 | 51079188 | 51080617 | NM_015649 | IRF2BP1 | 15.00 | 18.29 | 12.57 | 18.66 | 20.94 | 12.00 | 7.54 |
| chr19 | 51688686 | 51690541 | NM_020709 | PNMAL2 | 10.75 | 14.00 | 8.27 | 8.41 | 11.44 | 11.36 | 11.01 |
| chr19 | 51818645 | 51819269 | NM_000960 | PTGIR | 8.01 | 9.63 | 6.81 | 9.42 | 8.55 | 7.21 | 6.46 |
| chr19 | 53656820 | 53657569 | NM_170720 | KCNJ14 | 21.31 | 20.41 | 19.84 | 23.04 | 20.77 | 28.49 | 15.28 |
| chr19 | 54219222 | 54220176 | NM_000737 | CGB | 16.15 | 25.16 | 10.36 | 15.16 | 21.89 | 12.30 | 12.04 |
| chr19 | 54226148 | 54226592 | NM_033378 | CGB2 | 8.59 | 8.63 | 8.80 | 8.42 | 9.37 | 7.40 | 8.90 |
| chr19 | 54238117 | 54239086 | NM_033043 | CGB5 | 13.38 | 17.27 | 13.14 | 13.80 | 13.99 | 12.35 | 9.74 |
| chr19 | 54243991 | 54244945 | NM_033183 | CGB8 | 17.15 | 26.46 | 18.04 | 14.57 | 22.24 | 11.20 | 10.40 |
| chr19 | 54520006 | 54520660 | NM_014037 | SLC6A16 | 11.21 | 13.74 | 11.25 | 10.76 | 12.45 | 10.06 | 9.00 |
| chr19 | 54583019 | 54583463 | NM_144688 | CCDC155 | 11.64 | 13.18 | 11.79 | 10.85 | 11.00 | 13.09 | 9.92 |
| chr19 | 55911639 | 55912709 | NM_016148 | SHANK1 | 8.56 | 11.19 | 7.77 | 10.46 | 8.58 | 8.24 | 5.11 |
| chr19 | 56197491 | 56198350 | NM_144505 | KLK8 | 13.02 | 13.57 | 12.16 | 6.85 | 24.82 | 10.45 | 10.28 |
| chr19 | 58486057 | 58486682 | NM_033341 | BIRC8 | 14.14 | 12.38 | 10.66 | 16.11 | 11.19 | 16.70 | 17.81 |
| chr19 | 58732640 | 58733684 | NM_001079906 | ZNF331 | 34.46 | 43.47 | 23.48 | 34.91 | 37.96 | 27.88 | 39.08 |
| chr19 | 58749250 | 58750079 | NM_001079907 | ZNF331 | 36.74 | 39.92 | 17.81 | 48.06 | 27.86 | 40.94 | 45.89 |
| chr19 | 60684392 | 60687751 | NM_020378 | NAT14 | 11.78 | 11.77 | 14.86 | 10.43 | 10.57 | 13.11 | 9.93 |
| chr19 | 61431236 | 61432273 | NM_024303 | ZSCAN5A | 12.18 | 10.85 | 13.96 | 15.95 | 13.91 | 9.26 | 9.17 |
| chr19 | 62043095 | 62044373 | NR_024059 | MIMT1 | 15.56 | 9.37 | 10.07 | 24.11 | 18.27 | 20.53 | 11.01 |
| chr19 | 62321971 | 62322600 | NM_020903 | USP29 | 14.56 | 19.05 | 7.49 | 16.71 | 9.10 | 20.35 | 14.65 |
| chr19 | 62433513 | 62434437 | NM_001015878 | AURKC | 18.25 | 20.25 | 21.05 | 17.40 | 16.85 | 15.73 | 18.19 |
| chr2 | 10970124 | 10971578 | NM_002236 | KCNF1 | 27.57 | 35.95 | 25.50 | 22.03 | 35.35 | 27.97 | 18.63 |
| chr2 | 24250685 | 24252441 | NM_001040710 | C2orf84 | 8.45 | 7.67 | 6.82 | 10.45 | 9.20 | 6.90 | 9.67 |
| chr2 | 27226289 | 27226938 | NM_175769 | TCF23 | 15.75 | 15.05 | 15.45 | 13.37 | 13.20 | 16.32 | 21.11 |
| chr2 | 27351312 | 27351951 | NM_173650 | DNAJC5G | 9.14 | 10.47 | 10.01 | 6.46 | 10.06 | 11.69 | 6.17 |
| chr2 | 48697663 | 48698722 | NM_172196 | GTF2A1L | 15.52 | 18.43 | 10.58 | 20.38 | 19.67 | 14.11 | 9.93 |
| chr2 | 54336335 | 54337297 | NM_001003937 | TSPYL6 | 16.41 | 22.39 | 20.92 | 15.91 | 15.12 | 9.41 | 14.72 |
| chr2 | 84370521 | 84371670 | NR_003663 | LOC388965 | 39.46 | 51.89 | 36.84 | 42.03 | 36.91 | 30.24 | 38.85 |
| chr2 | 94900282 | 94901626 | NM_144705 | TEKT4 | 15.42 | 22.17 | 14.94 | 13.43 | 18.51 | 14.64 | 8.84 |
| chr2 | 96063947 | 96065201 | NM_207328 | LOC150763 | 55.70 | 68.78 | 50.78 | 52.54 | 61.79 | 52.91 | 47.43 |
| chr2 | 96145156 | 96146095 | NM_000682 | ADRA2B | 9.16 | 4.83 | 9.49 | 10.65 | 12.47 | 11.29 | 6.23 |
| chr2 | 97716522 | 97717066 | NM_207519 | ZAP70 | 13.84 | 13.90 | 18.74 | 12.51 | 13.22 | 12.61 | 12.03 |
| chr2 | 97717222 | 97718571 | NM_207519 | ZAP70 | 15.12 | 16.57 | 19.65 | 14.54 | 13.86 | 14.33 | 11.75 |
| chr2 | 105224921 | 105225570 | NM_007227 | GPR45 | 52.58 | 57.22 | 40.22 | 63.81 | 57.11 | 56.10 | 40.99 |
| chr2 | 108702582 | 108703121 | NM_006267 | RANBP2 | 13.69 | 14.48 | 11.27 | 13.74 | 11.08 | 14.62 | 16.97 |
| chr2 | 109909568 | 109910007 | NM_005054 | RGPD5 | 11.05 | 8.93 | 9.90 | 12.92 | 8.77 | 10.96 | 14.80 |
| chr2 | 113972455 | 113974501 | NM_012184 | FOXD4L1 | 8.97 | 8.96 | 7.68 | 9.14 | 9.89 | 9.21 | 8.95 |
| chr2 | 127679614 | 127680288 | NM_001001665 | CYP27C1 | 7.19 | 7.01 | 5.90 | 7.70 | 8.54 | 6.68 | 7.33 |
| chr2 | 130672072 | 130672916 | NM_207312 | TUBA3E | 32.99 | 51.31 | 51.63 | 35.18 | 7.97 | 6.77 | 45.08 |
| chr2 | 131691617 | 131692543 | NM_001083538 | POTEE | 33.43 | 32.45 | 39.31 | 33.73 | 27.25 | 33.52 | 34.30 |
| chr2 | 131949675 | 131950614 | NM_080386 | TUBA3D | 32.45 | 38.45 | 37.35 | 36.38 | 34.18 | 26.17 | 22.18 |
| chr2 | 132891446 | 132892097 | NM_001508 | GPR39 | 10.53 | 18.07 | 6.37 | 9.90 | 9.93 | 8.50 | 10.40 |
| chr2 | 198358003 | 198359552 | NM_197970 | BOLL | 17.79 | 12.01 | 10.70 | 17.83 | 17.73 | 19.97 | 28.50 |
| chr2 | 219961021 | 219961580 | NM_012100 | DNPEP | 7.33 | 6.58 | 8.34 | 6.30 | 6.46 | 7.28 | 9.02 |
| chr2 | 232952384 | 232953053 | NM_001632 | ALPP | 18.16 | 18.22 | 16.24 | 18.67 | 14.64 | 22.65 | 18.53 |
| chr2 | 232980448 | 232981097 | NM_031313 | ALPPL2 | 21.79 | 25.63 | 20.09 | 18.23 | 23.31 | 29.65 | 13.82 |
| chr2 | 234316887 | 234318242 | NM_001001394 | DNAJB3 | 39.49 | 45.91 | 43.13 | 39.57 | 46.90 | 35.98 | 25.44 |
| chr2 | 238371764 | 238372800 | NM_001080504 | RBM44 | 28.42 | 7.37 | 28.48 | 40.50 | 38.17 | 23.95 | 32.02 |
| chr2 | 238713572 | 238714819 | NM_198582 | KLHL30 | 21.75 | 30.96 | 20.71 | 21.12 | 25.44 | 14.63 | 17.65 |
| chr2 | 241218250 | 241218894 | NM_005301 | GPR35 | 15.19 | 16.38 | 16.96 | 17.77 | 14.29 | 15.30 | 10.43 |
| chr20 | 4176544 | 4177393 | NM_000678 | ADRA1D | 10.90 | 6.25 | 12.43 | 12.00 | 11.32 | 14.71 | 8.69 |
| chr20 | 22963737 | 22964890 | NM_001052 | SSTR4 | 10.83 | 10.15 | 8.62 | 13.86 | 6.83 | 12.97 | 12.52 |
| chr20 | 23293995 | 23294644 | NM_022482 | GZF1 | 30.99 | 39.75 | 24.66 | 31.97 | 33.79 | 30.82 | 24.93 |
| chr20 | 23915334 | 23916273 | NM_178312 | GGTLC1 | 7.19 | 7.63 | 7.12 | 8.95 | 8.55 | 5.84 | 5.07 |
| chr20 | 23917142 | 23917891 | NM_178311 | GGTLC1 | 18.89 | 26.49 | 16.64 | 18.00 | 20.04 | 14.95 | 17.24 |
| chr20 | 33343147 | 33344486 | NM_178468 | FAM83C | 17.75 | 8.15 | 18.71 | 21.19 | 24.22 | 16.83 | 17.40 |
| chr20 | 35582039 | 35583589 | NM_005386 | NNAT | 33.72 | 40.05 | 25.96 | 34.31 | 40.40 | 35.40 | 26.20 |
| chr20 | 41575691 | 41577158 | NM_015478 | L3MBTL | 12.63 | 18.43 | 10.95 | 12.71 | 15.03 | 10.69 | 7.96 |
| chr20 | 42417079 | 42418028 | NM_175914 | HNF4A | 7.53 | 10.97 | 6.28 | 7.09 | 7.82 | 6.95 | 6.07 |
| chr20 | 54541229 | 54541876 | NM_001013646 | C20orf107 | 6.65 | 7.80 | 6.67 | 5.37 | 5.94 | 6.49 | 7.63 |
| chr20 | 54633696 | 54636839 | NM_003222 | TFAP2C | 8.83 | 8.99 | 6.84 | 7.11 | 7.72 | 15.10 | 7.24 |
| chr20 | 55337555 | 55338610 | NM_198265 | SPO11 | 9.75 | 11.00 | 9.59 | 13.29 | 8.70 | 8.18 | 7.74 |
| chr20 | 56847401 | 56850580 | NM_016592 | GNAS | 31.50 | 38.68 | 26.05 | 35.15 | 22.58 | 35.61 | 30.92 |
| chr20 | 56859948 | 56860603 | NM_080425 | GNAS | 11.51 | 12.34 | 8.53 | 14.46 | 13.16 | 10.98 | 9.60 |
| chr20 | 56860909 | 56861548 | NM_080425 | GNAS | 17.66 | 27.42 | 13.40 | 24.57 | 17.10 | 10.40 | 13.09 |
| chr20 | 56861712 | 56864669 | NM_080425 | GNAS | 19.16 | 15.77 | 15.22 | 22.46 | 20.08 | 21.47 | 19.95 |
| chr20 | 57199799 | 57201264 | NM_178457 | ZNF831 | 11.74 | 6.05 | 10.06 | 16.51 | 16.27 | 11.77 | 9.78 |
| chr20 | 57941206 | 57941955 | NM_014258 | SYCP2 | 8.65 | 5.32 | 7.35 | 12.59 | 8.64 | 9.73 | 8.25 |
| chr20 | 61963314 | 61964663 | NM_080622 | C20orf135 | 12.67 | 9.75 | 10.65 | 11.76 | 20.18 | 13.41 | 10.30 |
| chr20 | 62207820 | 62209374 | NM_005286 | NPBWR2 | 26.26 | 24.42 | 22.47 | 24.66 | 24.57 | 27.27 | 34.18 |
| chr21 | 10120617 | 10121645 | NM_001187 | BAGE | 7.99 | 10.81 | 7.54 | 9.12 | 6.10 | 5.37 | 9.01 |
| chr21 | 13903590 | 13904521 | NM_174981 | POTED | 51.34 | 46.21 | 57.76 | 45.85 | 60.93 | 54.30 | 42.99 |
| chr21 | 13974048 | 13976108 | NM_001013729 | LOC441956 | 77.69 | 73.54 | 92.14 | 65.11 | 66.59 | 89.88 | 78.90 |
| chr21 | 38414860 | 38415509 | NR_026840 | DSCR8 | 9.00 | 10.15 | 6.06 | 9.55 | 10.79 | 8.16 | 9.31 |
| chr21 | 42659517 | 42660181 | NM_003225 | TFF1 | 5.79 | 7.63 | 5.57 | 5.56 | 5.35 | 4.74 | 5.90 |
| chr21 | 43902099 | 43903348 | NM_015056 | RRP1B | 23.59 | 22.42 | 21.15 | 20.41 | 24.32 | 22.09 | 31.16 |
| chr21 | 44700420 | 44701794 | NM_030891 | LRRC3 | 18.18 | 21.41 | 17.73 | 23.50 | 18.77 | 11.36 | 16.32 |
| chr21 | 44784324 | 44785274 | NM_198691 | KRTAP10-1 | 7.13 | 6.09 | 7.57 | 5.78 | 9.30 | 6.57 | 7.46 |
| chr21 | 45700562 | 45701211 | NM_030582 | COL18A1 | 11.83 | 10.72 | 15.92 | 10.55 | 12.80 | 9.86 | 11.14 |
| chr22 | 14667762 | 14668589 | NM_001136213 | POTEH | 21.26 | 21.47 | 22.71 | 26.29 | 16.20 | 19.41 | 21.44 |
| chr22 | 15868297 | 15869926 | NM_001037814 | GAB4 | 11.96 | 15.42 | 10.68 | 10.10 | 13.77 | 12.68 | 9.11 |
| chr22 | 17499105 | 17499734 | NM_053006 | TSSK2 | 25.77 | 34.81 | 25.99 | 21.26 | 33.82 | 17.68 | 21.03 |
| chr22 | 18089100 | 18094254 | NM_000407 | GP1BB | 32.20 | 31.19 | 36.88 | 36.43 | 40.87 | 28.46 | 19.39 |
| chr22 | 21317982 | 21318951 | NM_199127 | GGTLC2 | 7.16 | 7.95 | 6.81 | 9.86 | 7.33 | 5.39 | 5.60 |
| chr22 | 24467539 | 24468284 | NM_032608 | MYO18B | 10.70 | 14.80 | 12.67 | 13.04 | 10.09 | 6.91 | 6.67 |
| chr22 | 28033113 | 28034747 | NM_152237 | GAS2L1 | 7.52 | 9.44 | 7.26 | 7.05 | 7.12 | 7.69 | 6.59 |
| chr22 | 35231985 | 35232930 | NM_001102371 | FOXRED2 | 14.68 | 13.97 | 10.80 | 18.14 | 18.55 | 14.52 | 12.14 |
| chr22 | 40425282 | 40426136 | NM_152513 | MEI1 | 19.72 | 17.93 | 18.81 | 22.21 | 28.01 | 19.62 | 11.75 |
| chr22 | 43271073 | 43272028 | NM_032287 | LDOC1L | 10.60 | 10.75 | 11.41 | 10.09 | 11.50 | 10.33 | 9.49 |
| chr22 | 45307998 | 45310932 | NM_014246 | CELSR1 | 70.96 | 93.81 | 68.97 | 73.16 | 72.61 | 82.10 | 35.09 |
| chr22 | 45400538 | 45401974 | NM_015124 | GRAMD4 | 17.30 | 20.44 | 18.09 | 18.08 | 17.13 | 15.77 | 14.26 |
| chr22 | 48871561 | 48872205 | NM_018995 | MOV10L1 | 10.14 | 12.47 | 9.47 | 13.11 | 9.86 | 8.70 | 7.22 |
| chr22 | 49099931 | 49100765 | NM_001001794 | FAM116B | 5.91 | 5.65 | 5.29 | 6.21 | 5.47 | 6.88 | 5.94 |
| chr3 | 10124574 | 10125513 | NM_173472 | C3orf24 | 12.85 | 17.07 | 9.59 | 15.85 | 16.80 | 9.48 | 8.33 |
| chr3 | 14835016 | 14835970 | NM_152536 | FGD5 | 7.71 | 8.35 | 6.89 | 7.29 | 10.79 | 5.16 | 7.77 |
| chr3 | 16621582 | 16622837 | NM_001351 | DAZL | 21.35 | 16.87 | 15.93 | 32.41 | 18.72 | 22.63 | 21.52 |
| chr3 | 42701821 | 42702885 | NM_152393 | KBTBD5 | 8.83 | 9.26 | 5.65 | 10.79 | 12.31 | 8.79 | 6.19 |
| chr3 | 46849902 | 46851347 | NM_182702 | TESSP2 | 29.30 | 27.93 | 31.12 | 33.01 | 28.33 | 29.65 | 25.75 |
| chr3 | 48673342 | 48674613 | NM_001407 | CELSR3 | 9.92 | 11.56 | 8.04 | 12.43 | 8.08 | 9.26 | 10.14 |
| chr3 | 49730772 | 49731516 | NM_198722 | AMIGO3 | 10.00 | 12.08 | 7.70 | 11.18 | 9.25 | 13.20 | 6.59 |
| chr3 | 49914941 | 49915590 | NM_002447 | MST1R | 10.55 | 15.02 | 11.17 | 10.62 | 11.91 | 8.19 | 6.38 |
| chr3 | 110319501 | 110320450 | NM_014429 | MORC1 | 24.23 | 26.10 | 24.02 | 27.27 | 26.25 | 19.43 | 22.30 |
| chr3 | 120346987 | 120348436 | NM_152539 | C3orf30 | 7.48 | 9.20 | 4.57 | 8.62 | 8.71 | 9.11 | 4.64 |
| chr3 | 128189606 | 128191165 | NM_032242 | PLXNA1 | 15.39 | 14.53 | 17.35 | 13.89 | 14.92 | 18.07 | 13.61 |
| chr3 | 129668583 | 129669442 | NM_153330 | DNAJB8 | 6.38 | 7.34 | 6.07 | 7.04 | 6.55 | 4.68 | 6.60 |
| chr3 | 139210899 | 139212158 | NM_016369 | CLDN18 | 18.57 | 23.04 | 14.67 | 22.74 | 21.72 | 18.03 | 11.20 |
| chr3 | 140221409 | 140222877 | NM_001013650 | LOC389151 | 41.01 | 28.39 | 38.95 | 36.78 | 36.11 | 56.21 | 49.62 |
| chr3 | 140245379 | 140247062 | NM_001134657 | FLJ46210 | 37.02 | 18.35 | 28.21 | 44.87 | 32.74 | 53.71 | 44.25 |
| chr3 | 142979153 | 142980397 | NM_139209 | GRK7 | 23.34 | 28.43 | 20.20 | 29.03 | 26.26 | 21.24 | 14.90 |
| chr3 | 144163847 | 144164576 | NM_198504 | PAQR9 | 18.52 | 20.01 | 19.16 | 19.07 | 16.48 | 18.59 | 17.82 |
| chr3 | 185300280 | 185300841 | NM_182589 | HTR3E | 12.08 | 15.07 | 9.43 | 16.89 | 12.85 | 9.18 | 9.04 |
| chr3 | 197549449 | 197550093 | NM_138461 | TM4SF19 | 9.74 | 8.82 | 10.20 | 9.90 | 9.55 | 8.73 | 11.24 |
| chr4 | 42930 | 43789 | NM_001039127 | ZNF718 | 39.98 | 41.93 | 39.58 | 46.85 | 41.26 | 34.88 | 35.37 |
| chr4 | 608783 | 610032 | NM_000283 | PDE6B | 10.38 | 14.95 | 8.37 | 10.13 | 12.11 | 9.33 | 7.41 |
| chr4 | 688156 | 688920 | NM_006315 | PCGF3 | 17.69 | 29.34 | 14.15 | 25.33 | 17.81 | 10.18 | 9.34 |
| chr4 | 3219787 | 3221216 | NM_001012982 | C4orf44 | 15.06 | 8.40 | 15.67 | 17.28 | 16.55 | 18.19 | 14.30 |
| chr4 | 9392324 | 9394073 | NM_000798 | DRD5 | 42.67 | 43.41 | 34.80 | 47.18 | 43.39 | 43.82 | 43.39 |
| chr4 | 41678341 | 41679190 | NM_001029955 | WDR21B | 40.08 | 40.97 | 41.45 | 42.09 | 35.32 | 40.79 | 39.85 |
| chr4 | 89837818 | 89838760 | NM_153757 | NAP1L5 | 16.90 | 37.22 | 11.46 | 16.38 | 14.75 | 11.12 | 10.48 |
| chr4 | 91978847 | 91979495 | NM_183049 | TMSL3 | 8.39 | 9.02 | 7.07 | 11.97 | 6.73 | 9.24 | 6.31 |
| chr4 | 111616373 | 111617632 | NM_001977 | ENPEP | 16.69 | 17.61 | 20.70 | 14.01 | 19.59 | 15.78 | 12.45 |
| chr4 | 123072986 | 123073715 | NM_003305 | TRPC3 | 33.69 | 35.20 | 28.67 | 50.78 | 37.69 | 23.47 | 26.35 |
| chr4 | 123519326 | 123520290 | NM_001159285 | ADAD1 | 34.23 | 43.17 | 29.98 | 40.11 | 36.10 | 27.17 | 28.84 |
| chr4 | 128870244 | 128871472 | NM_031291 | SLC25A31 | 42.12 | 45.89 | 31.83 | 35.68 | 55.24 | 45.25 | 38.81 |
| chr4 | 151723471 | 151724530 | NM_006439 | MAB21L2 | 12.87 | 17.24 | 14.10 | 13.78 | 11.54 | 13.09 | 7.45 |
| chr4 | 155921173 | 155922811 | NM_144979 | RBM46 | 38.75 | 28.10 | 28.66 | 52.28 | 40.85 | 43.39 | 39.23 |
| chr4 | 164613968 | 164615243 | NM_032136 | TKTL2 | 25.70 | 31.88 | 16.28 | 27.64 | 23.60 | 26.10 | 28.72 |
| chr4 | 191098183 | 191099517 | NM_004477 | FRG1 | 12.98 | 9.68 | 10.66 | 13.98 | 12.28 | 12.98 | 18.27 |
| chr4 | 191142243 | 191143786 | NM_020040 | TUBB4Q | 24.43 | 27.81 | 24.37 | 31.81 | 26.05 | 19.51 | 17.06 |
| chr5 | 244817 | 245561 | NM_001080478 | LOC389257 | 5.97 | 5.16 | 6.12 | 5.96 | 6.56 | 6.81 | 5.22 |
| chr5 | 1254190 | 1255119 | NM_001003841 | SLC6A19 | 7.07 | 7.45 | 6.99 | 6.48 | 8.26 | 8.15 | 5.08 |
| chr5 | 1345979 | 1347540 | NM_198255 | TERT | 16.69 | 18.46 | 18.32 | 18.33 | 14.84 | 21.08 | 9.07 |
| chr5 | 55069354 | 55070737 | NM_001136034 | DDX4 | 23.01 | 26.75 | 17.21 | 23.30 | 22.58 | 25.53 | 22.70 |
| chr5 | 101862110 | 101863456 | NM_173488 | SLCO6A1 | 8.79 | 8.43 | 8.06 | 9.00 | 13.43 | 6.48 | 7.31 |
| chr5 | 121214963 | 121216013 | NM_177478 | FTMT | 19.38 | 12.09 | 17.31 | 23.23 | 22.13 | 17.32 | 24.18 |
| chr5 | 135720291 | 135721827 | NM_020389 | TRPC7 | 36.13 | 31.74 | 33.43 | 33.27 | 36.43 | 35.60 | 46.30 |
| chr5 | 140032247 | 140034004 | NM_194249 | DND1 | 12.88 | 13.55 | 16.66 | 12.73 | 17.44 | 9.92 | 6.99 |
| chr5 | 140663339 | 140663988 | NM_031947 | SLC25A2 | 6.85 | 6.80 | 6.72 | 7.52 | 7.55 | 6.87 | 5.63 |
| chr5 | 140699090 | 140699739 | NM_018915 | PCDHGA2 | 12.25 | 11.25 | 19.91 | 8.71 | 13.72 | 13.53 | 6.36 |
| chr5 | 140752377 | 140753116 | NM_032088 | PCDHGA8 | 6.51 | 5.93 | 5.05 | 7.79 | 6.55 | 8.22 | 5.52 |
| chr5 | 140790699 | 140792828 | NM_003735 | PCDHGA12 | 19.41 | 16.52 | 14.46 | 23.15 | 17.32 | 33.40 | 11.58 |
| chr5 | 149525754 | 149527118 | NM_001804 | CDX1 | 9.49 | 10.53 | 7.67 | 9.99 | 8.81 | 7.44 | 12.54 |
| chr5 | 157010923 | 157012370 | NM_007017 | SOX30 | 8.87 | 9.41 | 7.07 | 11.83 | 10.85 | 6.58 | 7.46 |
| chr5 | 169464997 | 169466263 | NM_144769 | FOXI1 | 8.68 | 9.08 | 8.77 | 8.37 | 11.06 | 9.80 | 5.00 |
| chr5 | 175041640 | 175042484 | NM_022304 | HRH2 | 5.93 | 6.93 | 5.74 | 7.88 | 4.49 | 4.39 | 6.15 |
| chr6 | 601409 | 602763 | NM_148959 | HUS1B | 26.93 | 29.09 | 24.65 | 31.45 | 24.57 | 24.79 | 27.02 |
| chr6 | 3793831 | 3796095 | NM_012135 | FAM50B | 28.32 | 48.91 | 10.94 | 39.48 | 21.57 | 24.69 | 24.36 |
| chr6 | 22676880 | 22678720 | NM_138574 | HDGFL1 | 52.66 | 73.92 | 37.83 | 68.32 | 48.13 | 44.60 | 43.16 |
| chr6 | 25834225 | 25835591 | NM_170610 | HIST1H2BA | 23.66 | 27.27 | 22.72 | 19.92 | 29.03 | 26.30 | 16.73 |
| chr6 | 26354608 | 26355450 | NM_003547 | HIST1H4G | 23.76 | 27.28 | 23.68 | 26.93 | 28.01 | 16.90 | 19.77 |
| chr6 | 29503310 | 29504241 | NM_013937 | OR11A1 | 6.79 | 7.98 | 7.66 | 7.89 | 5.89 | 6.91 | 4.41 |
| chr6 | 31799061 | 31799910 | NM_138272 | C6orf25 | 9.72 | 10.84 | 9.99 | 10.13 | 12.35 | 7.67 | 7.37 |
| chr6 | 31891732 | 31893176 | NM_005345 | HSPA1A | 13.53 | 19.66 | 9.92 | 12.55 | 14.64 | 17.46 | 6.91 |
| chr6 | 46763032 | 46764676 | NM_001010870 | TDRD6 | 19.97 | 10.43 | 11.71 | 21.23 | 27.56 | 29.47 | 19.39 |
| chr6 | 74120257 | 74121520 | NM_001025290 | DPPA5 | 18.02 | 19.56 | 25.53 | 18.75 | 12.81 | 15.57 | 15.91 |
| chr6 | 74128640 | 74129269 | NM_001017361 | C6orf221 | 9.75 | 12.28 | 10.70 | 9.06 | 11.83 | 7.00 | 7.64 |
| chr6 | 74135930 | 74136609 | NM_001080507 | OOEP | 15.83 | 18.38 | 12.19 | 15.05 | 15.64 | 18.60 | 15.15 |
| chr6 | 74161006 | 74161740 | NM_018665 | DDX43 | 19.23 | 20.89 | 17.30 | 19.09 | 12.34 | 25.26 | 20.48 |
| chr6 | 88813549 | 88814678 | NM_030960 | SPACA1 | 42.20 | 51.39 | 18.60 | 45.44 | 33.13 | 49.52 | 55.11 |
| chr6 | 112481472 | 112482226 | NM_198239 | WISP3 | 10.18 | 14.31 | 10.80 | 8.75 | 8.97 | 7.70 | 10.56 |
| chr6 | 127838001 | 127839330 | NM_014702 | KIAA0408 | 57.23 | 45.95 | 57.14 | 59.45 | 52.71 | 62.56 | 65.57 |
| chr6 | 160140502 | 160141661 | NM_173516 | PNLDC1 | 18.64 | 22.33 | 18.80 | 21.10 | 27.10 | 11.83 | 10.67 |
| chr6 | 160599193 | 160600752 | NM_003058 | SLC22A2 | 17.40 | 26.17 | 15.76 | 22.01 | 13.69 | 14.66 | 12.10 |
| chr7 | 1063268 | 1064522 | NM_138445 | GPR146 | 18.19 | 27.25 | 21.97 | 15.76 | 18.52 | 12.01 | 13.65 |
| chr7 | 2684888 | 2685852 | NM_133463 | AMZ1 | 9.68 | 10.92 | 8.30 | 13.76 | 9.15 | 10.18 | 5.76 |
| chr7 | 4135803 | 4136475 | NM_001079653 | SDK1 | 8.31 | 9.05 | 5.82 | 9.95 | 6.50 | 11.32 | 7.20 |
| chr7 | 23715637 | 23716691 | NM_032944 | STK31 | 36.46 | 44.10 | 32.08 | 48.20 | 36.80 | 31.60 | 25.99 |
| chr7 | 53070155 | 53071394 | NM_182595 | POM121L12 | 12.76 | 17.02 | 10.02 | 12.28 | 12.62 | 14.16 | 10.45 |
| chr7 | 72077349 | 72078691 | NM_198853 | TRIM74 | 50.92 | 70.98 | 35.74 | 49.79 | 57.37 | 48.43 | 43.20 |
| chr7 | 72379763 | 72380692 | NM_003602 | FKBP6 | 53.21 | 58.89 | 50.39 | 53.85 | 56.06 | 44.60 | 55.48 |
| chr7 | 72486451 | 72488015 | NM_003508 | FZD9 | 13.83 | 14.57 | 10.44 | 11.94 | 12.16 | 17.88 | 16.01 |
| chr7 | 72882630 | 72883979 | NM_001305 | CLDN4 | 10.76 | 10.89 | 7.26 | 11.76 | 9.37 | 11.85 | 13.41 |
| chr7 | 74862153 | 74863417 | NM_198924 | TRIM73 | 64.84 | 79.30 | 54.71 | 64.21 | 67.97 | 57.16 | 65.69 |
| chr7 | 116854422 | 116855586 | NR_023315 | ASZ1 | 35.29 | 39.67 | 24.22 | 50.04 | 36.93 | 33.63 | 27.25 |
| chr7 | 129808524 | 129809253 | NM_001868 | CPA1 | 11.26 | 13.37 | 9.48 | 13.43 | 13.52 | 10.70 | 7.03 |
| chr7 | 129917633 | 129920382 | NM_002402 | MEST | 25.48 | 28.05 | 25.11 | 29.63 | 27.20 | 21.73 | 21.17 |
| chr7 | 142978433 | 142979168 | NM_001008747 | LOC441294 | 10.95 | 12.33 | 9.95 | 12.17 | 9.57 | 9.22 | 12.48 |
| chr7 | 149699704 | 149700939 | NM_014374 | REPIN1 | 19.81 | 7.65 | 18.54 | 25.00 | 23.02 | 27.12 | 17.53 |
| chr7 | 150376098 | 150377462 | NM_020322 | ACCN3 | 14.81 | 20.46 | 11.26 | 15.42 | 19.76 | 12.07 | 9.86 |
| chr7 | 154493038 | 154494284 | NM_024012 | HTR5A | 20.73 | 23.90 | 15.83 | 21.26 | 19.84 | 18.40 | 25.17 |
| chr8 | 8597079 | 8598233 | NM_194284 | CLDN23 | 11.15 | 9.25 | 9.96 | 13.86 | 9.49 | 13.83 | 10.50 |
| chr8 | 21825506 | 21826155 | NM_003974 | DOK2 | 13.75 | 17.08 | 14.93 | 15.84 | 13.48 | 11.21 | 9.95 |
| chr8 | 22188020 | 22189349 | NM_001135721 | PIWIL2 | 26.18 | 32.45 | 22.37 | 31.25 | 25.08 | 25.03 | 20.88 |
| chr8 | 43265944 | 43266912 | NM_001005365 | POTEA | 7.89 | 8.85 | 7.36 | 8.81 | 7.14 | 7.38 | 7.79 |
| chr8 | 86760698 | 86762541 | NR_003594 | REXO1L2P | 74.86 | 97.36 | 61.15 | 85.63 | 98.07 | 48.47 | 58.51 |
| chr8 | 88955074 | 88955655 | NM_152418 | WDR21C | 27.81 | 29.48 | 23.43 | 28.25 | 27.06 | 30.73 | 27.92 |
| chr8 | 143912441 | 143913671 | NM_002066 | GML | 19.26 | 24.67 | 22.56 | 20.03 | 16.10 | 18.46 | 13.75 |
| chr8 | 145018352 | 145018996 | NM_031308 | EPPK1 | 9.82 | 12.17 | 9.24 | 10.36 | 11.31 | 9.47 | 6.40 |
| chr9 | 106804 | 109189 | NM_207305 | FOXD4 | 9.02 | 12.94 | 8.09 | 10.01 | 6.60 | 8.38 | 8.11 |
| chr9 | 2707903 | 2708962 | NM_133497 | KCNV2 | 27.14 | 37.08 | 11.73 | 28.00 | 18.16 | 40.68 | 27.22 |
| chr9 | 5175227 | 5176457 | NM_007179 | INSL6 | 51.27 | 72.50 | 56.04 | 51.63 | 51.21 | 31.22 | 44.99 |
| chr9 | 42357193 | 42358439 | NM_001012419 | ANKRD20A3 | 7.60 | 5.98 | 8.23 | 9.35 | 7.56 | 7.79 | 6.71 |
| chr9 | 42708104 | 42709237 | NM_199244 | FOXD4L4 | 7.11 | 7.48 | 5.99 | 7.06 | 7.49 | 8.13 | 6.52 |
| chr9 | 43123727 | 43124986 | NM_001012421 | ANKRD20A2 | 7.62 | 8.32 | 8.41 | 8.30 | 5.86 | 6.92 | 7.88 |
| chr9 | 67515302 | 67516141 | NM_001012419 | ANKRD20A3 | 9.00 | 7.86 | 10.22 | 8.70 | 9.62 | 7.62 | 9.96 |
| chr9 | 68490213 | 68491448 | NM_001085476 | FOXD4L6 | 10.18 | 10.48 | 7.32 | 11.78 | 10.65 | 12.88 | 7.99 |
| chr9 | 69667338 | 69668675 | NM_199244 | FOXD4L4 | 9.35 | 8.75 | 7.28 | 12.34 | 9.31 | 10.91 | 7.53 |
| chr9 | 70106903 | 70108958 | NM_199135 | FOXD4L3 | 9.26 | 10.48 | 5.83 | 9.24 | 9.84 | 10.67 | 9.48 |
| chr9 | 70818133 | 70818792 | NM_002732 | PRKACG | 13.34 | 12.92 | 12.08 | 17.39 | 12.64 | 14.12 | 10.91 |
| chr9 | 103396630 | 103397877 | NM_147180 | PPP3R2 | 18.30 | 23.07 | 19.92 | 21.89 | 14.86 | 16.26 | 13.82 |
| chr9 | 110657158 | 110658802 | NM_006686 | ACTL7B | 47.75 | 59.69 | 42.00 | 50.58 | 52.28 | 46.30 | 35.65 |
| chr9 | 135433836 | 135434510 | NM_001080515 | FAM163B | 10.11 | 14.29 | 13.68 | 11.11 | 7.08 | 7.77 | 6.69 |
| chr9 | 135490511 | 135491455 | NM_000787 | DBH | 7.64 | 10.60 | 6.42 | 8.26 | 10.22 | 5.59 | 4.77 |
| chr9 | 137552429 | 137553278 | NM_002297 | LCN1 | 6.11 | 6.44 | 5.54 | 6.63 | 7.75 | 5.01 | 5.30 |
| chr9 | 138858099 | 138861468 | NM_001080482 | LOC389813 | 15.49 | 10.55 | 16.63 | 23.08 | 10.98 | 13.18 | 18.53 |
| chrX | 169244 | 169903 | NM_012227 | GTPBP6 | 17.18 | 23.22 | 9.47 | 16.37 | 17.65 | 21.36 | 15.03 |
| chrX | 170609 | 171148 | NM_012227 | GTPBP6 | 11.18 | 12.30 | 10.26 | 10.94 | 12.61 | 12.21 | 8.77 |
| chrX | 1673869 | 1674498 | NM_004043 | ASMT | 18.69 | 22.83 | 15.76 | 22.90 | 18.02 | 13.94 | 18.67 |
| chrX | 18148158 | 18149002 | NM_153346 | BEND2 | 54.90 | 45.78 | 46.81 | 63.36 | 58.08 | 79.46 | 35.89 |
| chrX | 18149156 | 18149705 | NM_153346 | BEND2 | 16.82 | 14.17 | 15.12 | 22.50 | 20.73 | 17.09 | 11.31 |
| chrX | 21583950 | 21587200 | NM_153270 | KLHL34 | 17.19 | 16.12 | 13.40 | 16.65 | 11.57 | 31.45 | 13.95 |
| chrX | 21783926 | 21784775 | NM_206923 | YY2 | 22.96 | 26.31 | 9.09 | 26.27 | 21.62 | 29.42 | 25.03 |
| chrX | 27735245 | 27736219 | NM_182506 | MAGEB10 | 10.81 | 16.08 | 8.36 | 9.57 | 11.64 | 9.40 | 9.83 |
| chrX | 30174411 | 30175660 | NM_177404 | MAGEB1 | 25.68 | 34.17 | 16.93 | 27.11 | 29.73 | 22.66 | 23.45 |
| chrX | 30999683 | 31000252 | NM_031894 | FTHL17 | 11.27 | 10.99 | 10.29 | 17.15 | 13.18 | 8.38 | 7.64 |
| chrX | 34058424 | 34059693 | NM_203408 | FAM47A | 68.87 | 86.96 | 30.50 | 86.50 | 35.83 | 91.33 | 82.10 |
| chrX | 36936001 | 36936845 | NM_001013736 | FAM47C | 20.67 | 23.10 | 18.45 | 19.48 | 20.62 | 18.42 | 23.98 |
| chrX | 44587800 | 44588965 | NM_022076 | DUSP21 | 18.92 | 15.84 | 15.23 | 18.97 | 23.29 | 18.38 | 21.83 |
| chrX | 48050422 | 48051291 | NM_174962 | SSX9 | 6.13 | 7.27 | 5.87 | 6.30 | 6.46 | 5.22 | 5.66 |
| chrX | 48156111 | 48156755 | NM_001034832 | SSX4B | 8.29 | 8.80 | 6.72 | 8.19 | 11.05 | 6.49 | 8.47 |
| chrX | 48341834 | 48342497 | NM_017883 | WDR13 | 18.28 | 21.49 | 7.06 | 10.95 | 13.87 | 45.48 | 10.85 |
| chrX | 51165707 | 51167656 | NM_203407 | CXorf67 | 30.06 | 26.64 | 29.35 | 29.25 | 30.28 | 29.65 | 35.19 |
| chrX | 52255448 | 52255907 | NM_001097603 | XAGE1E | 15.51 | 18.30 | 15.91 | 13.64 | 13.99 | 16.40 | 14.80 |
| chrX | 52276731 | 52277175 | NM_020411 | XAGE1D | 16.11 | 17.98 | 15.41 | 18.66 | 16.36 | 14.24 | 13.99 |
| chrX | 52528399 | 52528858 | NM_001097603 | XAGE1E | 15.58 | 18.77 | 16.59 | 16.27 | 12.57 | 14.89 | 14.38 |
| chrX | 52549671 | 52550115 | NM_020411 | XAGE1D | 17.06 | 16.87 | 17.12 | 19.40 | 16.96 | 14.91 | 17.10 |
| chrX | 52562565 | 52563009 | NM_020411 | XAGE1D | 16.04 | 17.61 | 16.98 | 16.91 | 17.95 | 14.70 | 12.09 |
| chrX | 52752781 | 52753645 | NM_003147 | SSX2 | 11.21 | 11.18 | 10.03 | 9.54 | 14.42 | 10.75 | 11.35 |
| chrX | 52796355 | 52796804 | NM_003147 | SSX2 | 8.08 | 7.33 | 6.05 | 9.61 | 10.11 | 7.78 | 7.60 |
| chrX | 52857157 | 52858012 | NM_130775 | XAGE5 | 27.36 | 36.00 | 25.93 | 30.23 | 29.31 | 16.74 | 25.93 |
| chrX | 52912981 | 52914510 | NM_133179 | XAGE3 | 17.89 | 20.88 | 16.78 | 16.36 | 18.48 | 17.19 | 17.62 |
| chrX | 55117439 | 55118384 | NM_001015038 | PAGE2B | 8.81 | 10.48 | 9.18 | 7.68 | 9.73 | 6.09 | 9.70 |
| chrX | 55131445 | 55132374 | NM_207339 | PAGE2 | 9.52 | 9.86 | 9.62 | 11.23 | 12.40 | 5.98 | 8.04 |
| chrX | 55263129 | 55263764 | NM_001013435 | PAGE5 | 10.17 | 12.20 | 7.56 | 12.50 | 12.84 | 7.98 | 7.94 |
| chrX | 69198489 | 69200038 | NM_207320 | OTUD6A | 33.00 | 33.09 | 27.30 | 34.12 | 34.16 | 41.48 | 27.87 |
| chrX | 73440474 | 73441108 | NM_203303 | ZCCHC13 | 12.81 | 13.74 | 10.46 | 11.17 | 16.43 | 15.32 | 9.71 |
| chrX | 89063104 | 89063753 | NM_138960 | TGIF2LX | 10.59 | 12.27 | 7.49 | 11.61 | 10.35 | 6.96 | 14.84 |
| chrX | 102078480 | 102079429 | NM_001031834 | RAB40AL | 8.30 | 9.49 | 5.80 | 8.41 | 8.73 | 8.19 | 9.18 |
| chrX | 103154408 | 103155677 | NM_001002916 | H2BFWT | 24.83 | 22.71 | 21.06 | 23.44 | 22.93 | 35.42 | 23.40 |
| chrX | 119094651 | 119095297 | NM_032498 | RHOXF2 | 20.02 | 23.85 | 14.66 | 20.84 | 13.48 | 29.42 | 17.88 |
| chrX | 119176907 | 119177556 | NM_001099685 | RHOXF2B | 17.65 | 22.01 | 11.11 | 23.40 | 11.90 | 25.91 | 11.54 |
| chrX | 125513403 | 125515157 | NM_178470 | WDR40B | 10.39 | 9.37 | 6.67 | 10.16 | 6.79 | 24.20 | 5.12 |
| chrX | 132179867 | 132180816 | NM_016521 | TFDP3 | 26.46 | 38.56 | 12.65 | 40.28 | 31.43 | 25.11 | 10.75 |
| chrX | 134802666 | 134803605 | NM_018666 | SAGE1 | 9.00 | 11.98 | 8.52 | 10.38 | 8.94 | 6.35 | 7.82 |
| chrX | 139923943 | 139924587 | NM_139019 | SPANXF1 | 6.42 | 7.29 | 5.56 | 6.09 | 6.06 | 6.11 | 7.38 |
| chrX | 140164130 | 140164970 | NM_022661 | SPANXC | 8.14 | 7.39 | 6.91 | 8.42 | 8.46 | 7.76 | 9.90 |
| chrX | 140500305 | 140500869 | NM_013453 | SPANXA1 | 8.44 | 10.10 | 7.36 | 6.11 | 6.70 | 9.26 | 11.09 |
| chrX | 140504824 | 140505753 | NM_013453 | SPANXA1 | 8.60 | 9.24 | 8.46 | 7.50 | 9.77 | 7.10 | 9.52 |
| chrX | 140614068 | 140614937 | NM_145665 | SPANXE | 8.98 | 7.94 | 7.84 | 10.97 | 9.14 | 8.10 | 9.86 |
| chrX | 146869951 | 146870810 | NM_152578 | FMR1NB | 8.85 | 10.34 | 6.35 | 7.08 | 7.03 | 11.36 | 10.93 |
| chrX | 152592892 | 152593731 | NM_001039582 | PNCK | 7.58 | 9.74 | 5.68 | 6.25 | 10.39 | 9.25 | 4.20 |
| chrX | 153062148 | 153063092 | NM_020061 | OPN1LW | 14.39 | 21.95 | 18.35 | 12.02 | 13.99 | 11.20 | 8.82 |
| chrX | 153176437 | 153177581 | NM_012253 | TKTL1 | 21.48 | 31.34 | 16.87 | 21.52 | 19.53 | 28.13 | 11.51 |
| chrX | 153465818 | 153466862 | NM_139250 | CTAG1A | 14.38 | 11.35 | 11.43 | 10.83 | 14.24 | 29.08 | 9.38 |
| chrX | 153500530 | 153501564 | NM_001327 | CTAG1B | 12.64 | 10.65 | 11.44 | 9.73 | 13.54 | 19.87 | 10.63 |
| chrX | 153534848 | 153535884 | NM_020994 | CTAG2 | 16.20 | 15.41 | 13.14 | 19.12 | 14.37 | 24.40 | 10.78 |
| chrX | 153765716 | 153766871 | NM_001007524 | F8A3 | 8.49 | 10.35 | 6.27 | 7.00 | 7.13 | 12.69 | 7.47 |
| chrX | 154262827 | 154263982 | NM_001007524 | F8A3 | 9.26 | 12.96 | 7.00 | 10.82 | 6.47 | 12.48 | 5.87 |
| chrX | 154342433 | 154343563 | NM_001017991 | H2AFB2 | 7.74 | 8.83 | 5.64 | 7.68 | 6.44 | 10.98 | 6.84 |
| chrX | 154763420 | 154764572 | NM_001145149 | VAMP7 | 10.52 | 10.43 | 10.18 | 15.57 | 10.27 | 8.47 | 8.18 |
| chrY | 169244 | 169903 | NM_012227 | GTPBP6 | 18.35 | 24.35 | 11.54 | 15.58 | 18.73 | 26.38 | 13.52 |
| chrY | 170609 | 171148 | NM_012227 | GTPBP6 | 11.32 | 12.22 | 8.11 | 10.13 | 13.55 | 12.21 | 11.73 |
| chrY | 1673869 | 1674498 | NM_004043 | ASMT | 20.62 | 26.62 | 16.35 | 21.79 | 19.50 | 16.29 | 23.19 |
| chrY | 14607596 | 14608245 | NM_004679 | VCY | 7.26 | 7.00 | 5.61 | 7.58 | 6.21 | 7.53 | 9.64 |
| chrY | 14676912 | 14677741 | NM_181880 | VCY1B | 16.85 | 16.64 | 13.82 | 16.24 | 16.71 | 18.58 | 19.09 |
| chrY | 57622620 | 57623772 | NM_001145149 | VAMP7 | 10.81 | 10.80 | 14.79 | 14.35 | 8.30 | 8.42 | 8.18 |

TABLE 5
Frequently methylated 5′ gene ends in brain tumors.

| Peaks | | | Average P | No. of Tumors | Downstream | | | Gene | Within | |
|---|---|---|---|---|---|---|---|---|---|---|
| chr | Start | End | value score | with Peaks | Strand | Start | End | Symbol | Strand | Start |
| chr1 | 38002438 | 38003587 | 10.10 | 12 | | | | | | |
| chr1 | 60311689 | 60312524 | 9.01 | 16 | | | | | | |
| chr1 | 63556597 | 63560345 | 8.49 | 15 | | | | | | |
| chr1 | 150078663 | 150079332 | 11.19 | 11 | | | | | + | 150077568 |
| chr1 | 153557083 | 153557827 | 8.77 | 11 | − | 153556874 | 153560562 | C1orf104 | | |
| chr1 | 179719350 | 179719904 | 10.69 | 13 | | | | | | |
| chr1 | 181421423 | 181422187 | 9.18 | 19 | | | | | | |
| chr1 | 207914587 | 207915953 | 6.93 | 9 | + | 207915292 | 207916358 | G0S2 | + | 207915292 |
| chr1 | 222870205 | 222872650 | 9.25 | 10 | | | | | + | 222870801 |
| chr1 | 234752777 | 234754328 | 9.67 | 11 | | | | | + | 234748187 |
| chr10 | 46412477 | 46413703 | 7.66 | 21 | | | | | | |
| chr10 | 50486546 | 50487915 | 9.95 | 11 | | | | | | |
| chr10 | 50488946 | 50491715 | 10.13 | 15 | + | 50488352 | 50490772 | SLC18A3 | + | 50488352 |
| chr10 | 79066119 | 79066773 | 9.27 | 12 | | | | | − | 78314641 |
| chr10 | 120344105 | 120345734 | 7.81 | 11 | | | | | − | 120342905 |
| chr10 | 124628733 | 124629607 | 7.80 | 12 | | | | | | |
| chr11 | 10428000 | 10428654 | 7.76 | 16 | | | | | | |
| chr11 | 31789500 | 31795000 | 8.20 | 10 | | | | | | |
| chr11 | 32411453 | 32412722 | 13.77 | 15 | | | | | − | 32365900 |
| chr11 | 63823533 | 63825290 | 17.92 | 13 | + | 63815368 | 63824079 | KCNK4 | | |
| chr12 | 42231009 | 42232738 | 12.35 | 17 | | | | | | |
| chr12 | 47975997 | 47977646 | 11.74 | 21 | | | | | + | 47975175 |
| chr12 | 55168271 | 55169242 | 10.27 | 19 | | | | | | |
| chr12 | 55918204 | 55919848 | 21.03 | 9 | | | | | − | 55914952 |
| chr12 | 79626169 | 79626805 | 12.87 | 20 | + | 79625576 | 79627382 | MYF6 | + | 79625576 |
| chr12 | 84197229 | 84198765 | 8.80 | 11 | | | | | | |
| chr13 | 40928925 | 40929564 | 6.50 | 17 | | | | | | |
| chr13 | 52211145 | 52212607 | 7.69 | 14 | | | | | | |
| chr13 | 102224101 | 102224965 | 10.63 | 16 | | | | | | |
| chr14 | 36122209 | 36123483 | 8.76 | 9 | | | | | | |
| chr14 | 36196539 | 36197988 | 8.90 | 13 | | | | | + | 36196532 |
| chr14 | 53491636 | 53492707 | 12.27 | 14 | | | | | − | 53486204 |
| chr14 | 56344682 | 56346630 | 8.36 | 9 | | | | | − | 56337177 |
| chr14 | 60021741 | 60022400 | 6.81 | 12 | | | | | | |
| chr14 | 60045099 | 60047942 | 9.30 | 11 | + | 60045690 | 60048278 | SIX6 | + | 60045690 |
| chr14 | 73776450 | 73777601 | 6.38 | 8 | | | | | + | 73775927 |
| chr14 | 74147083 | 74147922 | 9.41 | 19 | | | | | − | 74034638 |
| chr14 | 94304390 | 94305319 | 7.99 | 17 | − | 94304312 | 94306252 | GSC | − | 94304312 |
| chr14 | 105023673 | 105024599 | 8.21 | 12 | | | | | | |
| chr15 | 50867775 | 50868924 | 6.92 | 18 | | | | | − | 50836644 |
| chr15 | 72207930 | 72209773 | 11.51 | 13 | | | | | + | 72208767 |
| chr15 | 81112195 | 81112934 | 8.26 | 16 | | | | | − | 81009005 |
| chr15 | 82113943 | 82114802 | 6.20 | 20 | | | | | | |
| chr16 | 62621 | 63380 | 5.69 | 14 | | | | | | |
| chr16 | 19804086 | 19804635 | 8.37 | 20 | | | | | | |
| chr16 | 31014172 | 31014616 | 5.57 | 13 | | | | | | |
| chr16 | 53523342 | 53524273 | 7.02 | 16 | | | | | + | 53522611 |
| chr16 | 55229290 | 55229739 | 8.87 | 19 | | | | | | |
| chr16 | 55234406 | 55235250 | 6.28 | 12 | | | | | | |
| chr17 | 4635389 | 4636518 | 8.20 | 10 | − | 4635320 | 4636469 | VMO1 | − | 4635320 |
| chr17 | 4638694 | 4639358 | 7.56 | 13 | | | | | | |
| chr17 | 6866627 | 6868275 | 12.02 | 20 | | | | | + | 6867092 |
| chr17 | 6886114 | 6887363 | 7.45 | 14 | − | 6885672 | 6887966 | SLC16A11 | − | 6885672 |
| chr17 | 36937436 | 36938490 | 8.11 | 8 | | | | | | |
| chr17 | 41329931 | 41330876 | 7.71 | 22 | | | | | + | 41328944 |
| chr17 | 42251317 | 42252051 | 10.35 | 11 | | | | | | |
| chr17 | 44564528 | 44565791 | 10.73 | 10 | | | | | | |
| chr17 | 55853774 | 55854910 | 6.09 | 16 | | | | | | |
| chr17 | 69719958 | 69720487 | 9.44 | 11 | | | | | − | 69717735 |
| chr17 | 69781509 | 69782358 | 7.08 | 11 | | | | | | |
| chr17 | 77479685 | 77480419 | 7.24 | 13 | | | | | + | 77478995 |
| chr18 | 5186243 | 5187292 | 11.77 | 12 | | | | | − | 5133671 |
| chr18 | 26875494 | 26877443 | 12.24 | 16 | | | | | − | 26824049 |
| chr18 | 53254565 | 53259819 | 10.42 | 18 | | | | | + | 53253914 |
| chr18 | 55091928 | 55092557 | 11.94 | 15 | | | | | | |
| chr18 | 72664418 | 72665346 | 7.60 | 10 | | | | | | |
| chr19 | 1812475 | 1814434 | 8.50 | 13 | | | | | − | 1803398 |
| chr19 | 9469724 | 9470804 | 10.30 | 11 | | | | | | |
| chr19 | 10258672 | 10259721 | 8.63 | 13 | + | 10258649 | 10260198 | ICAM4 | + | 10258649 |
| chr19 | 10558593 | 10559142 | 6.31 | 12 | | | | | | |
| chr19 | 18389860 | 18391505 | 11.85 | 15 | | | | | | |
| chr19 | 40222452 | 40224104 | 9.89 | 21 | + | 40213373 | 40223193 | SCN1B | | |
| chr19 | 49455836 | 49456370 | 7.46 | 11 | | | | | | |
| chr19 | 53891560 | 53892099 | 7.52 | 10 | | | | | + | 53891039 |
| chr19 | 59157320 | 59157774 | 9.54 | 19 | | | | | | |
| chr2 | 10100088 | 10101856 | 10.60 | 16 | | | | | | |
| chr2 | 72226653 | 72228187 | 10.04 | 12 | | | | | − | 72209874 |
| chr2 | 74582914 | 74584780 | 8.71 | 8 | − | 74585677 | 74588329 | PCGF1 | + | 74583251 |
| chr2 | 111591681 | 111594348 | 13.30 | 21 | + | 111206620 | 111592270 | ACOXL | | |
| chr2 | 115636321 | 115637260 | 13.24 | 12 | | | | | + | 115636201 |
| chr2 | 119322147 | 119323502 | 10.24 | 13 | | | | | | |
| chr2 | 119630604 | 119631968 | 10.75 | 17 | − | 119630288 | 119632941 | C1QL2 | − | 119630288 |
| chr2 | 119905436 | 119906957 | 8.58 | 13 | | | | | + | 119905915 |
| chr2 | 155263352 | 155264303 | 12.69 | 17 | | | | | | |
| chr2 | 158161857 | 158162509 | 7.18 | 12 | | | | | − | 158091524 |
| chr2 | 160626827 | 160628085 | 6.25 | 14 | | | | | | |
| chr2 | 172673432 | 172675478 | 7.47 | 13 | | | | | − | 172672411 |
| chr2 | 176653956 | 176656685 | 9.31 | 11 | − | 176653080 | 176656936 | EVX2 | − | 176653080 |
| chr2 | 176671999 | 176672948 | 7.88 | 16 | + | 176672775 | 176673734 | HOXD12 | + | 176672775 |
| chr2 | 176690151 | 176690900 | 10.31 | 17 | | | | | + | 176689737 |
| chr2 | 176737592 | 176738247 | 9.42 | 16 | | | | | + | 176737050 |
| chr2 | 200041833 | 200043073 | 13.73 | 23 | | | | | + | 200041065 |
| chr2 | 222869621 | 222872193 | 7.97 | 14 | | | | | + | 222871109 |
| chr2 | 232103046 | 232104293 | 8.25 | 15 | | | | | | |
| chr2 | 232279406 | 232280745 | 13.71 | 9 | | | | | | |
| chr2 | 233442461 | 233443405 | 8.93 | 17 | | | | | | |
| chr20 | 22505521 | 22506890 | 10.23 | 14 | | | | | − | 22489193 |
| chr20 | 25076779 | 25077618 | 6.77 | 13 | | | | | | |
| chr20 | 41251999 | 41252763 | 10.05 | 12 | | | | | | |
| chr20 | 41976531 | 41978482 | 10.08 | 20 | | | | | + | 41976905 |
| chr20 | 43469538 | 43470502 | 10.23 | 12 | | | | | + | 43468650 |
| chr20 | 43531029 | 43532764 | 7.57 | 11 | | | | | | |
| chr21 | 21291294 | 21291968 | 10.35 | 10 | | | | | | |
| chr21 | 34962773 | 34964708 | 7.05 | 10 | | | | | + | 34963557 |
| chr22 | 28039290 | 28041531 | 13.41 | 21 | − | 28038921 | 28041748 | RASL10A | − | 28038921 |
| chr22 | 36807805 | 36808832 | 5.62 | 12 | | | | | − | 36804089 |
| chr3 | 2115949 | 2117089 | 11.08 | 19 | | | | | | |
| chr3 | 32254374 | 32255424 | 7.95 | 11 | | | | | | |
| chr3 | 50357760 | 50358604 | 8.66 | 9 | | | | | | |
| chr3 | 138020672 | 138021936 | 18.36 | 10 | | | | | + | 138020550 |
| chr3 | 155629021 | 155629670 | 10.35 | 10 | | | | | − | 155538154 |
| chr3 | 159303324 | 159304476 | 14.83 | 12 | | | | | − | 159296493 |
| chr3 | 161649899 | 161651150 | 6.32 | 16 | | | | | | |
| chr3 | 173648073 | 173649732 | 9.01 | 18 | | | | | | |
| chr3 | 188870393 | 188870937 | 13.49 | 19 | | | | | | |
| chr4 | 5945893 | 5946532 | 7.96 | 15 | | | | | | |
| chr4 | 52612173 | 52613018 | 7.73 | 17 | | | | | | |
| chr4 | 54661877 | 54662826 | 14.11 | 17 | + | 54661004 | 54662879 | GSX2 | + | 54661004 |
| chr4 | 85636686 | 85637940 | 7.21 | 15 | | | | | − | 85633459 |
| chr4 | 122211474 | 122212323 | 18.69 | 9 | | | | | − | 122176231 |
| chr4 | 174688523 | 174689572 | 9.32 | 15 | | | | | + | 174688183 |
| chr5 | 2805770 | 2806724 | 9.19 | 18 | | | | | + | 2805261 |
| chr5 | 32747979 | 32750229 | 12.42 | 16 | | | | | + | 32747421 |
| chr5 | 37872516 | 37875378 | 15.39 | 13 | | | | | − | 37851509 |
| chr5 | 63292326 | 63293075 | 10.85 | 20 | | | | | | |
| chr5 | 76151227 | 76151876 | 8.32 | 17 | | | | | + | 76150588 |
| chr5 | 76968098 | 76969322 | 7.73 | 9 | | | | | − | 76960292 |
| chr5 | 140286488 | 140287347 | 15.62 | 17 | | | | | + | 140242037 |
| chr5 | 140742560 | 140743119 | 8.07 | 13 | | | | | + | 140733834 |
| chr5 | 140747458 | 140748016 | 8.51 | 17 | | | | | + | 140742650 |
| chr5 | 140767256 | 140768329 | 11.36 | 19 | | | | | + | 140762703 |
| chr5 | 145698688 | 145700318 | 10.46 | 12 | + | 145698779 | 145700276 | POU4F3 | + | 145698779 |
| chr5 | 157030866 | 157031611 | 9.76 | 19 | | | | | | |
| chr5 | 158690162 | 158691536 | 7.47 | 12 | | | | | | |
| chr6 | 10994972 | 10995711 | 7.30 | 13 | | | | | | |
| chr6 | 26125798 | 26126758 | 9.25 | 13 | | | | | | |
| chr6 | 27948718 | 27949880 | 10.00 | 11 | − | 27948904 | 27949268 | HIST1H4L | − | 27948904 |
| chr6 | 30050074 | 30050923 | 5.05 | 13 | | | | | | |
| chr6 | 30818753 | 30820599 | 10.08 | 16 | − | 30818954 | 30820306 | IER3 | − | 30818954 |
| chr6 | 43720000 | 43721360 | 8.21 | 12 | | | | | | |
| chr6 | 78228956 | 78230415 | 10.96 | 17 | | | | | | |
| chr6 | 83130068 | 83131208 | 8.72 | 14 | | | | | | |
| chr6 | 112147783 | 112148610 | 9.95 | 13 | | | | | − | 112089177 |
| chr6 | 117192435 | 117193290 | 8.00 | 14 | | | | | − | 117180053 |
| chr6 | 137857127 | 137857980 | 13.22 | 14 | | | | | | |
| chr6 | 150326335 | 150328184 | 32.01 | 19 | | | | | + | 150326835 |
| chr7 | 526958 | 527907 | 9.01 | 15 | | | | | | |
| chr7 | 5077779 | 5078727 | 6.16 | 15 | + | 5078263 | 5079380 | LOC389458 | + | 5078263 |
| chr7 | 27100570 | 27102300 | 8.47 | 12 | | | | | − | 27099138 |
| chr7 | 27156749 | 27161918 | 7.67 | 13 | − | 27159862 | 27162821 | HOXA7 | − | 27159862 |
| chr7 | 27185469 | 27186510 | 12.70 | 12 | − | 27187300 | 27191360 | HOXA11 | | |
| chr7 | 27190648 | 27192605 | 14.02 | 16 | | | | | + | 27191551 |
| chr7 | 27248657 | 27253218 | 8.83 | 9 | + | 27248688 | 27252717 | EVX1 | + | 27248688 |
| chr7 | 51352205 | 51352779 | 7.08 | 20 | | | | | | |
| chr7 | 116906858 | 116907422 | 10.33 | 12 | | | | | | |
| chr7 | 120755525 | 120756474 | 10.97 | 17 | | | | | + | 120752656 |
| chr7 | 129912506 | 129914030 | 8.21 | 18 | − | 129914133 | 129918249 | MESTIT1 | | |
| chr7 | 131983416 | 131984045 | 7.46 | 10 | | | | | | |
| chr7 | 156125196 | 156126661 | 8.12 | 9 | | | | | | |
| chr8 | 10626015 | 10628284 | 7.94 | 10 | | | | | | |
| chr8 | 23618422 | 23620876 | 8.71 | 14 | | | | | − | 23615909 |
| chr8 | 24826911 | 24828465 | 8.26 | 19 | | | | | + | 24827178 |
| chr8 | 24868882 | 24870630 | 9.36 | 13 | | | | | − | 24864387 |
| chr8 | 26777575 | 26780521 | 15.03 | 22 | | | | | − | 26683138 |
| chr8 | 57522214 | 57523567 | 8.06 | 16 | | | | | | |
| chr8 | 65873955 | 65874718 | 9.27 | 15 | | | | | | |
| chr8 | 71144440 | 71147178 | 6.16 | 11 | | | | | − | 71126576 |
| chr8 | 98359200 | 98360130 | 6.68 | 12 | | | | | | |
| chr8 | 102573379 | 102574213 | 9.31 | 19 | | | | | | |
| chr8 | 110725080 | 110726354 | 9.55 | 13 | | | | | − | 110655580 |
| chr9 | 34369432 | 34371281 | 7.87 | 20 | − | 34369016 | 34387849 | C9orf24 | − | 34369016 |
| chr9 | 85760867 | 85761816 | 9.92 | 8 | | | | | | |
| chr9 | 90339041 | 90340305 | 7.18 | 17 | | | | | | |
| chrX | 111211507 | 111212571 | 11.31 | 13 | | | | | − | 110904197 |

| Peaks | | | | Gene | Upstream | | | Gene |
|---|---|---|---|---|---|---|---|---|
| chr | Start | End | End | Symbol | Strand | Start | End | Symbol |
| chr1 | 38002438 | 38003587 | | | − | 37998531 | 38003411 | EPHA10 |
| chr1 | 60311689 | 60312524 | | | − | 60228653 | 60312014 | C1orf87 |
| chr1 | 63556597 | 63560345 | | | + | 63561317 | 63563385 | FOXD3 |
| chr1 | 150078663 | 150079332 | 150083264 | LOC100132111 | − | 150076962 | 150079657 | LOC100191040 |
| chr1 | 153557083 | 153557827 | | | + | 153557263 | 153567533 | RUSC1 |
| chr1 | 179719350 | 179719904 | | | + | 179719338 | 180037338 | CACNA1E |
| chr1 | 181421423 | 181422187 | | | + | 181421796 | 181480885 | LAMC2 |
| chr1 | 207914587 | 207915953 | 207916358 | G0S2 | + | 207915292 | 207916358 | G0S2 |
| chr1 | 222870205 | 222872650 | 222994872 | CNIH3 | + | 222870801 | 222994872 | CNIH3 |
| chr1 | 234752777 | 234754328 | 234779619 | LGALS8 | + | 234753699 | 234779619 | LGALS8 |
| chr10 | 46412477 | 46413703 | | | + | 46413551 | 46420574 | GPRIN2 |
| chr10 | 50486546 | 50487915 | | | + | 50488352 | 50490772 | SLC18A3 |
| chr10 | 50488946 | 50491715 | 50490772 | SLC18A3 | + | 50492088 | 50543156 | CHAT |
| chr10 | 79066119 | 79066773 | 79067583 | KCNMA1 | − | 78314641 | 79067583 | KCNMA1 |
| chr10 | 120344105 | 120345734 | 120345150 | PRLHR | − | 120342905 | 120345150 | PRLHR |
| chr10 | 124628733 | 124629607 | | | + | 124629138 | 124648220 | LOC399815 |
| chr11 | 10428000 | 10428654 | | | + | 10428799 | 10485702 | AMPD3 |
| chr11 | 31789500 | 31795000 | | | − | 31768059 | 31789361 | PAX6 |
| chr11 | 32411453 | 32412722 | 32413663 | WT1 | − | 32365900 | 32413663 | WT1 |
| chr11 | 63823533 | 63825290 | | | + | 63824438 | 63828815 | C11orf20 |
| chr12 | 42231009 | 42232738 | | | − | 42034278 | 42231991 | ADAMTS20 |
| chr12 | 47975997 | 47977646 | 47978748 | PRPH | + | 47975175 | 47978748 | PRPH |
| chr12 | 55168271 | 55169242 | | | − | 55151002 | 55168448 | GLS2 |
| chr12 | 55918204 | 55919848 | 55920742 | NDUFA4L2 | − | 55914952 | 55920742 | NDUFA4L2 |
| chr12 | 79626169 | 79626805 | 79627382 | MYF6 | + | 79625576 | 79627382 | MYF6 |
| chr12 | 84197229 | 84198765 | | | + | 84198166 | 84219692 | ALX1 |
| chr13 | 40928925 | 40929564 | | | + | 40929541 | 40943013 | C13orf15 |
| chr13 | 52211145 | 52212607 | | | − | 52175399 | 52211948 | LECT1 |
| chr13 | 102224101 | 102224965 | | | − | 102216465 | 102224150 | C13orf27 |
| chr14 | 36122209 | 36123483 | | | − | 36118966 | 36121537 | NKX2-8 |
| chr14 | 36196539 | 36197988 | 36216762 | PAX9 | + | 36196532 | 36216762 | PAX9 |
| chr14 | 53491636 | 53492707 | 53493304 | BMP4 | − | 53486204 | 53493304 | BMP4 |
| chr14 | 56344682 | 56346630 | 56346937 | OTX2 | − | 56337177 | 56346937 | OTX2 |
| chr14 | 60021741 | 60022400 | | | − | 59972426 | 60022517 | C14orf39 |
| chr14 | 60045099 | 60047942 | 60048278 | SIX6 | + | 60045690 | 60048278 | SIX6 |
| chr14 | 73776450 | 73777601 | 73799194 | VSX2 | + | 73775927 | 73799194 | VSX2 |
| chr14 | 74147083 | 74147922 | 74148787 | LTBP2 | − | 74034638 | 74148787 | LTBP2 |
| chr14 | 94304390 | 94305319 | 94306252 | GSC | − | 94304312 | 94306252 | GSC |
| chr14 | 105023673 | 105024599 | | | + | 105024301 | 105026169 | CRIP1 |
| chr15 | 50867775 | 50868924 | 50869501 | ONECUT1 | − | 50836644 | 50869501 | ONECUT1 |
| chr15 | 72207930 | 72209773 | 72216196 | ISLR2 | + | 72209795 | 72216196 | ISLR2 |
| chr15 | 81112195 | 81112934 | 81113783 | CPEB1 | − | 81009005 | 81113783 | CPEB1 |
| chr15 | 82113943 | 82114802 | | | + | 82113841 | 82499597 | ADAMTSL3 |
| chr16 | 62621 | 63380 | | | − | 48057 | 62629 | RHBDF1 |
| chr16 | 19804086 | 19804635 | | | − | 19777793 | 19803652 | GPRC5B |
| chr16 | 31014172 | 31014616 | | | − | 31009675 | 31013777 | VKORC1 |
| chr16 | 53523342 | 53524273 | 53525896 | IRX5 | + | 53522611 | 53525896 | IRX5 |
| chr16 | 55229290 | 55229739 | | | + | 55230078 | 55231500 | MT1A |
| chr16 | 55234406 | 55235250 | | | + | 55235216 | 55236353 | MT1DP |
| chr17 | 4635389 | 4636518 | 4636469 | VMO1 | − | 4635320 | 4636469 | VMO1 |
| chr17 | 4638694 | 4639358 | | | + | 4638993 | 4640425 | GLTPD2 |
| chr17 | 6866627 | 6868275 | 6873685 | BCL6B | + | 6867092 | 6873685 | BCL6B |
| chr17 | 6886114 | 6887363 | 6887966 | SLC16A11 | − | 6885672 | 6887966 | SLC16A11 |
| chr17 | 36937436 | 36938490 | | | − | 36933394 | 36938167 | KRT19 |
| chr17 | 41329931 | 41330876 | 41331959 | LOC100130148 | + | 41328944 | 41331959 | LOC100130148 |
| chr17 | 42251317 | 42252051 | | | − | 42196856 | 42251081 | WNT3 |
| chr17 | 44564528 | 44565791 | | | + | 44565327 | 44602350 | B4GALNT2 |
| chr17 | 55853774 | 55854910 | | | + | 55854646 | 55863568 | C17orf64 |
| chr17 | 69719958 | 69720487 | 69721055 | MGC16275 | + | 69721290 | 69769752 | TTYH2 |
| chr17 | 69781509 | 69782358 | | | + | 69781991 | 69822617 | DNAI2 |
| chr17 | 77479685 | 77480419 | 77481919 | LOC92659 | + | 77478995 | 77481919 | LOC92659 |
| chr18 | 5186243 | 5187292 | 5187255 | LOC642597 | − | 5133671 | 5187255 | LOC642597 |
| chr18 | 26875494 | 26877443 | 26876779 | DSC3 | − | 26824049 | 26876779 | DSC3 |
| chr18 | 53254565 | 53259819 | 53309528 | ONECUT2 | + | 53253914 | 53309528 | ONECUT2 |
| chr18 | 55091928 | 55092557 | | | − | 55085246 | 55091605 | RAX |
| chr18 | 72664418 | 72665346 | | | + | 72665103 | 72811670 | ZNF236 |
| chr19 | 1812475 | 1814434 | 1814564 | KLF16 | − | 1803398 | 1814564 | KLF16 |
| chr19 | 9469724 | 9470804 | | | − | 9438030 | 9470279 | ZNF560 |
| chr19 | 10258672 | 10259721 | 10260198 | ICAM4 | + | 10258649 | 10260198 | ICAM4 |
| chr19 | 10558593 | 10559142 | | | − | 10544347 | 10558991 | AP1M2 |
| chr19 | 18389860 | 18391505 | | | + | 18391135 | 18406459 | SSBP4 |
| chr19 | 40222452 | 40224104 | | | + | 40223249 | 40249315 | HPN |
| chr19 | 49455836 | 49456370 | | | + | 49455915 | 49471308 | ZNF233 |
| chr19 | 53891560 | 53892099 | 53901003 | FUT2 | + | 53891039 | 53901003 | FUT2 |
| chr19 | 59157320 | 59157774 | | | + | 59158105 | 59177951 | CACNG8 |
| chr2 | 10100088 | 10101856 | | | + | 10101132 | 10112414 | KLF11 |
| chr2 | 72226653 | 72228187 | 72228471 | CYP26B1 | − | 72209874 | 72228471 | CYP26B1 |
| chr2 | 74582914 | 74584780 | 74585700 | LOC151534 | + | 74583251 | 74585700 | LOC151534 |
| chr2 | 111591681 | 111594348 | | | + | 111594961 | 111642493 | BCL2L11 |
| chr2 | 115636321 | 115637260 | 116318406 | DPP10 | + | 115636201 | 116318406 | DPP10 |
| chr2 | 119322147 | 119323502 | | | − | 119316216 | 119322229 | EN1 |
| chr2 | 119630604 | 119631968 | 119632941 | C1QL2 | − | 119630288 | 119632941 | C1QL2 |
| chr2 | 119905436 | 119906957 | 119912566 | TMEM37 | + | 119905915 | 119912566 | TMEM37 |
| chr2 | 155263352 | 155264303 | | | + | 155263338 | 155421260 | KCNJ3 |
| chr2 | 158161857 | 158162509 | 158193645 | ACVR1C | − | 158091524 | 158162318 | ACVR1C |
| chr2 | 160626827 | 160628085 | | | − | 160510571 | 160627367 | PLA2R1 |
| chr2 | 172673432 | 172675478 | 172675724 | DLX2 | − | 172672411 | 172675724 | DLX2 |
| chr2 | 176653956 | 176656685 | 176656936 | EVX2 | − | 176653080 | 176656936 | EVX2 |
| chr2 | 176671999 | 176672948 | 176673734 | HOXD12 | + | 176672775 | 176673734 | HOXD12 |
| chr2 | 176690151 | 176690900 | 176692916 | HOXD10 | + | 176689737 | 176692916 | HOXD10 |
| chr2 | 176737592 | 176738247 | 176746072 | HOXD3 | + | 176737050 | 176746072 | HOXD3 |
| chr2 | 200041833 | 200043073 | 200045724 | FLJ32063 | + | 200041065 | 200045724 | FLJ32063 |
| chr2 | 222869621 | 222872193 | 222878180 | CCDC140 | + | 222871109 | 222878180 | CCDC140 |
| chr2 | 232103046 | 232104293 | | | − | 232096114 | 232103426 | NMUR1 |
| chr2 | 232279406 | 232280745 | | | + | 232281478 | 232286494 | PTMA |
| chr2 | 233442461 | 233443405 | | | + | 233443237 | 233449351 | UNQ830 |
| chr20 | 22505521 | 22506890 | 22507280 | C20orf56 | − | 22489193 | 22507280 | C20orf56 |
| chr20 | 25076779 | 25077618 | | | − | 25069433 | 25077426 | LOC284798 |
| chr20 | 41251999 | 41252763 | | | − | 40134805 | 41251971 | PTPRT |
| chr20 | 41976531 | 41978482 | 42131668 | TOX2 | + | 41978195 | 42131668 | TOX2 |
| chr20 | 43469538 | 43470502 | 43472661 | DBNDD2 | + | 43470328 | 43472661 | DBNDD2 |
| chr20 | 43531029 | 43532764 | | | + | 43531807 | 43543586 | WFDC2 |
| chr21 | 21291294 | 21291968 | | | + | 21292503 | 21833085 | NCAM2 |
| chr21 | 34962773 | 34964708 | 35012389 | CLIC6 | + | 34963557 | 35012389 | CLIC6 |
| chr22 | 28039290 | 28041531 | 28041748 | RASL10A | − | 28038921 | 28041748 | RASL10A |
| chr22 | 36807805 | 36808832 | 36809116 | SLC16A8 | − | 36804089 | 36809116 | SLC16A8 |
| chr3 | 2115949 | 2117089 | | | + | 2117246 | 3074645 | CNTN4 |
| chr3 | 32254374 | 32255424 | | | + | 32255174 | 32386817 | CMTM8 |
| chr3 | 50357760 | 50358604 | | | − | 50353540 | 50358160 | ZMYND10 |
| chr3 | 138020672 | 138021936 | 138057424 | TMEM22 | + | 138020550 | 138057424 | TMEM22 |
| chr3 | 155629021 | 155629670 | 155630198 | GPR149 | − | 155538154 | 155630198 | GPR149 |
| chr3 | 159303324 | 159304476 | 159306630 | SHOX2 | − | 159296493 | 159303520 | SHOX2 |
| chr3 | 161649899 | 161651150 | | | − | 161635984 | 161650320 | TRIM59 |
| chr3 | 173648073 | 173649732 | | | − | 173648027 | 173648897 | GHSR |
| chr3 | 188870393 | 188870937 | | | − | 188869387 | 188870895 | SST |
| chr4 | 5945893 | 5946532 | | | − | 5873391 | 5945686 | CRMP1 |
| chr4 | 52612173 | 52613018 | | | + | 52612349 | 52658215 | SPATA18 |
| chr4 | 54661877 | 54662826 | 54662879 | GSX2 | + | 54661004 | 54662879 | GSX2 |
| chr4 | 85636686 | 85637940 | 85638411 | NKX6-1 | − | 85633459 | 85638411 | NKX6-1 |
| chr4 | 122211474 | 122212323 | 122213123 | C4orf31 | − | 122176231 | 122213123 | C4orf31 |
| chr4 | 174688523 | 174689572 | 174699556 | NBLA00301 | + | 174688183 | 174699556 | NBLA00301 |
| chr5 | 2805770 | 2806724 | 2808511 | C5orf38 | + | 2805261 | 2808511 | C5orf38 |
| chr5 | 32747979 | 32750229 | 32823011 | NPR3 | + | 32747421 | 32823011 | NPR3 |
| chr5 | 37872516 | 37875378 | 37875539 | GDNF | − | 37851509 | 37875539 | GDNF |
| chr5 | 63292326 | 63293075 | | | − | 63292033 | 63293302 | HTR1A |
| chr5 | 76151227 | 76151876 | 76166895 | F2RL1 | + | 76150588 | 76166895 | F2RL1 |
| chr5 | 76968098 | 76969322 | 76970278 | OTP | − | 76960292 | 76970278 | OTP |
| chr5 | 140286488 | 140287347 | 140372113 | PCDHA13 | + | 140286485 | 140372113 | PCDHAC1 |
| chr5 | 140742560 | 140743119 | 140872730 | PCDHGA6 | + | 140742650 | 140872730 | PCDHGA7 |
| chr5 | 140747458 | 140748016 | 140872730 | PCDHGA7 | + | 140747635 | 140872730 | PCDHGB4 |
| chr5 | 140767256 | 140768329 | 140872730 | PCDHGA9 | + | 140767953 | 140872730 | PCDHGB6 |
| chr5 | 145698688 | 145700318 | 145700276 | POU4F3 | + | 145698779 | 145700276 | POU4F3 |
| chr5 | 157030866 | 157031611 | | | + | 157031138 | 157039738 | C5orf52 |
| chr5 | 158690162 | 158691536 | | | − | 158674368 | 158690059 | IL12B |
| chr6 | 10994972 | 10995711 | | | + | 10995049 | 11082527 | SYCP2L |
| chr6 | 26125798 | 26126758 | | | − | 26125238 | 26126019 | HIST1H1A |
| chr6 | 27948718 | 27949880 | 27949268 | HIST1H4L | − | 27948904 | 27949268 | HIST1H4L |
| chr6 | 30050074 | 30050923 | | | + | 30050870 | 30054156 | HCG9 |
| chr6 | 30818753 | 30820599 | 30820306 | IER3 | − | 30818954 | 30820306 | IER3 |
| chr6 | 43720000 | 43721360 | | | + | 43720787 | 43746722 | RSPH9 |
| chr6 | 78228956 | 78230415 | | | − | 78228666 | 78229839 | HTR1B |
| chr6 | 83130068 | 83131208 | | | + | 83130257 | 83133335 | TPBG |
| chr6 | 112147783 | 112148610 | 112301320 | FYN | − | 112089177 | 112147958 | FYN |
| chr6 | 117192435 | 117193290 | 117193579 | FAM162B | − | 117180053 | 117193579 | FAM162B |
| chr6 | 137857127 | 137857980 | | | − | 137855028 | 137857224 | OLIG3 |
| chr6 | 150326335 | 150328184 | 150336539 | ULBP1 | + | 150326835 | 150336539 | ULBP1 |
| chr7 | 526958 | 527907 | | | − | 503424 | 526007 | PDGFA |
| chr7 | 5077779 | 5078727 | 5079380 | LOC389458 | + | 5078263 | 5079380 | LOC389458 |
| chr7 | 27100570 | 27102300 | 27102150 | HOXA1 | − | 27099138 | 27102150 | HOXA1 |
| chr7 | 27156749 | 27161918 | 27162821 | HOXA7 | − | 27159862 | 27162821 | HOXA7 |
| chr7 | 27185469 | 27186510 | | | − | 27176734 | 27186401 | HOXA10 |
| chr7 | 27190648 | 27192605 | 27195437 | HOXA11AS | + | 27191551 | 27195437 | HOXA11AS |
| chr7 | 27248657 | 27253218 | 27252717 | EVX1 | + | 27248688 | 27252717 | EVX1 |
| chr7 | 51352205 | 51352779 | | | − | 51051402 | 51352009 | COBL |
| chr7 | 116906858 | 116907422 | | | + | 116907252 | 117095954 | CFTR |
| chr7 | 120755525 | 120756474 | 120768394 | WNT16 | + | 120756325 | 120768394 | WNT16 |
| chr7 | 129912506 | 129914030 | | | + | 129913281 | 129933367 | MEST |
| chr7 | 131983416 | 131984045 | | | − | 131820060 | 131983987 | PLXNA4 |
| chr7 | 156125196 | 156126661 | | | + | 156126161 | 156162581 | RNF32 |
| chr8 | 10626015 | 10628284 | | | − | 10618687 | 10625432 | SOX7 |
| chr8 | 23618422 | 23620876 | 23619867 | NKX2-6 | − | 23615909 | 23619867 | NKX2-6 |
| chr8 | 24826911 | 24828465 | 24832511 | NEFM | + | 24828359 | 24832511 | NEFM |
| chr8 | 24868882 | 24870630 | 24870048 | NEFL | − | 24864387 | 24870048 | NEFL |
| chr8 | 26777575 | 26780521 | 26778839 | ADRA1A | − | 26683138 | 26778839 | ADRA1A |
| chr8 | 57522214 | 57523567 | | | − | 57516068 | 57521836 | PENK |
| chr8 | 65873955 | 65874718 | | | − | 65671082 | 65873902 | CYP7B1 |
| chr8 | 71144440 | 71147178 | 71146116 | PRDM14 | − | 71126576 | 71146116 | PRDM14 |
| chr8 | 98359200 | 98360130 | | | − | 98354889 | 98359352 | TSPYL5 |
| chr8 | 102573379 | 102574213 | | | + | 102573843 | 102751128 | GRHL2 |
| chr8 | 110725080 | 110726354 | 110773196 | GOLSYN | − | 110655580 | 110726998 | GOLSYN |
| chr9 | 34369432 | 34371281 | 34387849 | C9orf24 | − | 34369016 | 34371598 | C9orf24 |
| chr9 | 85760867 | 85761816 | | | − | 85743046 | 85761483 | C9orf64 |
| chr9 | 90339041 | 90340305 | | | + | 90339835 | 90380523 | NXNL2 |
| chrX | 111211507 | 111212571 | 111212660 | TRPC5 | + | 111212908 | 111587129 | ZCCHC16 |

TABLE 6
Genes with frequent hypomethylation at the 5′ end

| | | | | | | | Annotated to refseq | | |
|---|---|---|---|---|---|---|---|---|---|
| Peaks | | | Average P value | Average P value | No. of Tumors with | | | | Gene |
| chr | Start | End | score in Normal | score in Tumor | hypomethylation | Strand | Start | End | Symbol |
| chr1 | 55043525 | 55044789 | 9.92 | 0.90 | 29 | + | 55044323 | 55080525 | C1orf177 |
| chr1 | 245760561 | 245761190 | 11.23 | 0.17 | 30 | − | 245754603 | 245760729 | LOC148824 |
| chr10 | 105207770 | 105209419 | 8.15 | 0.76 | 29 | − | 105203133 | 105208638 | CALHM1 |
| chr10 | 134590561 | 134591211 | 14.89 | 0.13 | 30 | − | 134471886 | 134590989 | C10orf92 |
| chr10 | 135086692 | 135087332 | 6.87 | 0.00 | 30 | − | 135084159 | 135088111 | SPRN |
| chr10 | 135228897 | 135229736 | 10.54 | 0.41 | 30 | − | 135218337 | 135229128 | SYCE1 |
| chr11 | 50213739 | 50214494 | 20.02 | 0.70 | 30 | − | 50195574 | 50214199 | LOC441601 |
| chr11 | 94439813 | 94440372 | 9.32 | 0.44 | 29 | + | 94439703 | 94444036 | SFRS2B |
| chr12 | 50004030 | 50004674 | 6.53 | 0.00 | 28 | − | 49961088 | 50004205 | BIN2 |
| chr13 | 35686378 | 35687238 | 8.02 | 0.00 | 30 | − | 35640346 | 35686752 | SOHLH2 |
| chr14 | 69107587 | 69109936 | 15.81 | 0.35 | 30 | − | 69106284 | 69107671 | C14orf162 |
| chr15 | 22471456 | 22472405 | 12.17 | 0.27 | 30 | + | 22471633 | 22479686 | C15orf2 |
| chr16 | 66571411 | 66572180 | 13.68 | 1.17 | 27 | − | 66567066 | 66571953 | DPEP3 |
| chr16 | 74126384 | 74127343 | 8.12 | 0.97 | 26 | − | 74119928 | 74126569 | CHST5 |
| chr17 | 23658080 | 23658835 | 6.71 | 0.65 | 26 | − | 23627734 | 23658144 | FLJ40504 |
| chr17 | 25927568 | 25928103 | 6.43 | 0.63 | 26 | + | 25927608 | 25988611 | LRRC37B2 |
| chr18 | 27594502 | 27595268 | 19.93 | 0.67 | 30 | − | 27593656 | 27594841 | MCART2 |
| chr18 | 59294602 | 59295373 | 7.41 | 0.45 | 27 | + | 59295123 | 59323298 | SERPINB5 |
| chr19 | 16861645 | 16862379 | 14.99 | 0.95 | 30 | + | 16860825 | 16863830 | F2RL3 |
| chr19 | 37902256 | 37902885 | 10.24 | 0.12 | 30 | + | 37902518 | 37973554 | TDRD12 |
| chr19 | 51818645 | 51819269 | 8.01 | 0.00 | 30 | − | 51815564 | 51820194 | PTGIR |
| chr19 | 53656820 | 53657569 | 21.31 | 0.64 | 30 | + | 53656317 | 53661179 | KCNJ14 |
| chr2 | 106450965 | 106451409 | 14.15 | 0.78 | 30 | − | 106387567 | 106451233 | RGPD3 |
| chr2 | 107809732 | 107810061 | 7.36 | 0.61 | 29 | + | 107809819 | 107875431 | RGPD4 |
| chr2 | 108702582 | 108703121 | 13.69 | 0.59 | 30 | + | 108702368 | 108768699 | RANBP2 |
| chr2 | 131918756 | 131919690 | 16.19 | 0.14 | 30 | − | 131916205 | 131918937 | LOC401010 |
| chr2 | 238371764 | 238372800 | 28.42 | 0.80 | 30 | + | 238372126 | 238416189 | RBM44 |
| chr20 | 4176544 | 4177393 | 10.90 | 0.63 | 28 | − | 4149277 | 4177659 | ADRA1D |
| chr20 | 29689051 | 29689700 | 5.41 | 0.43 | 27 | + | 29689351 | 29696461 | COX4I2 |
| chr20 | 61963314 | 61964663 | 12.67 | 0.30 | 30 | + | 61963009 | 61964785 | C20orf135 |
| chr22 | 35231985 | 35232930 | 14.68 | 0.80 | 30 | − | 35213178 | 35233094 | FOXRED2 |
| chr22 | 48869825 | 48871184 | 9.64 | 0.19 | 30 | + | 48870620 | 48942243 | MOV10L1 |
| chr3 | 48673342 | 48674613 | 9.92 | 0.36 | 29 | − | 48648899 | 48675352 | CELSR3 |
| chr5 | 177546540 | 177547089 | 11.17 | 0.15 | 30 | − | 177544116 | 177547039 | GMCL1L |
| chr6 | 66554331 | 66554795 | 5.01 | 0.00 | 30 | + | 66554492 | 66556096 | MCART3P |
| chr7 | 2684888 | 2685852 | 9.68 | 0.13 | 30 | + | 2685688 | 2721595 | AMZ1 |
| chr8 | 8597079 | 8598233 | 11.15 | 0.96 | 27 | + | 8597075 | 8599027 | CLDN23 |
| chr9 | 41944310 | 41945459 | 13.06 | 1.11 | 26 | − | 41942398 | 41945076 | MGC21881 |
| chr9 | 44185465 | 44186109 | 15.45 | 1.12 | 29 | + | 44185511 | 44246438 | KGFLP1 |
| chr9 | 66292609 | 66293870 | 12.00 | 1.11 | 27 | + | 66293050 | 66295728 | MGC21881 |
| chrX | 48050422 | 48051291 | 6.13 | 0.44 | 27 | − | 48039828 | 48050558 | SSX9 |
| chrX | 48156111 | 48156755 | 8.29 | 0.68 | 29 | − | 48146471 | 48156288 | SSX4B |
| chrX | 52668418 | 52669072 | 5.11 | 0.26 | 25 | + | 52668709 | 52679723 | SSX8 |
| chrX | 52796355 | 52796804 | 8.08 | 0.48 | 29 | + | 52797032 | 52807342 | SSX2 |
| chrX | 55263129 | 55263764 | 10.17 | 0.23 | 30 | + | 55263516 | 55267266 | PAGE5 |
| chrX | 84075635 | 84076305 | 10.44 | 1.13 | 28 | + | 84075812 | 84076551 | UBE2DNL |

DNA SEQUENCE (from UC Santa Cruz Genome
Browser, Human March 2006 (hg18) assembly)
SEQ ID NO. 1:
Chromosome 7, starting from 152252636 to
152253475
CACACCTGCAAGCTGCGTGCAACCGAAAGGCTGGCGACCTGTAGCTTG
TCAGATTCAGATATGGTGCAGGTGAGGGCCTGGCAAACATTTAGTTTA
ATTTCTGAGACCGTTAGAGACAAGGTTAAATTCCAGGTGAACGCGGTT
CCAGAGTGGCTGGGATTCCCACTCTCAGATGGGAACCCTTCAGACAAG
AGTAAAACAGGAGGGACTTCTTCGCCGCCCGCCCTGCGGTGTGTGCCA
CGGAGGCCGCTGGGTGGGGGCGTCCAAATGTGGAAGCGTTCCTCCAGC
ATGCGTGGATATCCTGAGGACTCACGGACACCGATTCCCTCTTCCGGG
CCCAGACGTCCTAATTCCCGTCCCCCTTGAGGTGAGATCCCGTTTTCC
CCCAGCCAAAGGGACTCCGGAGCCGCATTCACCTGACTTGGCTGCCAG
CGCCCGCCTTCCTCGAGTTCTCAGGGCAAGGGGCTGTGGTGCATTTTC
TGGGGATGGTTTGTGGATGTGGAAGAGGGAAATCCTTTCAGGGATTTT
AGTCATTTGGCAATTGGTGATTTTCATATGACGTGCTGGTTTTAGGGG
CCAACTCTGATAAGCTGGGACGCCCTCCCCTTGATTCTAGAACAGAAG
GCATTTTGGGCTAAGGACGCTCACAACCCGGTTTCCTGTCCCGCGTGG
TTCATGGGGCTTGCCCTGGGCCTGGGAGGCCGGTGGCGGGAGCCTCCC
CTACAGTGAGGTGGGTGGGGGGCGCCACACGGCGTCTCAGGGAGAGGG
GAGAGGCGGCTTCTGTTCCGGGAAGAGGGAACGGTGGAGGCCGCCGTT
CTCTTGGGCGCGGCCTCTGCTGGG
SEQ ID NO. 2:
Chromosome 5, starting from 158456493 to
158457132
CCAAGGCTCCCGGGCCTGCTCCCGAGCCGAGCCGGGGACGAGGGGCGA
CAGCGCTGCGCCCACTTACTTTTTCCTTCTCCACGAACCCCACAAACG
CTGTCCTCTCGATCTCCACGGGCTGGCCCTGTCTGTCGTAGAGGGCCA
GGACGAAGTGGAAGAAGTTGGATTTCCGCAGATTGGAAGGCGGCTGCT
TCTCAAAGTGAGCCCGGGCCAGACCCACCCCGCTGCGGCCAAAGACGC
AGAGTTAGATGGCTAAACCGGACGCCGACCCGCGCCCCTTGTCACCCT
GTACACACACTCGAACCCTCAAACACCCACCTCCTCTTCCCCCAAAAC
ATCCAGTGGGCGCTCTTCACGCCCCTTCAGGCGACATAGACCCAGCTG
ACAACGGGACACACATGTCCCCTCATGTCCTCGTCCTCTCCGCCAAGG
ACGGGTGCGTCCTCAGAACTCGCTGAAGAGGTGTGAAAGTTTTGTTTG
GGTTTTGCGCGCGAGATGAAACTCTATAAGCATTTACTCCTACCTGAC
ACGCACTGCGTGGGGAGGAAGAAAAGTGGCGGTGGATGAAGTGAACCC
ACCTTCGCGGAGTAGAACCCACAGTTATATTCCGGCACCCCTCGAATC
TCACGTCTCAGCTAGC
SEQ ID NO. 3:
Chromosome 12, starting from 47771091 to
47771619
CCTAGGGTGGCAACAGTACTACTGCAGACTCAGTTTCCCGGAGGGAGC
CCCCTTTGGGCGGATTTTACCACCCTCCTCCTACTGTACCAGCTTTGA
CCGACACGTGGACGTGGTTGCGGGACTCGTAGTAGACCCAGTCGAAGC
CGGCTTCCACTGCGAGGCGCGCCAGCAACCCATACTTGTTGCGGTCGC
GGTCAGACGTAGTGATGTCCAAAGCACGGCCTTCGTAGTGGAGTGAAT
CCTGAGCGTGGTGGCCGTCCTCGTCCCAGCCCTCAGTCACTCGTAGGC
GCACTCCGGGCCACATGTTCATCACGGCAATGGCCAAAGCGTTCACCC
GCTCCTTACAACGCTGGCGGGGAATAAAGGAGTCAGTCTCCCCACCAC
CACCCTTGGGGCAAAAGGGACCTGGATAGGAGGGTTGATTCTTTGTTA
TTCCGGCCCTACTCTTACCCCTCCCAGCTTTTGAGTGTCCTGGAGAAA
TGAGAATCTGAGTCGATGGTAGTCACCAATCTGCACTCTGTTCCCAGG
A
SEQ ID NO. 4:
Chromosome 14, starting from 22360909 to
22361365
CTCAGGTGACCGGTCTGGGTGCGGCGTGGAGATGGCGAGAGCTAAGTG
CTATGGCCGCGAAAGGGTGAAGGGCAGGGGAGGAAAAGGCCGAGGGGA
GGCGAACGCTCAGGTTCACACATATGCAAGTGGGCTCTACAGCGGACT
TCGAAGCATACACTCAACTCCCCACCATGCGCCGGCCCGCTGCTCTAC
CCCTGAAAAGCTCTCCCCTGGCCCGCGATCCTTGCTGGCCTACCCCTT
GGTCGTTCCCATCCCCCTCTCCGCCCCCGCCCAACCTGCTGGCCTCAC
TCACCTTTGTCCCTTTCTCCCATCCCCACCCCCCCACCACCCTCGCTT
TCTCCGGGCCCCTCCCTGTCGTTTGTCTCCCTTTTATTTCCCTCCTTC
CTTTGGGCCCCCAGTCTGCGGCCCTAGGACAACGCCTTCCCAAGCTCG
GACCCAGCCACCGTCCGCCTTCATT
SEQ ID NO. 5:
Chromosome 2, starting from 200041833 to
200043073
CCCTGATCAGTTTGTATATGAAACACTTTTTTTCCTGCCTTAAGGTTG
GAATCTGGTAAGTTTCAGACAAGTGGTTGCAGGGGGAGATGACCCAGG
GGACCGTGCGGGACAGGGCCGGTGAACCCGATTCCGCGAGGCGGGCGT
CTCGAAGGGACTGGTCTGCGTGCCTCACCGGGTTCCCCAGCCTGGCTC
CGAGGAAAGGCCGCGCGGAGGCGAGAAAGGGCCCACATCTCGAGTCTC
CAGTCTCTCCTTGAAAAGAGCACCCCCTAATCCCGGAGGAAGGGAGGG
TGAAGGAGGAGGGGAGAGCCCACTACAGGCAAGGCAGGAAGGTTAATC
ATTTTGTTAACCGGAAAGGGTGCTGCCCCGCCTGGCCGTCGTCACCGA
GCGTGTCAGTCACCCGCTCTCGATTTCGCCGCAGAATTACCTGCTCAA
AGAGCCACCCCAAGAAGCCCTCAGCTCCGAGGTCTTCCCGCCCGTCGT
CTGCGGGAAGTAAACGGCGTCCTCCCTCCAAAGAAGTGGATGCCCCTT
CGACTCTCCTCCGCCCCCCGGCGGTTCCTAAATCTAGGAAAGCCAGCC
CCGGCATCGCTCACTACTCCTGCACCTCAGCTCTGGGATACCCGCTCA
GTACAGACAGACCATCAACCAAAAAAGAAAAGAAGAAAAGAAAAGTGG
TGGTGGGGGTGGGGGTGGGCAGGGAGCGTAAAGTAGCGAGATCACTTA
TTTGACTCTTCTGTTTTCTTAGGAACCTGATCCTGGCCCCCGGCAGAG
CATAAGGGCCCGAGGCCAGGGACCTAGAGGCCGGACCAAGTTCGCGCG
AGGTGTTCACAGCCTAGGCGCCCCGGGCTTGGCTTCGTACTCTGCAAC
AAGCCAAGCTCCCTTGCCACGAGTATATCAGCAACTTCAGAAATAAAC
ACCAGCGAAACCCCTTCCCACATTAGCTTGTTGCTGTGCTACTGACAC
TGGGCTTAGTCGATGTTCACACCTATTACATTTTTCCGGAAAGTGGGA
GGAGCAGGGGGTAGAGAAAGCTGTGTGTATTGAGGGAAGGGGAGATGT
GTGCAACACCTTCCAGAAAAATGGGGAGGTAGTTTAGAAAGTTTTTCT
CTGTAACAGCACAGCTTCTGAGAAGGCAGAGAGAAGAAAGCAGACAGG
AAACGAATCCCTTCCTCCTCTCCCCTTTACCCCCTTCCTTCACTCCCT
AGACCAAGCTGGTCACCCGGCTGGGAATCAAGATTGTGTGG
SEQ ID NO. 6:
Chromosome 4, starting from 41443929 to
41444657
AAAATGCCAAGAGCGTGCGCTCTAAGCCTCTCTCGAACGCACAGCCAC
ACCAAATCCAGTATTTCTGATCGGCCATGGGGCCCTAGGTCCTTCTCA
CTCGAGGCTCCAGGACTTCGAATTTCACCAGCCGCCCCTCACCCCACA
CCTCCCCGGACCAGTGCGGCGGAGCGGGGTCGGTTTCCAGGCGCGCGT
ACCTGGACTCGCGCCTCTGTGAGGTCGATCTTCAGGGCCAGCTCCTCC
CGAGTGTAGATGTCGGGGTAGTGAGTCTCCGCGAAGACCCTTTCCAGC
TCTTTGAGCTGGGCACTGGTGAAAGTGGTGCGGATGCGCCGCTGCTTG
CGCTTCTCGTTGAGGCCGCCGTGGTCCGTGAAGAGTTTGTAAGGAACT
AGAGTATGACAGAGGAGACAGAAAGTGAGCAAATCAGCCGGCAGCTCG
CCGGCCGTGGAGCTAGAATGTGAGGACTCCAAGGTCAGGTCATACGGC
AGCCGCTACCTACACCCGCGCGAGAGAGTTGCCGAGAGAAGCTGCGGG
AAGAAACCGAGGAAATTTGTTATCTGCGGAAACCTGGGCTCAAACTTC
GGGCTTGGCGGAGTCCTTTCTGGCACAAGCGCCTTTGGGTGGATTGAA
ACAGCGCGATGTGACGGACTTGAGATAGTGCTTTCAGCAGGAAAAAAC
CAGAGAGGAAAAAAATCCAAAAACATCAGTCGGAATATCAGGGAGCCA
GGAAAGAAG
SEQ ID NO. 7:
Chromosome 17, starting from 41329931 to
41330876
ATAAATTTCACCCGTGTCCAAGTTCACCGCGCCCCCAAAACCGAGTCT
GGGGCGGCAGGGGGAACTCCTGGCCAACGAATCCATGCCTCGCCCTCC
TGTGATGAACCTGGTACGCACGGTTTTCTGGTTAATTCTATCGCTGAA
AACTGGTGCGGGGGGCGCACTTCTGAGACGGAAGAGCATCTAGGAGCT
GAATCCTCCACGCGGGTCGCCCAGGTTGATCTGAATTTCTGGGGAATG
GCTTGGCTGCCCGCCCGGGACCAGGCCGACCCTCCTTGACGGTGGCGT
AGAGGGCTGGAGCCTGGGTACTGCGAGGCTCCTCGCATGGCTGGGCCC
GCCGCGAGGGGTTGCAGAGCGGCTCAGGGATCGATTCAAGCATCGTCT
CTCCTCCCTCGCCCCCAGACAGAGCTGGGCGCGGGGTTCCCCTTCCAG
ATGGAGCGAGGGTCTCGGGGTGGCCCCGGAAAAGGGGAGCCCGCGGCC
ACGGCTACGTATTGCCATCTCGCGAGCAGAGATGTCACCTCCTGCCTT
TGGAGGAAAGGGAGCCCGGTGGGGATGAGCGCATTTAGCCCAATGCTG
GGAACAAAGCGCACTCCGCGCTTCTGCGATTTCGCTCCATTTTGAAAT
GTGTTGGCGCTTTGGTGGGGCCGCTGCGGTGGGCAAGGCCGGGGGCGC
TGTTAATGGAGGAACCTCAGGGGGACGGTCCTTCGTAGGAAACTCTAT
CCTGGCTCTGCGCGCGCTTTAAGGAAATGGCTTCCCTCCAGGACCTCG
AGGGATGCAGCTTTTGCGCGGATGACGGTGGGGTGCTGAACCAGCCGG
TGCGCCTCTGGAAATGTCTGGGCACGGATCCTGGGGCCATCGACGACT
CCTCCCCATTCCCAGCAGGCGGGAGCTCTTACATTCCGAGCGAGTGAC
CCCTCTCACCCTCTGGCGCTCACACACCTGTAAC
SEQ ID NO. 8:
Chromosome 19, starting from 56897971 to
56898600
GTCAAGATGCCACCTGGCCGATGGACCCCGGGAAGCTGCATGGATCTC
AAATGGGCCAAGGGCCCCGGGCTTGGAAGGGCAGGTGGCGCCTAGACC
CAGCGTTCCCGCTCCACAGGTCTCCAGGAGTTCGCCCGGCGTTTCCTC
TGCAGCGGGTGCTACTCTAGGGTCTGCGACCTCCCGCTGGACTGCCCA
GGTGAGGGGGCGGGGCCTCGGGGTGCAGGAGGCCAACCTGAGAAAAGG
GACCAGAGCACCGAGGGGCATAAGCTGGTGGCGAGGCCAGACGTGGTC
GGGGCCCAGCGAGTGAACCCTGCTCCGTCTTCAGTTCAGGATGTGACA
GTGACTCGGGGCGACCAGGCTATGTTTTCTTGCATCGTAAACTTCCAG
CTGCCAAAGGAGGAGATCACCTATTCCTGGAAGTTCGCAGGAGGAGGT
GTGAGTCGGGGCGGGGCCGGCGCGAAGAGTTTAGACGGGCGAGTTAGC
CTTCACTAGAGGTTGGGGAGTCAGCTTGCGGGGACGGGACTCCGGGAA
TCTCCGTCTAGACACAAGCATCAGCAAGAGGAGGGTCGCGGGATTTAG
GGGAGAGCTCGAAGATCAGAATGCAGGACCAAGGACCTGGGGCGATGG
CTCACG
SEQ ID NO. 9:
Chromosome 20, starting from 44073717 to
44074646
CTCCGACGGCTACCGCTGGTGCGCCACCACCGCCAACTACGACCGGGA
CAAGCTCTTCGGCTTCTGCCCGACCCGAGGTACCTCCACCCTGTCTAC
CAGGTTCAGCCCCGCCCTCTCATCATGTATTGGCCCCCAAAACGCGGC
TCTTCCCTCCCATCAGTTTGTCTTTCCACTCTCATTGGTCCTCAGGAC
GACCGTGACTCCGCCCACCTACACCACATTTCCACCACTATCCCTGAC
TTCCAATGGCCCCGCCCCAGCCACTAAGGTTCGGCCTTTTCTGCCCAG
CTGGCCGCCTCTTCCTTGGTCTGGTGTCCCAGGCACCGCCCACGGGTC
TAGCCTCTTCTCAGGAGTGCTCTACAGCGCCCCCTAGGCCACCAAGAT
TGTTTAGCTCCCTGTCGGGTCGGCCCCTGACTCCTTATTGGACTCATC
CATCTGGCTCATCCAAGGCCTTGGGTCTCTCCAGCTGACTCGACGGTG
ATGGGGGGCAACTCGGCGGGGGAGCTGTGCGTCTTCCCCTTCACTTTC
CTGGGTAAGGAGTACTCGACCTGTACCAGCGAGGGCCGCGGAGATGGG
CGCCTCTGGTGCGCTACCACCTCGAACTTTGACAGCGACAAGAAGTGG
GGCTTCTGCCCGGACCAAGGTAGGCGTGGTCCCGCGGCTCCGGGGCTG
GGGTTCCCGGCAGTGGTGGTGGTGGGGTGGCCAGGGCTGGGGGCTCGG
CCCGGCGCTCACGTCTCAGGCTCCCTCTCCCTCCAGGATACAGTTTGT
TCCTCGTGGCGGCGCATGAGTTCGGCCACGCGCTGGGCTTAGATCATT
CCTCAGTGCCGGAGGCGCTCATGTACCCTATGTACCGCTTCACTGAGG
GGCCCCCCTTGCATAAGGACGACGTGAATGGCATCCGGCACCTCTATG
GTGAGGCAGGGGCAGGGA
SEQ ID NO. 10:
Chromosome 8, starting from 26777575 to
26780521
CGATGCCCAGCGTTTTGGCCGCTTTCTTCTCCCGGGAGAACTTGAGGA
GCCTCACTGAGAAGTGCGTCTTGGTCTTGGCGCTGGCCATCCCGCTGC
CTCCTGCCGGGGCGTTTTTCCGATGGATGCGGAGCGTCACTTGCTCCG
AGTCCGACTTGTCGGTCTTGAGGCCAGACTTGAGGCCCCGGCTCTCCC
TCTTGGCCACCACGTAGACGCGGCAGTACATGACCAGGATGATGGCCA
GAGGCAGGTAGAAGGAGCCCAGCGCTGAGAAGAGCACGTAGCCCGGCT
CCTCGTTGATCTGGCAGATGGTCTCGTCCTCGGGGGCCGGCTGCCTCC
AGCCGAACAGGGGTCCAATGGATATGACCAGGGAGAGTGCCCAGACGC
AGAGCAGAGCCATGAGACCCCTCCTCTGGGTGACGATGGTTGGGTAGC
GCAGCGGGTAGCTCACGCCGATGTAGCGGTCGATGGAGATGATGCAGA
GGCCCATGATGGACGCGGTGCAGCACAGCACATCCACTGCCGCCCAGA
TGTTGCAGAAGACCCTGCCGAAGGCCCAGTAGCCTAGGACCTCGAAGA
TGGCGGAGAAGGGCAGCACCGTGGAGGTGAGCAGGAGGTCGGCCACCG
CCAGGTTGACGATGTAGTAGTGCGTGACTGAGTGCAGGTGTCGGTGAC
AGGCTACGGAGAGGATCACTAGGATGTTACCCAGCACCCCGAAAAGAA
TGAGGCCCCCCAAGATCACCCCGAGCAGAATGGCCTTGGAAATGTTCA
CCGGTGCCGGCGGTTGGGTGCAGTTGGAGCTGTCGGAAGCATTTCCCG
AGAGAAACACCATGGTCCCAGCCGGGGCCGGGCGAGGTCCGGCTGTCC
AGGGCCACCTCCCGGGCTGGCGCGGAGGCGGGAGCGCGGGAGCCGGGA
ATCAAAAGGTCTCGGCTGGAGGGAGCCCTGCCAGGTGGGTTTGGCTGG
GGGTGAGAGCGCGCGCGCGGGTGGGAAACAACCCTGGCCAGCCCTGGG
AACCCTCAGAAGGCCACATGAAGGGGCAGGGCATTAAAGACATGGCCA
GGGGCGCGCGGGGCTGCCGGGGACCCTCTCCACCTGCCGGGCTGGCCT
AGCCCGGGACCCGGACTCCCTCCCTACCCGAAGCTGGGTGCGAAGATC
CAGGAGACTCCTTGCAACATGCAATTCCAGAATTACGAGAATCTGCTT
TTCCGCGCTGTCTTATTCGTCCTGGCTGGGGGAAGATTCAGCATTCTG
CACATGATTCGGAATTCAAAACTCCAGCGCCAGTCTCTCCCTCAAACC
AAAAGATCAGCCGTCGACGCTCAAAGGCAGGGACCGATGGTTGGGTAG
CGCAGCGCTACAGGTTGGGCGCTGCTCCTGGCCCGCAGTTACCTACAT
TTTGAGCTGCCCCACCGAAGGCTCCTGCTCTCTCCAGCTTCTAGGAGC
ACAGGTCAGGGGACGTAGGTGTGGAATATGTGCTGAGACCCAGGAGGC
TGCGGGGCATCGTTTGACCGCGTCCACCTGAAAGAGCGCAAAGAGAAA
GGCGGCTTTGAGCTAGGCGCCCCAGGGAAAGAGGCTGTGCTGAGCTTG
ACGGGTTGGGGGACACCAGTTGGGAGCCGGGTTGGTTCTGCGGTCCAG
AAGCTGCTTCGCCCGGCAGCGGTGGAGGCGACTTCGGAGCTCATCTCG
CGCCCCCACCACTGGGAACCTGCCTAGCGCACTCTACTGAGTCACCTC
TGCCCGAGTTCAGGATCCGAAGCGAAAAACAAACAAAAAAACAAATCC
CCAAAACCCCAGGCTCCAGCGCTCGAAGTCTGGATTTCGAGCGCAGGT
ACCACGAAATTAAAATCCTCGAAGCCCCGGAGGGGCGACTGCGGCTGG
CGAGTGTGTCGCACGGATCCTAGCCGGGGAATTCTGGAGGATGTACTC
GGTTTCCGTTAGAACAGGTTGCCTCGCAGTCACCTGGAGGGGGCGGCC
TGGGAGAGGGAACAGCTGCGACCCAGGGACCTCAGGGCCAGGACCACG
AGCACGCTCACTCTCACGCCGGTGGAATTCGCACTCGGGTGTGCAGAG
CGCACCGGTCTGTCCACCAGCCAAGCTGGCTTGAGAGCCAGGTCCCGA
GCGAAGCCGGGAGGGACCCGAAGACAGAAAGCGACCCAGGTCTGTCCA
CGACGCCTTTCCAAGCCTCACTGTTGGCCACCCGAGGGGCTCCAGCTG
CCAGCCGTAGCGGGACTGGCGGGGGACGGGGTGGGGAGGACGTCCCTC
TGCAGAGGGAGCTTTGCAAGTGACAGTCACTGCGGATTTTGCAGGCGT
AAACATTAAGCCGGCATGCCAGCGGGCAGGGGCCGCCTTCGATTTTCT
GGGCTGGGGGCCACCATCTGCGTAAGAAACCTGGGTTTCCACAACTGC
GAACCTACCGCAGGGACTCGGCAGGACAGCGCGGCGTGAGGAAGTGCC
CACCTTGCCTTCTGGGGTTGGGCCCTGAGGTGGGACCTCGGGCAAAGA
CTCTTGTTAAAATCGCAAGTTGGAGACCTCACAGGTGGTATTAAAAAC
GTGCCACTGCAGAAACCCTTTTCCTCCTACCTCGAGGGTGATAAACAC
AGGGTTAAAAAGACACTTGTTCAGTAGAAGGCAACTTCGCAGAAGATG
TAAGGGAATCGGGGGTGGGGTAGAGGGGCCGGTATAAAACCTGGTGGA
AAAAGCGGGAGGCGCTGGGAAAAGTGGGGGTTCCGTCTCACCAGACGG
CGGGGGAGGTCTGCCCTCACCCACTCGGCCCTGCGGGACGCCGGCCCC
GGCGCACTCACCTGAAGCGCCGCTGCTGAGCCACCAGCTCGCGCGCGG
GGGATGTGGACCCGGCTTCGGTCCCGGGAGCTGCCTGCCTGCTCTTCT
CTGGAGGCGGAGAGGGGACCGTGTCTCCGAGGCACCAAATCCTTGTCC
TTTTGCACCCGCTGACTCA

1. A composition of matter comprising a DNA hypermethylation brain cancer marker on acellular DNA of a human subject selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, and combinations thereof.

2. A method of diagnosing a brain tumor in a human subject comprising:
providing a sample containing acellular DNA from the human subject;
determining whether one or more DNA hypermethylation markers are methylated on the acellular DNA; and
detecting the methylation of the DNA hypermethylation markers is indicative of brain cancer.

3. The method of claim 2, wherein the DNA hypermethylation markers are selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, and combinations thereof.

4. The method of claim 2, wherein the DNA hypermethylation marker comprises CpG clusters associated with homeobox genes.

5. The method of claim 4, wherein the homeobox genes are selected from the group consisting of HOX genes clusters HOXA, HOXB, HOXC, HOXD, the homeobox genes DLX1, BARHL2, PITX2, developmental transcription factor gene simple-minded one (SIM1), NKX2-8 PAX9 and FOXA1.

6. The method of claim 2, wherein the brain cancer diagnosed is a brain tumor.

7. The method of claim 6, wherein the brain tumor is a grade I tumor.

8. The method of claim 2, wherein the sample is selected from the group consisting of a blood sample, serum sample and plasma sample.

9. The method of claim 3, wherein the brain cancer diagnosed is a brain tumor.

10. The method of claim 9, wherein the brain tumor is a grade I tumor.

11. The method of claim 3, wherein the sample is selected from the group consisting of a blood sample, serum sample and plasma sample.

12. The method of claim 4, wherein the brain cancer diagnosed is a brain tumor.

13. The method of claim 12, wherein the brain tumor is a grade I tumor.

14. The method of claim 4, wherein the sample is selected from the group consisting of a blood sample, serum sample and plasma sample.

15. The method of claim 5, wherein the brain cancer diagnosed is a brain tumor.

16. The method of claim 15, wherein the brain tumor is a grade I tumor.

17. The method of claim 5, wherein the sample is selected from the group consisting of a blood sample, serum sample and plasma sample.

18. The method of claim 6, wherein the sample is selected from the group consisting of a blood sample, serum sample and plasma sample.

19. The method of claim 9, wherein the sample is selected from the group consisting of a blood sample, serum sample and plasma sample.

20. The method of claim 12, wherein the sample is selected from the group consisting of a blood sample, serum sample and plasma sample.
